(19) United States
(12) Patent Application Publication
(10) Pub. No.: US 2007/0003955 A1
(43) Pub. Date: Jan. 4, 2007

(54) Normalization of samples for amplification reactions

(76) Inventors: Natalia Novoradovskaya, San Diego, CA (US); Alexey Novoradovsky, San Diego, CA (US); Joseph A. Sorge, Del Mar, CA (US); Lee Scott Basehore, Lakeside, CA (US); Jeffery Carl Braman, Carlsbad, CA (US)

Correspondence Address: LATIMER IP LAW, LLP, 13873 PARK CENTER ROAD, SUITE 122, HERNDON, VA, 20171, US

(21) Appl. No.: 11/453,262

(22) Filed: Jun. 15, 2006

Related U.S. Application Data
(63) Continuation-in-part of application No. 11/152,775, filed on Jun. 15, 2005.

(51) Int. Cl. C12Q 1/68; C12P 19/34

(52) U.S. Cl. 435/6; 435/91.2

(57) ABSTRACT

The present invention provides oligonucleotide primers, compositions, methods, and kits for determining the amount of gDNA or the number of cells from which a cell lysate originated. The invention relies on detection and amplification of unique genomic sequences within the genome of an organism of interest to determine the amount of genomic nucleic acid in a sample. The invention can be used to normalize samples from particular cells, cell types, or organism, and provide an accurate basis for comparison of expression of genes across the samples.

### CROSS-REFERENCE TO RELATED APPLICATIONS

The present application is a continuation-in-part application of U.S. patent application Ser. No. 11/152,775, filed on 15 Jun. 2005, the entire disclosure of which is hereby incorporated herein in its entirety by reference.

### BACKGROUND OF THE INVENTION

1. Field of the Invention

The present invention relates to the field of molecular biology. More specifically, the present invention relates to quantitating and normalizing the amount of starting materials provided for amplification of nucleic acids, such as by polymerase chain reaction (PCR) techniques.

2. Description of Related Art

Amplification of nucleic acids is now a routine procedure for analysis of target sequences, including those containing genomic or sub-genomic sequences and those of expressed genes. The polymerase chain reaction (PCR) has made such analyses possible. Indeed, numerous PCR techniques are now available for analysis of different nucleic acids, including stable or stably expressed nucleic acids such as genomic DNA or rRNA, and unstable or transient nucleic acids such as mRNA and short regulatory RNAs (e.g., miRNA, siRNA). Analysis of expressed nucleic acids has become an important tool in studying development of organisms, tissues, and diseases and disorders, and PCR techniques to do so, such as reverse transcriptase PCR (RT-PCR), have been developed to permit such analysis.

RT-PCR is the method of choice for analyzing mRNA levels in samples. In RT-PCR, mRNA is first copied to cDNA by a reverse transcriptase. A PCR reaction is then set up, which includes a buffer containing at least one thermostable polymerase (such as Taq polymerase), specific primers for the mRNA of interest, deoxynucleotides, and any salts or other components that are desired. Optionally, the template mRNA is degraded during first strand cDNA synthesis or during the PCR reaction. After first strand synthesis, the cDNA is denatured by heating to separate the two strands. The sample is cooled to about 50° C. to 60° C., during which the specific primers specifically hybridize to complementary sequences on the target cDNA. Amplification of the target cDNA is accomplished by extension of the primers by the thermostable polymerase at about 72° C. After extension of the primers, the resulting mixture is heated at greater than about 90° C. to denature double-stranded products, and the process of primer annealing and extension is repeated. Cycles of annealing, extending, and denaturation are performed at least until a detectable amount of product is produced, and typically between 25 and 35 cycles are performed. Although it requires amplification of target mRNA, which has the potential to introduce errors in sequences and causes difficulties in quantitating original amounts of target mRNA, RT-PCR is more sensitive than other techniques for detecting mRNA, such as Northern blotting and RNase protection.

When performing an analysis of expressed nucleic acids, it is typically important to understand the level or amount of expression of the nucleic acid compared to other nucleic acids. This desire reflects the recognition that many cellular functions are regulated by gene expression levels or changes in gene expression levels of certain genes or sets of genes. Accordingly, quantitation of transcription levels is often important in understanding a biological state, whether it be a basal level of cell maintenance or a disease state. Quantitating nucleic acid levels is also important when detecting contamination of samples, including samples destined as food sources for animals or humans. For example, in testing for the presence of infectious or otherwise dangerous microbes in animal or human food, it is often important to know how many microbes are present in the sample. Knowing the amount of contamination permits the food producer and government regulatory agencies to make determinations as to the safety of the food before it enters the supply chain. However, a basic RT-PCR procedure does not permit one to draw accurate conclusions about the original expression level of the mRNA target or its relative abundance compared to mRNA of other samples.

Real-time, quantitative reverse transcriptase polymerase chain reaction (QRT-PCR) has been shown to be useful in determining, in real-time, the amount of mRNA of interest in a sample. It was developed to address the shortcomings of RT-PCR with regard to quantitating mRNA levels. QRT-PCR is the most sensitive method currently available for detecting and quantitating mRNA, and has become the method of choice for validating results of other techniques that assay gene expression, such as microarrays.

QRT-PCR was originally made possible by the discovery that certain polymerases, including Taq polymerase, have, in addition to their polymerase activity, a 5′-3′ exonuclease activity. This activity was first used to design probes that alter their fluorescence when in solution or digested into their component nucleotides (i.e., TaqMan® (Applied Biosystems, Foster City, Calif.) probes). Since that time, other probes and primers have been designed to take advantage of changes in fluorescence as a function of binding to target nucleic acids. Changes in fluorescence of these probes and primers can be used, in real time, to follow amplification of target mRNA, and to quantitate mRNA levels. For example, TaqMan®, Scorpions, and Molecular Beacons show different fluorescence levels when bound to nucleic acids or free in solution. TaqMan® probes, Scorpions, and Molecular Beacons comprise both a fluorescent moiety and quencher on the same molecule. TaqMan® probes rely on degradation by a polymerase to generate a detectable signal, while Scorpions and Molecular Beacons rely on opening of a hairpin structure to provide a detectable signal. More specifically, for TaqMan® probes, when the probe is intact, the quencher quenches the signal produced by the fluorescent label. However, upon binding of the probe to the target sequence and subsequent digestion of the probe by the 5′-3′ exonuclease activity of a polymerase, such as Taq polymerase, the fluorescent moiety is released from the quencher moiety, and a detectable signal, which is proportional to the amount of target nucleic acid being produced, is produced and can be monitored. Like TaqMan® probes, Scorpion probes contain both a fluorescent moiety and quenching moiety on a single probe. However, unlike TaqMan® probes, Scorpions are not degraded during the amplification reaction. Rather, they are designed as primers for amplification reactions. Scorpion primers are designed to form hairpin structures in solution, which causes the fluorescent moiety and the quenching moiety to be in close proximity. Binding of the primers to target nucleic acids unfolds the hairpin structure and moves the quenching moiety a sufficient distance away from the fluorescent moiety that detectable fluorescence is emitted. In the Molecular Beacons system, a hairpin probe is provided that binds to the target nucleic acid. Upon binding, the hairpin unfolds, permitting production of a detectable signal. Unlike TaqMan® probes, the probe is not degraded upon amplification of the target sequence. In addition, unlike Scorpions, the Molecular Beacon probe is not incorporated into the final product. The SYBR® Green (Molecular Probes, Eugene, Oreg.) system is a simple and cost-effective way to detect and quantitate PCR products in real time. The SYBR® Green dye binds, in a sequence non-specific manner, to double-stranded nucleic acids. It thus can be used for detection and quantitation of double-stranded products produced from single-stranded templates (e.g., mRNA). Other detectable probes and primers, such as Sunrise™ primers, amplifluor probes, and DNAzymes, have been used for quantitative detection of amplification products.

Multiplexing of PCR reactions is now common. Multiplexing allows the practitioner to assay two or more different targets in a single reaction through the use of multiple probes or primers, each specific for its own target and each comprising a fluorescent moiety that emits at a unique wavelength (as compared to the other probes). Multiplexing is possible with TaqMan® probes, Molecular Beacons, and Scorpions. Due to its non-specific binding nature, SYBR® Green is not amenable to multiplexing.

Quantitating starting amounts of target nucleic acids, such as mRNA in QRT-PCR reactions, is critical to drawing conclusions about the role of a particular gene in a disease or disorder, in development of a tissue, or in an infectious or toxigenic process. However, current methods of quantitating PCR targets lack the required accuracy and repeatability to draw valid conclusions across multiple samples. The shortcomings of these techniques are due mainly to slight differences in amounts of starting materials or in choice of mRNAs to be used as controls for analysis of the PCR reactions.

More specifically, quantitating PCR reactions, such as QRT-PCR reactions, typically is performed by one of two methods: comparison to a standard curve or comparison of Ct values. In the first of these methods, a standard curve of amplification products of a particular mRNA is made based on amplification of a series of different, known amounts of a pre-selected nucleic acid. Amplification results of reactions performed on a target nucleic acid are then compared to the standard curve to obtain a quantity, and that quantity can be extrapolated to an amount of the target in the original sample. While it is preferred to use an mRNA as the source for the standard curve, the stability of mRNA is known to affect the validity of such standard curves, and overcoming or minimizing this problem has proved to be difficult. To avoid the problems associated with using mRNA as a source for the standard curve, researchers have used DNA for generation of standard curves. While use of DNA overcomes the problems associated with use of mRNA, the mere fact that it avoids the problems creates yet another problem. That is, because DNA templates are relatively stable, and because amplification of DNA does not require a first-strand synthesis step (which can be inefficient and variable across samples or preparations), the standard curves produced from DNA sources often do not correlate accurately to the amount of mRNA in a test sample.

In the Ct comparison method for quantitating PCR products, expression of a housekeeping gene is used as a standard against which amplification of a target nucleic acid is compared. Often, in this method, a comparison of expression of the target nucleic acid under two different conditions is performed to determine changes in expression patterns. While this method avoids the problems associated with instability of RNA or use of DNA as a control that is seen when using the classical standard curve method, it requires selection of a housekeeping gene that is expressed at the same or nearly the same level in all tested samples and that can be amplified at the same or essentially the same efficiency as the target nucleic acid. Often, this selection process is tedious and time consuming. Not infrequently, a suitable housekeeping gene cannot be found, and the classical standard curve method must be used instead.

Recently, researchers have attempted to use controls that are amplified in the same PCR reaction mixture as the target sequence in an effort to quantitate PCR products and determine amounts of target nucleic acids in a sample. These controls are often transcripts of housekeeping genes or rRNA species. The control is added to the reaction mix and co-amplified with the target nucleic acid. Fluorescent probes specific for both are included in the mixture, and two amplification curves are obtained. The relative expression of the target nucleic acid with respect to the control is then determined. Using this technique, multiple samples of the same type (e.g., taken at different times during development of an organism or a disease or disorder) or multiple different types of samples can be compared for expression of a particular target, with reference back to the same control. Although adding a control to amplification reactions can be a useful alternative to other methods of quantitating expression levels, and can be a useful method for normalizing PCR reactions across samples, it does not allow one to determine absolute amounts of materials present in the amplification reaction mixture or in the original sample. Rather, the results are qualitative or semi-quantitative, giving an idea only of the amount of one nucleic acid (e.g., the target) in comparison to another (e.g., the control).

In view of the state of the art, it is apparent that new, more accurate and reproducible methods of quantitating PCR products, and the amounts of source materials for PCR reactions, are needed. In particular, methods and primers are needed to enable researchers, clinicians, and others to evaluate cell numbers or tissue amounts in samples comprising cell lysates. The methods would preferably be based on a reproducible, reliable standard that can be applied across sample types and be valid for numerous target nucleic acid amplification reactions and targets.

### SUMMARY OF THE INVENTION

The present invention addresses needs in the art by providing primers, compositions, methods, and kits for determining the amount of starting materials for nucleic amplification reactions, and for normalizing the amount of those materials across samples to enable accurate comparisons of amplification products. Current commercial technologies for quantitating amplification products and normalizing samples are based on amplification and detection of nucleic acids that are not necessarily present at the same amount in different samples, and thus often provide inaccurate benchmarks. The present invention overcomes this deficiency in the art by providing primers, methods, and unique target genomic nucleic acid sequences for use in accurate quantitation of the amount of genomic nucleic acid in a sample, an amount that can be compared, in embodiments, to a standard curve to determine the number of cells from which the nucleic acids originated or compared to another sample for relative quantitation of nucleic acids.

Isolation of nucleic acids is required for different gene expression analysis techniques (microarrays, PCR, QPCR and QRT-PCR). Using a unique lysis buffer composition disclosed in U.S. patent application Ser. No. 11/152,773, it is possible to perform QRT-PCR without RNA isolation. That method was initially developed for lysis of cells and amplification of mRNA from samples containing a known amount of cells. For tissues and cell lysates with unknown cell numbers, the method was adapted to evaluate cell numbers or tissue amounts in the lysate. Because cell lysates contain all cell components, including RNA, DNA, proteins, etc., the present inventors concluded that the least variable component should be used as a standard. Thus, it was concluded that the amount of genomic DNA present in the sample should be used for this purpose. That is, the present inventors realized that a suitable internal control for PCR reactions, such as QRT-PCR reactions, would be genomic DNA, which is not only present in each particular cell or tissue type in a known, finite amount, but is also relatively stable. Use of genomic DNA as an internal standard for quantitation and normalization of PCR samples is conceptually significantly different than other attempts in the art at providing “internal” controls for PCR reactions, such as through the addition to the reaction mixture of exogenous nucleic acids of known sequence.

By definition every species of prokaryotic or eukaryotic organism has a stable genome of a specific size. For example, the human genome is diploid, comprising 23 pairs of chromosomes, each pair comprising substantially identical sequences. There are twenty-two pairs of human autosomes and one pair of sex chromosomes (X and Y). It is well known that numerous definable sequences within each chromosome (whether it be a human chromosome or a genome or part of a genome from another organism) are identical with other sequences on that chromosome or on other chromosomes. However, unique sequences on each chromosome (or on a genome, if the organism comprises only one chromosome) can be identified. The present invention takes advantage of those unique sequences to provide a standard for the ultimate quantitation of genomic DNA present in samples. The quantitation of genomic DNA present in samples permits one not only to normalize the amount of sample tested (e.g., number of cells analyzed for mRNA expression), even across samples or tissues, but can provide a means for determining the quantity of a target nucleic acid species (e.g., a particular mRNA) in that sample.

Exemplary embodiments of the invention relate to human sequences. However, it is to be understood that the concepts and teachings of the invention can be applied to any and all organisms that have one or more unique sequences within their genomes. In exemplary embodiments of the present invention, unique human genomic DNA sequences have been identified on each human chromosome. PCR primers have been designed and synthesized to amplify these unique sequences. Using these PCR primers and known quantities of human genomic DNA, a standard curve was generated, which correlates Ct values with the quantity of known genomic DNA. The Ct values for samples containing unknown quantities of genomic DNA can then be compared with the Ct values from the standard curve to establish genomic DNA quantity in samples destined for analysis, such as for QRT-PCR analysis. Running this control QPCR reaction simultaneously with QRT-PCR (either in the one or two tube reaction format) allows normalizing of the amount of nucleic acid being amplified. Alternatively, a sample of known identity comprising a known unique DNA sequence may be used as a relative standard against which other samples, or select nucleic acids such as mRNA, may be compared to provide semi-quantitative or qualitative comparisons of two or more samples or expression products.

In a first aspect, the invention provides nucleic acids for amplifying and detecting unique genomic sequences in the genome of a cell of interest. In embodiments, the unique genomic sequences are present on one or more chromosomes of a cell of interest. Preferably, primers are provided in pairs such that amplification of the target unique genomic sequence can be performed. Any primer or probe, primer pair, or primer/probe combinations that amplify, and preferably detect, all or part of a unique genomic sequence are contemplated by the present invention. Primers can be provided that are specific for unique sequences on different chromosomes of cells, including cells from various mammals, avians, amphibians, reptiles, insects, fungi (such as yeast), plants, and prokaryotes (including archaea).

In a second aspect, the invention provides compositions comprising one or more primer of the invention, or one or more amplification product of the primer(s). The compositions can be liquids (e.g., stock solutions or amplification reactions) or solids (e.g., lyophilized purified primers) and can comprise the primer(s) in any amount or concentration. In exemplary embodiments, the compositions comprise some or all of the components necessary for amplification of nucleic acids, such as the components necessary for performing a PCR technique (e.g., RT-PCR or QRT-PCR).

In a third aspect, the invention provides methods for quantitating the amount of sample provided in an amplification reaction. In general, the methods comprise providing a test sample containing genomic nucleic acid, amplifying one or more unique genomic sequences present in the genomic nucleic acid, and comparing the amplification profile (e.g., the Ct value) to a standard curve of amplification profiles obtained from reactions performed on reference samples from known numbers of original cells, and determining the number of cells from which the test sample genomic nucleic acids originated. In embodiments, the methods further comprise performing an amplification reaction with primers that are specific for a nucleic acid sequence of interest (i.e., a target expression sequence). According to the present methods, when target expression sequences are amplified, their amplification profiles can be accurately compared to amplification profiles of other samples by normalizing the amount of starting target expression sequences between samples based on the number of original cells from which each sample was obtained.

According to the invention, DNA can be also used as the normalizer for the relative or comparative quantification of gene expression (e.g., RNA). In order to compare the gene expression level in two or more samples with unknown (or different) input material, the DNA can be used as an internal normalizer instead of housekeeping genes (like B2M, GAPDH) or in addition to the housekeeping genes. The Ct values obtained from QPCR reaction (DNA) can be used for the normalization of Ct values obtained from QRT-PCR reaction (see FIG. 30 for example, discussed in more detail below).

In a fourth aspect, the invention provides kits. In general, the kits contain some or all of the components necessary to practice a method of the invention. Thus, for example, the kits may contain one or more primer or one or more composition of the invention. Likewise, the kits may contain multiple primers, or sets of primers, for amplification of unique genomic sequences or for amplification of target expression sequences. In preferred embodiments, a kit of the invention comprises any primer pair that amplifies part or all of a unique genomic sequence from a pre-selected genome.

### BRIEF DESCRIPTION OF THE DRAWINGS

The accompanying drawings, which are incorporated in and constitute a part of this specification, illustrate several embodiments of the invention and, together with the written description, serve to explain certain principles or details of embodiments of the invention.

FIG. 1A depicts amplification plots of HeLa gDNA using primer set #2 at 10-fold dilutions from 10 ng gDNA to 0.01 ng gDNA.

FIG. 1B depicts dissociation curves of the amplification products from FIG. 1A.

FIG. 2A depicts amplification plots of HeLa gDNA using primer set #3 at 10-fold dilutions from 10 ng gDNA to 0.01 ng gDNA.

FIG. 2B depicts dissociation curves of the amplification products from FIG. 2A.

FIG. 3A depicts amplification plots of HeLa gDNA using primer set #8 at 10-fold dilutions from 10 ng gDNA to 0.01 ng gDNA.

FIG. 3B depicts dissociation curves of the amplification products from FIG. 3A.

FIG. 4A depicts amplification plots of HeLa gDNA using primer set #9 at 10-fold dilutions from 10 ng gDNA to 0.01 ng gDNA.

FIG. 4B depicts dissociation curves of the amplification products from FIG. 4A.

FIG. 5A depicts amplification plots of HeLa gDNA using primer set #10 at 10-fold dilutions from 10 ng gDNA to 0.01 ng gDNA.

FIG. 5B depicts dissociation curves of the amplification products from FIG. 5A.

FIG. 6 depicts a No Template Control (NTC) of the amplification reaction for ten primer sets for amplification of unique sequences. No primer-dimer amplification was observed.

FIG. 7A depicts the amplification profiles for eight human cell type gDNA samples (1 ng of each) using primer set #1 (SEQ ID NO:1 and SEQ ID NO:2).

FIG. 7B depicts the dissociation curves of the amplification products produced in the amplification reactions depicted in FIG. 7A.

FIG. 8A depicts the amplification profiles for eight human cell type gDNA samples (1 ng of each) using primer set #2 (SEQ ID NO:3 and SEQ ID NO:4).

FIG. 8B depicts the dissociation curves of the amplification products produced in the amplification reactions depicted in FIG. 8A.

FIG. 9A depicts the amplification profiles for eight human cell type gDNA samples (1 ng of each) using primer set #3 (SEQ ID NO:5 and SEQ ID NO:6).

FIG. 9B depicts the dissociation curves of the amplification products produced in the amplification reactions depicted in FIG. 9A.

FIG. 10A depicts the amplification profiles for eight human cell type gDNA samples (1 ng of each) using primer set #4 (SEQ ID NO:7 and SEQ ID NO:8).

FIG. 10B depicts the dissociation curves of the amplification products produced in the amplification reactions depicted in FIG. 10A.

FIG. 11A depicts the amplification profiles for eight human cell type gDNA samples (1 ng of each) using primer set #5 (SEQ ID NO:9 and SEQ ID NO:10).

FIG. 11B depicts the dissociation curves of the amplification products produced in the amplification reactions depicted in FIG. 11A.

FIG. 12A depicts the amplification profiles for eight human cell type gDNA samples (1 ng of each) using primer set #6 (SEQ ID NO:11 and SEQ ID NO:12).

FIG. 12B depicts the dissociation curves of the amplification products produced in the amplification reactions depicted in FIG. 12A.

FIG. 13A depicts the amplification profiles for eight human cell type gDNA samples (1 ng of each) using primer set #7 (SEQ ID NO:13 and SEQ ID NO:14).

FIG. 13B depicts the dissociation curves of the amplification products produced in the amplification reactions depicted in FIG. 13A.

FIG. 14A depicts the amplification profiles for eight human cell type gDNA samples (1 ng of each) using primer set #8 (SEQ ID NO:15 and SEQ ID NO:16).

FIG. 14B depicts the dissociation curves of the amplification products produced in the amplification reactions depicted in FIG. 14A.

FIG. 15A depicts the amplification profiles for eight human cell type gDNA samples (1 ng of each) using primer set #9 (SEQ ID NO:17 and SEQ ID NO:18).

FIG. 15B depicts the dissociation curves of the amplification products produced in the amplification reactions depicted in FIG. 15A.

FIG. 16A depicts the amplification profiles for eight human cell type gDNA samples (1 ng of each) using primer set #10 (SEQ ID NO:19 and SEQ ID NO:20).

FIG. 16B depicts the dissociation curves of the amplification products produced in the amplification reactions depicted in FIG. 16A.

FIG. 17A depicts amplification plots of HeLa gDNA using primer set # 10 and 40 pg to 5 ng of gDNA.

FIG. 17B depicts the standard curve created from the data in FIG. 17A.

FIG. 18A depicts amplification plots of cell lysates created in a buffer comprising 5 mM TCEP, 1% Triton X-100, pH 2.5.

FIG. 18B depicts amplification plots of cell lysates created using the Ambion Cells To Signal II™ buffer.

FIG. 19A depicts amplification plots of cell lysates created in a buffer comprising 5 mM TCEP, 1% Triton X-100, pH 2.5.

FIG. 19B depicts amplification plots of cell lysates created using the Ambion Cells To Signal II™ buffer.

FIG. 20A depicts standard curves created from the data presented in FIGS. 18A and 18B.

FIG. 20B depicts standard curves created from the data presented in FIGS. 19A and 19B.

FIG. 21 presents a summation of the data presented in FIGS. 18A through 20B.

FIG. 22 depicts standard curves for amplification of target sequences using QPCR and QRT-PCR.

FIG. 23 depicts the standard curve and amplification plots for QPCR amplification with HeLa cell lysates using primer set 10 (SEQ ID NO:19 and SEQ ID NO:20) and a buffer of the invention.

FIG. 24 depicts the standard curve and amplification plots for QRT-PCR amplification with HeLa cell lysates using RNA-specific B2M TaqMan® primers and probes and a buffer of the invention.

FIG. 25 presents a summation of the data presented in FIGS. 23 and 24.

FIG. 26 depicts the standard curve and amplification plots for QPCR amplification of human liver tissue lysate with primer set 10 (SEQ ID NO:19 and SEQ ID NO:20).

FIG. 27 depicts the standard curve and amplification plots for QRT-PCR amplification of human liver tissue lysate with RNA-specific B2M TaqMan® primers and probes.

FIG. 28 presents a summation of the data presented in FIGS. 26 and 27.

FIG. 29 compares QPCR and QRT-PCR amplification reactions at four different HeLa cell concentrations, comparing DNA-specific primer set 10 (SEQ ID NO:19 and SEQ ID NO:20) in QPCR to BAX, USP7, and B2M RNA-specific TaqMan® primers and probes in one-step QRT-PCR.

FIG. 30 presents comparative quantification of BAX gene expression in HeLa cells using B2M (QRT-PCR) or DNA (QPCR) as the normalizer for BAX amplification, demonstrating that single-copy gDNA can be successfully used as the normalizer in comparative quantification analysis of gene expression.

### DETAILED DESCRIPTION OF VARIOUS EMBODIMENTS OF THE INVENTION

Reference will now be made in detail to various exemplary embodiments of the invention. The following detailed description is provided to more fully explain various exemplary embodiments of the invention, and is not intended to limit the scope of the invention in any way.

The present invention provides primers, compositions, methods, and kits for determining the amount of starting materials for nucleic amplification reactions, and for normalizing the amount of those materials across samples to enable accurate comparisons of amplification products. In particular, it provides primers, compositions, methods, and kits for determining numbers of cells or tissue amounts from which cell lysates originated. The invention can also be useful for evaluating numbers of cells or amounts of nucleic acids in samples comprising isolated or partially isolated nucleic acids. Furthermore, the invention can be used for normalization of mRNA and for karyotyping. Current commercial technologies for normalizing samples are based on amplification and detection of nucleic acids that are not necessarily present in the same amount in the different samples being tested, and thus often provide inaccurate results. The present invention overcomes this deficiency in the art by providing primers, methods, and unique target genomic nucleic acid sequences for use in accurate quantitation of the amount of genomic nucleic acid in a sample, an amount that can be compared to a standard curve to determine the number of cells from which the nucleic acids originated. With the knowledge of the total number of cells from which a particular amplification product is produced, one can determine, with a high degree of assurance, the relative abundance of various expression products among different samples.

In a first aspect, the invention provides nucleic acids. In general, the nucleic acids are primers and probes for amplifying and detecting unique genomic sequences within the genome of a cell of interest, or are nucleic acids comprising unique sequences within a genome of an organism. Primers are generally of two types: those that are specific for unique genomic sequences present in the genome of a particular cell, and that can be used to amplify those unique genomic sequences; and those that are specific for a target sequence other than a unique genomic sequence (e.g., an mRNA species of interest). Probes are generally designed to identify unique genomic sequences or a target sequence other than a unique genomic sequence, and are typically designed in conjunction with the primers for amplification of the unique genomic sequence or in conjunction with the primers for the target sequence. In contrast, nucleic acids comprising unique genomic sequences are relatively long nucleic acids that are provided either as pre-formed units or are synthesized using the primers and methods of the invention. Pre-formed nucleic acids such as these can be used as controls for amplification reactions that specifically amplify unique genomic sequences, whereas these types of nucleic acids that are synthesized during practice of a method of the invention can be used to determine the quantity of cellular material in a reaction or in a sample from which the reaction materials originated.

When applied to organisms with a single chromosome, the primers for unique genomic sequences are designed to be specific for one or more unique sequences on that chromosome, different primers being designed specifically for each different unique sequence present on the chromosome, if more than one unique sequence is present. When applied to organisms with multiple chromosomes, the primers are designed to be specific for one or more unique sequences on one or more of each chromosome, different primers being designed specifically for each different unique sequence present on each chromosome selected, if more than one unique sequence is present. Thus, for organisms containing two or more chromosomes, two or more primers, and preferably two or more sets of primers (a set comprising at least two primers, an upstream and a downstream primer) can be designed, each primer or set of primers being designed to amplify a different unique genomic sequence. In embodiments, different primer sets are designed for different unique genomic sequences on different chromosomes (e.g., one primer set for a unique sequence on chromosome 1, another primer set for a different unique sequence on chromosome 2, etc.).

Primers for amplification of target sequences (e.g., mRNA sequences of interest) can be designed based on the sequence of the target sequence, in accordance with standard procedures and considerations for design of PCR primers. Design and synthesis of such primers is well within the abilities of those of skill in the art, and the details need not be discussed here.

It is to be noted that primers can function as both primers for amplification of a unique genomic sequence or a target sequence and as a generator of a signal for detection and/or monitoring of an amplification reaction. Thus, in embodiments, the primers are unlabeled, while in other embodiments, the primers are labeled, such as with a fluorescent moiety. Labeled primers can be of any type, including those that are typically used in QPCR reactions, such as Scorpions, Molecular Beacons, Sunrise primers, and the like.

Probes may be provided in addition to primers. Probes that can be used for detection of amplification of the unique genomic sequences (e.g., TaqMan® probes) can be designed to hybridize to a sequence between the two amplification primers, preferably within 5-15 bases of one of the primer binding sites. Design and synthesis of such probes is well within the abilities of those of skill in the art, and the details need not be discussed here. Typically, probes are present in reaction mixtures in conjunction with primers or sets of primers for a particular amplification reaction, whether it be an amplification of a unique genomic sequence or a target sequence. However, probes may be provided as separate components, which are separate from the primer(s) or other components of a reaction mixture.

The primers and probes are designed to have the typical size for primers and probes for use in PCR reactions. In general, the primers are relatively short (about 7-40 bases in length) oligonucleotides, while the probes (e.g., TaqMan® probes) are from about 30 to about 150 bases in length. The primers and probes are designed through a process that includes identification of unique sequences within the genome of an organism or identification of a suitable sequence on a target nucleic acid, designing short oligonucleotides to amplify and/or detect those sequences, and synthesizing the oligonucleotides. Protocols for synthesis of oligonucleotides are now commonly known to those of skill in the art. Any suitable protocol may be used in synthesizing the primers and probes of the invention.

Preferably, primers are provided in pairs such that amplification of the target unique genomic sequence can be performed. The primers may be designed using standard considerations for PCR primers. Where probes are used, the probes may be designed using standard consideration for QPCR probes, including, but not limited to, the following: the C content should be higher than the G content, and the 5′-end is not G. In addition, where both primers and a probe are to be used, the following additional characteristics may be taken into consideration when designing the primers and probe: the probe melting temperature should be higher than the primer melting temperatures, and the distance between the 3′-end of one primer and the 5′-end of the probe should be less than 8 nucleotides. Of course, various considerations and characteristics for primers and probes will be applicable to certain primers and probes, but not others. One of skill in the art is well aware of these considerations and characteristics, and may select among them to provide suitable primers and probes according to the invention without undue or excessive experimentation.

Primers according to the invention are typically used in pairs to amplify unique genomic sequences. Thus, according to embodiments of the invention, any primer pair that amplifies a unique genomic sequence from an organism is suitable for use in the invention, and in particular for use in methods of the invention (discussed in detail below). Any number of primer pairs may be devised by those of skill in the art without undue experimentation, now that a good portion or the entire genomic sequence of so many organisms is known. Within the context of the invention, the precise sequence of any particular primers is not critical. Rather, the concept of the invention may be applied to any primers that are specific for unique genomic sequences.

Thus, in embodiments, primers that comprise sequences found in any of the sequences disclosed herein, including the following, or are complementary to any of the sequences disclosed herein, are suitable for use in the invention:

Human Chromosome 3:
GGTGAAGATAATGAAAGTCATTGGTATTTCTTAGATTTTTCATGCTCAAA
AGTCACAAGGGACTTTGTAAACTGAATCTGATTGATGATAATTGCAACCT
AAAAGAAGAGGATTTGAATTTCTGAAGTTTATGCCAGAACTGACATCTAT
TCTGATTCCTGTTCCAATCAGTCCTTCATTAAAAGTTGCCTGTTTCTGCC
AGTATGCTCTTACTGTTA
Human Chromosome 5:
ACACACATAGTGGTTTATGAAGAACCTGTCATAAACCTAAACGATAGCAC
CAATTAGAAATGAGCTTCCATTGATATATTTCAGATCATTTGCCCTTACC
CTTTTCTGACTTTCTGATTCTTTAAACTCATGAACATGGTTGAGTGTATC
ATCGGGTGATGAATAGCTAGAGATGACTGAGGCCAACAGACTATATATTA
Human Chromosome 8:
TGTGATCTAAAGGGTGTGTGTATAATCCAGAAAGCTACTAGCTGCATAGT
CTTTTCTTACAAAGTATTTAGCCTTCACCTTTCAAGATTTCCTTCCTCTC
AATATTTGGAGAGGACAGAAGGATAGATATTCTAATACTATTTTCATATT
GGTCTGTGCTTTTGAGATCACCATGCTCCTTTGAAAATACTGCCGGGCGC
CCTGGCCTCTGTAGAATAGCAGTGCCAGGGAGGCCTTTATCTCCTGATGG
CTCATACTAAG
Human Chromosome 9:
TATAAGAAACTACTAAGCACCCAAAGGAACATCAAATACCCAGTGTCCTG
CAAATGACTGTAGGATAGGTAAGAGTAGCTAATGGTGATATTAATGCTGT
ATAATACAATTTAAAATTAGTATCTCTCCTCTTTCCATCACCTAAATTGG
CTTACTTTCCGTATTTAAAATTCACAATAAGTGGCATCAACATGAATGGA
GTTGGCCTGTTGAGTCACTTAGACTCCTTTCTT
Human Chromosome 15:
AAGTCTATATCTCCAAACAAGTCCTCATAGACTATTTAGTCCTAATTCCT
ACTGAGCATTTTCCCCATCAGCTAAACTTAACACTGTAAGCATCATTTTC
ACCTCCATCCTAGATGCTGCTCTCTTTCTAAGCTGTCTTACTTCTACCAC
TATCATTCCTTCAGTCTTTAAGGAGAGCATTTATAGTAATTTTTGACTCT
TTCCTTTTCCTTAACCTTTAGGCAGTTATCAAGGCTTACTGTTTTTACTT
TGAACATCATTGCTATCCTATTC
Human Chromosome 20:
GCCTATTTCTCCTGGTAGTTTAGAAATATAATTACCTGGATAAGACCACC
AACTAATTTCACTTTCACCGTCATTCAGTAAATCTCAGAAATATAAGCAA
AGAACAATCTTGGACAAGAGAAAAGAAGAACCTGATCTCTTTTCCAGCCC
TATGACTCACTGAAGAAACCAGGAATATGCCACGTGTTCTCTTTCTGCTG
CAAGGGTTGCTGTGAAATAACCTCATTTAAGCTGTGTTGTACA

Of course, numerous other sequences for primers can be identified and used according to the invention without the specific sequences needing to be disclosed here. The important concept to be recognized is that any primer pair (or more) that can be used to amplify and preferably detect a unique genomic sequence from any organism can be used according to the invention, and the invention is not and should not be considered to be limited to any particular specific primers or primer pairs.

Primers are provided that are specific for unique sequences within the genome of any type of cell. Among the cells for which primers and probes can be designed include cells from various mammals, birds, amphibians, reptiles, insects, fungi, and prokaryotes. Indeed, unique sequences within virus genomes, particularly those that integrate into a host cell's genome in a known number, can be used for design of primers and probes. As discussed below, the methods of the invention are suitable for use on samples from any cells or tissues, and are not limited to sources listed herein.

Identification of unique sequences within an organisms genome can be accomplished in many ways. The complete, or substantially complete, genome sequences of numerous organisms are now available, publicly or through private vendors, and any of these may be used for identification of unique genomic regions and design of primers and probes for detecting unique sequences. In one particular embodiment, a computer can be used to screen the entire sequence of a particular genome to identify regions with unique sequences. Suitable sub-sequences within these unique genomic sequences can be identified by computer analysis to provide acceptable primer and probe sequences for amplification of the unique genomic sequences. In addition, certain genomic regions of organisms have been found through experimentation, or can be found through experimentation, to be unique within the context of that organism's genome. Such experimentally-identified regions can be used for design of primers and probes as well. Although not required, primer pairs and probes can be tested for specificity and efficacy in amplifying the genomic sequences. Among non-limiting exemplary primer sequences, six may be noted at this point:

For Human Chromosome 3 (primer set 26)
up- GGTGAAGATAATGAAAGTCATTGGTAT
down- TAACAGTAAGAGCATACTGGCAGAAAC
For Human Chromosome 5 (primer set 18)
up- ACACACATAGTGGTTTATGAAGAACCT
down- TAATATATAGTCTGTTGGCCTCAGTCA
For Human Chromosome 8 (primer set 9)
SEQ. ID NO: 17 and 18
For Human Chromosome 9 (primer set 10)
SEQ. ID NO: 19 and 20
For Human Chromosome 15 (primer set 3)
SEQ. ID NO: 5 and 6
For Human Chromosome 20 (primer set 2)
SEQ. ID NO: 3 and 4.

One feature of the primers of the invention is that they are specific for unique sequences within the genome of the cell being analyzed. Thus, amplification profiles generated from the use of the primers, and optionally probes as well, are known to relate back to one copy (if the source cell is haploid for that sequence) or two copies (if the source cell is diploid for that sequence) per original source cell. In this way, the primers and probes may be used to generate not only standard curves of known amounts of original cell source material, but as internal controls for normalization and quantitation of samples originating from an unknown number of cells. Likewise, they can be used for karyotyping of cells. When used in conjunction with amplification of a target sequence (e.g., an mRNA), the primers and optional probes can provide a fast, reliable, reproducible way to determine the amount of starting material being amplified, and enable the practitioner to normalize samples among and across cell types, tissue types, and sample time points. In effect, the primers and optional probes provide an internal control for any type of sample, including but not limited to cells or cell lysates, to be tested for the presence and amount of a particular target sequence, and permit the practitioner to draw conclusions regarding the amount of cellular material in the original sample and the relative amount of one or more target sequences with regard to other sequences or with regard to the same sequence present in other tissues or at other points in time during development of an organism, a disease or disorder, or a treatment regimen.

According to the present invention, primers and probes can be designed to be specific for one or more unique sequences on a cell genome. In some embodiments, multiple primer sets or primer and probe sets are provided, where each primer set or primer and probe set are specific for a different unique sequence within the source cell's genome. For example, in embodiments, two or more primer sets are provided in which each primer set comprises two primers that are specific for a unique sequence on a single chromosome, and in which each primer set is specific for a different chromosome within the genome of the source cell. Thus, in embodiments, a primer set that is specific for a unique sequence on human chromosome 9 and a primer set that is specific for a unique sequence on human chromosome 20 are both provided. In other embodiments, an additional primer set, which is specific for a unique sequence on human chromosome 15, is provided. Various other combinations of primer sets can immediately be envisioned by those of skill in the art. Accordingly, each permutation of primer sets and primer and probe sets need not be specifically listed here. It is sufficient that those of skill in the art recognize that all combinations of two or more primer sets or primer and probe sets are envisioned by the present invention.

Table 1, below, lists exemplary primer sets for use in amplifying unique sequences from certain human chromosomes. These particular sequences are provided as examples of primers that can be used according to the invention, and are not intended as a complete or exclusive listing of primer sequences for human cells.

TABLE 1

Oligonucleotide Primer Sequences for Human
Chromosomes Using SYBR ® Green Dye

| | | | | |
|---|---|---|---|---|
| Primer | | | | |
| Name | | | Amplicon | SEQ |
| (human | | Tm | Size | ID |
| x-some #) | Sequence | (° C.) | (bp) | NO |

| | | | | |
|---|---|---|---|---|
| 1 UP | 5′-aacagaaatctggatgtgttattaagg-3′ | 60.1 | 215 | 1 |
| (7) | | | | |
| 1S DOWN | 5′-agaatagataagatgcagtcaccactt-3′ | 60.1 | | 2 |
| (7) | | | | |
| 2 UP | 5′-gcctatttctcctggtagtttagaaat-3′ | 59.9 | 244 | 3 |
| (20) | | | | |
| 2S DOWN | 5′-ctgtacaacacagcttaaatgaggtta-3′ | 60.1 | | 4 |
| (20) | | | | |
| 3 UP | 5′-aagtctatatctccaaacaagtcctca-3′ | 60.0 | 273 | 5 |
| (15) | | | | |
| 3S DOWN | 5′-gaataggatagcaatgatgttcaaagt-3′ | 60.2 | | 6 |
| (15) | | | | |
| 4 UP | 5′-tcttgttcttgtcagttctctaaatca-3′ | 59.9 | 241 | 7 |
| (3) | | | | |
| 4S DOWN | 5′-ttgttatatacctgcattcaatcagaa-3′ | 60.2 | | 8 |
| (3) | | | | |
| 5 UP | 5′-aactcctaactgataaaggttctggat-3′ | 60.2 | 220 | 9 |
| (9) | | | | |
| 5S DOWN | 5′-tgagaacacaaagagttgtttctaatg-3′ | 60.1 | | 10 |
| (9) | | | | |
| 6 UP | 5′-aatgaatattcttcttacccacgtaga-3′ | 59.8 | 254 | 11 |
| (18) | | | | |
| 6S DOWN | 5′-ctgcaaatttaactatcaaatgacaaa-3′ | 59.9 | | 12 |
| (18) | | | | |
| 7 UP | 5′-cttgaatttctcttctgtggtctaatc-3′ | 60.1 | 251 | 13 |
| (11) | | | | |
| 7S DOWN | 5′-tcccttaatataaagtacaaattgcgt-3′ | 59.7 | | 14 |
| 8 UP | 5′-aaattctcctagcattcaaacctactt-3′ | 60.3 | 284 | 15 |
| (4) | | | | |
| 8S DOWN | 5′-gttgacctttcttatggttgcttatag-3′ | 59.9 | | 16 |
| (4) | | | | |
| 9 UP | 5′-tgtgatctaaagggtgtgtgtataatc-3′ | 59.6 | 261 | 17 |
| (8) | | | | |
| 9S DOWN | 5′-cttagtatgagccatcaggagataaag-3′ | 60.1 | | 18 |
| (8) | | | | |
| 10 UP | 5′-tataagaaactactaagcacccaaagg-3′ | 59.6 | 233 | 19 |
| (9) | | | | |
| 10S DOWN | 5′-aagaaaggagtctaagtgactcaacag-3′ | 59.9 | | 20 |
| (9) | | | | |

Table 2, below, lists exemplary primers and probes for use in detecting amplified unique sequences from certain human chromosomes, which can be used in TaqMan® assays. The particular sequences of Tables 1 and 2 are provided as examples of primers and probes that can be used according to the invention, and are not intended as a complete or exclusive listing of primer and probe sequences for human cells.

TABLE 2

Primers and Probes for Detection of Amplification
of Unique Human Genomic Sequences Using the
TaqMan ® Assay

| | | | |
|---|---|---|---|
| Primer | | | |
| or Probe | | | |
| Name | | | SEQ |
| (human | | Tm | ID |
| x-some #) | Sequence | (° C.) | NO |

| | | | |
|---|---|---|---|
| 1 UP (7) | 5′-AACAGAAATCTGGATGTGTTATTAAGG-3′ | 60.1 | 1 |
| 1T DOWN | 5′-GTTTGTACAGACTCCGTAAGATTTGTT-3′ | 60.2 | 21 |
| (7) | | | |
| 1TP (7) | 5′-AATGACCAGTACAATTTCCCTTTATCACTAAAAA-3′ | 66.1 | 22 |
| 2 UP (20) | 5′-GCCTATTTCTCCTGGTAGTTTAGAAAT-3′ | 59.9 | 3 |
| 2T DOWN | 5′-ATATTTCTGAGATTTACTGAATGACGG-3′ | 60.2 | 23 |
| (20) | | | |
| 2TP (7) | 5′-ACCTGGATAAGACCACCAACTAATTTCACTTTCA-3′ | 69.9 | 24 |
| 3 UP (15) | 5′-AAGTCTATATCTCCAAACAAGTCCTCA-3′ | 60.0 | 5 |
| 3T DOWN | 5′-TGCTTACAGTGTTAAGTTTAGCTGATG-3′ | 60.3 | 25 |
| (15) | | | |
| 3TP (15) | 5′-CTATTTAGTCCTAATTCCTACTGAGCATTTTCCC-3′ | 66.4 | 26 |
| 4 UP (3) | 5′-TCTTGTTCTTGTCAGTTCTCTAAATCA-3′ | 59.9 | 7 |
| 4T DOWN | 5′-ATATCTAAGAGATTCTTGTGTGATGCC-3′ | 60.3 | 27 |
| (3) | | | |
| 4TP (3) | 5′-AAGGAAACCCGTTTTCTCAGCCTCAATCTTTC-3′ | 72.7 | 28 |
| 5 UP (9) | 5′-AACTCCTAACTGATAAAGGTTCTGGAT-3′ | 60.2 | 9 |
| 5T DOWN | 5′-ATGTGCCAAAGTAATTTAGAATTGAAG-3′ | 60.2 | 29 |
| (9) | | | |
| 5TP (9) | 5′-AACTTATGAATGTCCCAATAGTGACCCATTTTAA-3′ | 68.0 | 30 |
| 6 UP (18) | 5′-AATGAATATTCTTCTTACCCACGTAGA-3′ | 59.8 | 11 |
| 6T DOWN | 5′-ACTCATCTATCTAATTACTTCGCCCTT-3′ | 60.3 | 31 |
| (18) | | | |
| 6TP (18) | 5′-TACAAGCATAGAAACAATACCCATACACTCCTCA-3′ | 68.4 | 32 |
| 7 UP (11) | 5′-CTTGAATTTCTCTTCTGTGGTCTAATC-3′ | 60.1 | 13 |
| 7T DOWN | 5′-TTTCCAATGCAGTCAGATAAGAAATA-3′ | 60.4 | 33 |
| (11) | | | |
| 7TP (11) | 5′-ACAAATATAAAAGCCTGCATTCCTTCTATTCATT-3′ | 66.8 | 34 |
| 8 UP (4) | 5′-AAATTCTCCTAGCATTCAAACCTACTT-3′ | 60.3 | 15 |
| 8T DOWN | 5′-GGCCATCAATAAATATCAACTTAGAAA-3′ | 60.0 | 35 |
| (4) | | | |
| 8TP (4) | 5′-CCCAGCACTCTTCCAAGCACTGTATAAATCATAT-3′ | 70.2 | 36 |
| 9 UP (8) | 5′-TGTGATCTAAAGGGTGTGTGTATAATC-3′ | 59.6 | 17 |
| 9T DOWN | 5′-AATCTTGAAAGGTGAAGGCTAAATACT-3′ | 60.3 | 37 |
| (8) | | | |
| 9TP (8) | 5′-CAGAAAGCTACTAGCTGCATAGTCTTTTCTTACAA-3′ | 66.5 | 38 |
| 9 UP (9) | 5′-TATAAGAAACTACTAAGCACCCAAAGG-3′ | 59.6 | 19 |
| 9T DOWN | 5′-TCACCATTAGCTACTCTTACCTATCCT-3′ | 59.3 | 39 |
| (9) | | | |
| 9TP (9) | 5′-AACATCAAATACCCAGTGTCCTGCAAATGACTGT-3′ | 72.8 | 40 |

In a second aspect, the invention provides compositions comprising one or more nucleic acid. Typically, the compositions comprise one or more primer or probe of the invention, or one or more nucleic acid comprising the sequence of an amplification product of the primer(s). Although the compositions may be any composition known to those of skill in the art, typically, the compositions comprise purified primer(s) and/or probe(s), purified nucleic acids comprising unique genomic sequences, or amplification reaction mixtures, such as QRT-PCR reaction mixtures.

In general, the compositions comprise one or more component that is useful for practicing at least one embodiment of the method of the invention, or is produced through practice of at least one embodiment of the method of the invention. Accordingly, the compositions can be liquids or solids, and can comprise the nucleic acid in any amount or concentration. Although not so limited, typically liquid compositions of the invention are aqueous compositions, such as solutions or mixtures comprising one or more nucleic acid of the invention (e.g., primer, probe, amplification product, amplification substrate). Liquid compositions may comprise one or more organic solvent, either as the sole component or in addition to water. Furthermore, liquid compositions may comprise dyes, including reference dyes such as ROX, or other components of a signal generation system, which will typically be used to detect the presence of amplification products of either unique genomic sequences or target sequences (e.g., mRNA).

Furthermore, the compositions may be present in any suitable environment, including, but not limited to, reaction vessels (e.g., microfuge tubes, PCR tubes, plastic multi-well plates, microarrays), vials, ampules, bottles, bags, and the like. In situations where a composition comprises a single substance according to the invention, the composition will typically comprise some other substance, such as water or an aqueous solution, one or more salts, buffering agents, and/or biological material. Compositions of the invention can comprise one or more of the other components of the invention, in any ratio or form. Likewise, they can comprise some or all of the reagents or molecules necessary for amplification of target nucleic acids or unique genomic sequences, or both. Thus, the compositions may comprise ATP, magnesium or manganese salts, nucleoside triphosphates, and the like. They also may comprise some or all of the components necessary for generation of a signal from a labeled nucleic acid of the invention.

A composition of the invention may comprise one or more primer oligonucleotides. The primer(s) may be any primer(s) according to the invention, in any number of copies, any amount, or any concentration. Typically, the primer(s) will be primers for amplification of one or more unique genomic sequence. The primer(s) may be provided as the major component of the composition, such as in a purified or partially purified state, or may be a minor component. The practitioner can easily determine suitable amounts and concentrations based on the particular use envisioned at the time. Amounts can be relatively high, on the order of micrograms (ug), when the composition comprises a purified primer as the major component, such as in a lyophilized powder of the primer or a stock solution of the primer. In contrast, amounts can be relatively low, on the order of picograms (pg) or nanograms (ng), when the composition is a reaction mixture for amplification of a unique genomic sequence, a target sequence, or both, such as in a PCR reaction.

Thus, a composition according to the invention may comprise a single primer. On the other hand, it may comprise two or more primers, each of which having a different sequence, or having a different label or capability for labeling, from all others in the composition. Non-limiting examples of compositions of the invention include compositions comprising one or more primers, and a sample containing or suspected of containing a unique sequence present in a genome of a cell from which the sample originated. In embodiments, it also comprises a target nucleic acid of interest, such as an mRNA of interest. In embodiments, it also comprises a probe for detection of amplification of the unique genomic sequence. In embodiments, it comprises a probe for detection of amplification of the mRNA of interest. Other non-limiting examples include compositions comprising one or more primers that specifically amplify a pre-selected unique genomic sequence, a sample containing or suspected of containing the pre-selected unique genomic sequence and an mRNA of interest, and one or more primers that specifically amplify the mRNA of interest or a sequence found within the mRNA of interest. Yet other non-limiting examples include compositions comprising one or more of the components listed above and at least one polymerase, which is capable under appropriate conditions of catalyzing the polymerization of at least one of the primers in the composition to form a polynucleotide. In embodiments, the compositions comprise at least one reverse transcriptase. In embodiments, the compositions comprise at least one thermostable DNA polymerase. In particular embodiments, the compositions comprise two thermostable DNA polymerases. In embodiments, the compositions comprise both at least one reverse transcriptase and at least one thermostable DNA polymerase. In certain embodiments, the compositions comprise labels or members of a labeling system.

Thus, in embodiments, the composition comprises at least one oligonucleotide primer, each of these primers having a sequence comprising the sequence of any of SEQ ID NO:1 through SEQ ID NO:20, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:27, and SEQ ID NO:29, another sequence disclosed herein or its complement, or a portion of a sequence disclosed herein or its complement. For example, the composition can comprise a primer having a sequence comprising SEQ ID NO:1 and a primer having a sequence comprising SEQ ID NO:2. The composition can comprise a primer having a sequence comprising SEQ ID NO:3 and a primer having a sequence comprising SEQ ID NO:4, SEQ ID NO:5 and SEQ ID NO:6, SEQ ID NO:7 and SEQ ID NO:8, SEQ ID NO:9 and SEQ ID NO:10, SEQ ID NO:11 and SEQ ID NO:12, SEQ ID NO:13 and SEQ ID NO:14, SEQ ID NO:15 and SEQ ID NO:16, SEQ ID NO:17 and SEQ ID NO:18, SEQ ID NO:19 and SEQ ID NO:20, or combinations of two or more of these primer sets. Likewise, the composition can comprise a primer having a sequence comprising SEQ ID NO:1 and SEQ ID NO:21, SEQ ID NO:3 and SEQ ID NO:23, SEQ ID NO:5 and SEQ ID NO:25, SEQ ID NO:7 and SEQ ID NO:27, SEQ ID NO:9 and SEQ ID NO:29, SEQ ID NO:11and SEQ ID NO:31, SEQ ID NO:13 and SEQ ID NO:33, SEQ ID NO:15 and SEQ ID NO:35, SEQ ID NO:17 and SEQ ID NO:37, SEQ ID NO:19 and SEQ ID NO:39, or combinations of two or more of these primer sets. Furthermore, the composition may further comprise one or more probe, such as those set forth in Table 2 as SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, or SEQ ID NO:40.

A composition of the invention may comprise an amplification product of two primers. The amplification product may be provided as the major substance in the composition, as when provided in a purified or partially purified form, or may be present as a minority of the substances in the composition. As with each primer in a composition of the invention, the amplification product may be provided in any number of copies, in any amount, or at any concentration in the composition, advantageous amounts being easily identified by the practitioner for each particular purpose to which the amplification product will be applied. Non-limiting examples of compositions of the invention include compositions comprising an amplification product and one or more primers. Other non-limiting examples include compositions comprising an amplification product and a sample containing or suspected of containing a genome comprising a pre-selected unique sequence, an mRNA of interest, or both. Still other non-limiting examples of compositions comprise an amplification product and at least one amplification primer. Yet other non-limiting examples of compositions of the invention comprise an amplification product, at least one amplification primer, and at least one polymerase.

While it is envisioned that composition comprising a nucleic acid comprising a sequence present in a unique genomic sequence will often be one that comprises the nucleic acid as a result of amplification of the unique genomic sequence during an amplification reaction, it is to be noted that the composition may be made by providing a pre-formed nucleic acid comprising the unique genomic sequence. In such situations, the pre-formed nucleic acid will typically be provided as a control for one or more reactions, such as a positive control for the presence of the unique genomic sequence. However, it also may be added as a competitor for amplification of a bona fide unique genomic sequence from a cell's genome, or for any other reason chosen by the practitioner.

In embodiments, the composition comprises agarose, polyacrylamide, or some other polymeric material that is suitable for isolating or purifying, at least to some extent, nucleic acids, or for detecting nucleic acids. In embodiments, the composition comprises nylon, nitrocellulose, or some other solid support to which nucleic acids can bind. In some embodiments, the compositions comprise at least one label or member of a labeling system. Two or more different amplification products may be present in a single composition.

Compositions of the invention can comprise one or more nucleic acid polymerase. The polymerase can be any polymerase known to those of skill in the art as being useful for polymerizing a nucleic acid molecule from a primer using a strand of nucleic acid as a template for incorporation of nucleotide bases. Thus, it can be, for example, a reverse transcriptase, such as one isolated or derived from Maloney murine leukemia virus (MMLV) or the avian myoblastosis virus (AMV), Taq DNA polymerase, Pfu DNA polymerase, Pfx DNA polymerase, Tli DNA polymerase, Tfl DNA polymerase, klenow, T4 DNA polymerase, T3 RNA polymerase, T7 RNA polymerase, and SP6 RNA polymerase, or combinations of two or more thereof.

In exemplary embodiments, the compositions comprise some or all of the components necessary for amplification of nucleic acids, such as the components necessary for performing a PCR technique (e.g., RT-PCR or QRT-PCR). Thus, in embodiments, the compositions comprise Tris-HCl (e.g., about 50 mM, pH 8.3), KCl (e.g., about 75 mM), MgCl₂ (e.g., about 3 mM), dNTPs (e.g., about 800 ug each), oligo(dT18) or random primer (e.g., about 1 ug), polyadenylated RNA (e.g., about 5 ug), and reverse transcriptase, in a standard reaction volume, such as about 25 ul or about 50 ul. In other embodiments, the compositions comprise Tris-HCl (e.g., about 10 mM, pH 8.8), KCl (e.g., about 50 mM), MgCl₂ (e.g., about 1-5 mM) and one or more thermostable polymerases.

In a third aspect, the invention provides methods for quantitating the amount of sample provided in an amplification reaction. In general, the methods comprise providing a test sample containing genomic nucleic acid, amplifying one or more unique genomic sequences present in the genomic nucleic acid, comparing the amplification profile to a standard curve of amplification profiles obtained from reactions performed on reference samples from known numbers of original cells of the type from which the genomic nucleic acid originates, and determining the number of cells from which the test sample genomic nucleic acids originated. The method of the invention provides a control for QPCR reactions that can be used to correlate amplification profiles back to a known amount of starting material, such as a known amount of cells from which the nucleic acids being analyzed originate. The method, being based on quantitating a stable, known amount of a unique genomic sequence, a sequence that is intrinsic to the sample being tested, provides improved quantitation of QPCR reactions as compared to controls currently available in the art. It is also highly accurate, repeatable, and reproducible, which are features lacking in many of the currently available controls. Further, it is less complex to perform than many controls currently available in the art, particularly those that rely on addition of exogenous sequences as control sequences.

According to the method of the invention, providing can be any act that results in the stated substance, which in this case is the test sample, being present in a particular environment. Broadly speaking in regard to providing a test sample, it can be any action that results in the practitioner obtaining and having in possession the test sample of interest in a form suitable for use in the present method (the term “assay” being used herein interchangeable on occasion). Those of skill in the art are aware of numerous actions that can achieve this result. In addition, non-limiting examples are provided throughout this disclosure. For example, providing can be obtaining cells and lysing them to create a cell lysate. Obtaining the cells can be through any number of activities, including, but not limited to, 1) growing or culturing cells in laboratory media or a host organism, 2) removing cells from a multi-cellular organism (e.g., removing blood cells, liver cells, neuronal cells, etc.), 3) receiving cells from another who has grown or cultured the cells, or removed the cells from a multi-cellular organism, 4) receiving fresh cells from any source, and 5) receiving frozen cells from any source.

The act of providing preferably includes lysis of the cells to generate a cell lysate. Numerous techniques for lysis of eukaryotic and prokaryotic cells are known in the art, and any suitable technique may be used. A particularly useful technique for lysis of eukaryotic cells involves the use of a one-step lysis and amplification buffer, such as those commercially available from various vendors, and that disclosed in U.S. patent application Ser. No. 11/152,773.

In a more general sense than specifically relating to providing a test sample, providing can include mixing two or more substances together to create a composition or mixture. It can also include isolating a substance or composition from its natural environment or the environment from which it came. Providing likewise can include obtaining a substance or composition in a purified or partially purified form from a supplier or vendor. Additionally, providing can include obtaining a sample suspected of containing an mRNA of interest, removing a portion for use in the present method, and maintaining the remaining amount of sample in a separate container from the portion to be used in the present method. Some or all of the remaining portion of the sample can be used for any number of purposes, including, but not limited to, performing identical reactions to confirm the reproducibility of the method for that particular sample, performing an amplification reaction on a target nucleic acid, such as a nucleic acid that is suspected of being expressed in the cell from which the test sample originated.

In the present method, a test sample is any substance that comprises, or is suspected of comprising, genomic nucleic acid of a cell of interest. The genomic nucleic acid is typically cellular DNA, but may be viral DNA or RNA as well. The genomic nucleic acid may be a single molecule, such as is a single chromosome of a prokaryote, or may be multiple molecules, such as multiple chromosomes (e.g., two or more copies of the same chromosome or one or more copies of two or more different chromosomes) of in a eukaryotic organism or one or more chromosomes and one or more stable extrachromosomal molecules in a prokaryote or eukaryote. Thus, the test sample may be one that comprises cells, cell lysates, or combinations of the two. Likewise, the test sample may comprise isolated or purified (to any extent) nucleic acids from a cell or virus, or mixture of cell and virus.

The test sample may originate from any organism. It often originates from cells and tissues from mammals, including, but not limited to, humans. Any mammalian cell or tissue is suitable for use as a source for the test sample. Furthermore, any human cell or tissue is suitable for use as a source for the test sample. Included among these cells are blood cells, neuronal cells, muscle cells, heart cells, kidney cells, liver cells, bone cells, and any other cell from an organ or tissue. Further, cells may be of any type, including, but not limited to, fibroblasts, neuroblasts, leukocytes, and the like. In addition, cells may be from any differentiation state of an organism or from any stage in a disease or disorder. Thus, cells can be embryonic cells, adult cells, cancer cells (or otherwise neoplastic cells), primary cells or established cell lines (for example, human hepatocytes, human keratinocytes, human umbilical vein endothelial cells, human microvascular endothelial cells, human fibroblasts, rat hepatocytes, mouse hepatocytes, rat liver stellate cells, Kupffer cells, human aortic smooth muscle cells, human bronchial epithelial cells, B lymphoma cell lines, A549, BHK, C2C12 myoblasts, CHO, Cos-1, Cos-7, HEK 293, HeLa, HepG2, Jurkat, HT-29, MCF-7, NIH 3T3, NDCK, PC-12, K-562), and other cells of medical or scientific interest.

According to the method of the invention, the act of amplifying includes any in vitro technique that results in an increase in the number of copies of one or more pre-selected nucleic acid sequences in a composition comprising those unique genomic sequences. In embodiments, the technique further comprises increasing the number of copies of at least one pre-selected target nucleic acid of interest, which is typically, but not necessarily, an mRNA (or its corresponding cDNA) of interest. When the method comprises amplifying both unique genomic sequence(s) and target sequence(s) of interest, the amplifying of each can be performed in a reaction mixture that is separate from all others or in a reaction mixture that is in common with one, some, or all of the other amplifying reactions.

Currently, the most common and powerful in vitro technique for amplifying nucleic acids of interest is PCR. Variations of the original PCR technique have been developed to take advantage of the different characteristics of target nucleic acids, including whether the nucleic acid is DNA or RNA, the concentration of the nucleic acid in the sample, the need to monitor the amplification in real-time, etc. Any suitable PCR technique practiced in the art is envisioned as a way to accomplish amplifying of unique genomic sequences, target sequences, or both. In particular, QPCR techniques, including QRT-PCR, are envisioned as techniques for amplifying according to the present method.

In embodiments, the method of the invention comprises comparing the results of amplification of one or more unique genomic sequences to a standard curve. Accordingly, the present invention, in embodiments, comprises creation of a standard curve. The standard curve can be created by amplifying known amounts of starting materials (e.g., a unique genomic sequence from lysates of a known number of cells), and plotting the results of the amplification reactions on a graph. It is preferred that the standard curve be made by amplifying a unique genomic sequence from multiple samples, each originating from a different number of cells. Those of skill in the art are well aware of techniques for making standard curves, including those for quantitation of QPCR reactions, and any suitable technique may be used to create the standard curve for use in the present methods.

The standard curve is preferably created from cellular material that is of the same cell type or organism from which the test sample originates. For example, the standard curve can be created from a portion of a sample from which the test sample originates. Likewise, the standard curve can be created from cultures of the same cell that were obtained at a time earlier than the cells from which the test sample is derived. It is preferred that the standard curve be created from cells of the same cell type (where the cells are from a multicellular organism) or the same species or strain (where the cells are from a unicellular organism). However, because organisms have extremely high levels of genomic identity among tissue types, cell types, and strains, the standard curve may be created from any cells from the same species that serves as the source of the test sample. Thus, any human cell, regardless of cell or tissue type, may be used as a source for a standard curve for any test sample originating from a human cell. Of course, if the source cell for the standard curve or the test sample is known to have a genetic duplication that affects the copy number of the unique genomic sequence in either sample, appropriate correction should be made. Further, if either has a genetic deletion that affects the copy number of either sample, a new cell or a different primer or primer set should be used for the standard curve or test sample. Variation in sequence copy number may be an important consideration in creating standard curves and assaying test samples from cells from tumors, cancers, neoplasias, etc., or that relate to genetic disorders involving chromosomal rearrangements, duplications, deletions, and the like. The number of cells used for creating the standard curve may be determined by any suitable method, such as FACS counting, light scattering (e.g., OD readings), and the like. Such techniques are well known to those of skill in the art, and need not be detailed here.

In other embodiments, a standard curve is not necessary. For example, in some embodiments, the method is a method of semi-quantitative or qualitative determination of a nucleic acid of interest in a sample. In practice of the method of the invention in those embodiments, the genomic DNA, and in particular the unique sequence, can be used as a normalizer for comparison of gene expression between samples. In cases where two samples have unknown amounts of cells, and thus nucleic acids, amplification of a unique sequence in common between the two samples can allow a person to determine the relative amounts of nucleic acid levels of interest (e.g., a particular mRNA species) by comparing the amplification profile of the unique sequence between the two samples, and normalizing each to the other. Of course, this procedure may be used to normalize multiple samples beyond just two. The normalization may be preformed as the sole basis for normalization, or may be performed in conjunction with other normalization techniques, such as comparison of expression of “housekeeping genes”.

As is apparent by the description of the invention above, in embodiments, the methods further comprise performing an amplification reaction with primers that are specific for a nucleic acid sequence of interest (a target expression sequence). That is, the method can further comprise amplifying one or more target expression sequence. When the methods comprise amplification of a target expression sequence, the two amplification reactions (unique genomic sequence and target expression sequence) can be performed in the same reaction mixture or in two or more separate reaction mixtures. When performed in separate reaction mixtures, it is preferred that the reaction mixtures be as similar to each other as possible, in both composition and treatment. For example, it is preferred that the only difference between the reaction mixtures be the presence or absence of the primers that are specific for either the unique genomic sequence(s) or the target expression sequence(s), and that the two reaction mixtures be treated identically.

According to the present methods, when target expression sequences are amplified, their amplification profiles can be accurately compared to amplification profiles of similar or different target expression sequences from the same or other samples by normalizing the amount of starting target expression sequences between samples. This can be accomplished by normalizing the number of original cells from which each sample was obtained. Using unique genomic nucleic acid sequences to normalize different samples provides a powerful, accurate method for comparing the expression levels of a target sequence between samples of different cell types, and between samples of the same cell type taken at different times or from different sources (e.g., a patient with cancer and a healthy patient). It avoids problems associated with variable expression of housekeeping genes between cell types and development stages, differences in amplification efficiencies between different nucleic acid sequences, and other shortcomings of the methods currently practiced in the art.

In another aspect, the invention provides kits. In general, the kits contain some or all of the components necessary to practice an embodiment of the method of the invention. Thus, for example, the kits may contain one or more primer or one or more composition of the invention. Likewise, the kits may contain multiple primers, or sets of primers, for amplification of unique genomic sequences or for amplification of target expression sequences. In typical embodiments, a kit comprises at least one container that contains a nucleic acid of the invention. In various specific embodiments, the kit comprises all of the nucleic acids needed to perform at least one embodiment of the method of the invention.

Kits are generally defined as packages containing one or more containers containing one or more nucleic acids or compositions of the invention or one or more substances useful for practicing at least one embodiment of the method of the invention. The kits themselves may be fabricated out of any suitable material, including, but not limited to, cardboard, metal, glass, plastic, or some other polymeric material known to be useful for packaging and storing biological samples, research reagents, or substances. The kits may be designed to hold one or more containers, each of such containers being designed to hold one or more nucleic acids, compositions, or samples of the invention. The containers may be fabricated out of any suitable material including, but not limited to, glass, metal, plastic, or some other suitable polymeric material. Each container may be selected independently for material, shape, and size. Non-limiting examples of containers include tubes (e.g., microfuge tubes), vials, ampules, bottles, jars, bags, and the like. Each container may be sealed with a permanent seal or a recloseable seal, such as a flip-top or a screw cap. One or more of the containers in the kit may be sterilized prior to or after inclusion in the kit.

The kit of the invention may include one or more other components or substances useful in practicing the methods of the invention, such as sterile water or aqueous solutions, buffers for performing the various reactions involved in the methods of the invention, and/or reagents for detection of amplification products. Thus, in embodiments, the kit comprises one or more polymerase for amplification of a unique genomic sequence, a target expression sequence, or both. It also can comprise some or all of the components, reagents, and supplies for performing amplification according to embodiments of the invention. In embodiments, it includes some or all of the reagents necessary for performing a QPCR technique, including, but not limited to QRT-PCR.

In certain embodiments, the kits comprise printed materials describing practice of a method of the invention. In embodiments, the kits comprise printed standard curves for one or more organisms (e.g., human), cell types (e.g., leukocytes), or specific cells (e.g., HeLa cells).

For example, a kit according to the invention may comprise a container containing one or more primer for amplification of a unique genomic sequence of a cell of interest. In embodiments, the primer may be one or more primer comprising a sequence defined by SEQ ID NO:1 through SEQ ID NO:20 or any other sequence disclosed herein. The primers, if more than one primer is provided in a particular kit, may be provided in separate containers (e.g., one or more primer in each container) or all in a single container. Furthermore, a single kit may comprise multiple containers, each independently containing one or more primer of the invention. In preferred embodiments, each container contains a sufficient number of primers to amplify at least one unique genomic sequence of interest (if present) in a sample. Thus, in embodiments, one or more container in the kit comprises two primers, both specific for a particular unique genomic sequence. The amounts of each primer provided in each kit may be any amounts suitable or convenient for practice of at least one embodiment of the method of the invention. Thus, each primer may be independently provided in amounts from the picogram range to milligram or greater range. Typically, primers will be provided in the kit in microgram ranges for dilution before use in a method of the invention, or in nanogram to picogram amount for direct use in a method of the invention.

A kit of the invention can comprise at least one primer for amplification of a nucleic acid of interest, such as a target expression sequence of interest. In such kits, the primer(s) are provided in a similar manner as primer(s) for amplification of unique genomic sequences. That is, they may be provided in any permutation of identities and amounts per container, and in any number of containers per kit.

Kits of the invention may also comprise one or more probes for detection of amplification of one or more unique genomic sequences or one or more target sequences (e.g., mRNA species). The specificity of probes to be included in kits are preferably provided in conjunction and in consideration of the primer(s) provided in the kit for amplification of the unique genomic sequence(s) and/or in conjunction and consideration of the primer(s) provided in the kit for amplification of a target sequence of interest.

In certain embodiments of the kits, one or more polymerase is provided. Typically, the polymerase is one that is capable of copying a nucleic acid template in a PCR reaction. Non-limiting, exemplary suitable polymerases are mentioned above, and include both reverse transcriptases and thermostable DNA polymerases. In the kits, each polymerase may be provided in its own container, or multiple polymerases may be provided in a single container, which can comprise other components that are useful for practicing one or more embodiment of the method of the invention (e.g., salts, buffers).

Certain arrangements of the kit of the invention comprise some or all of the reagents necessary for performing a QPCR reaction. In embodiments, some or all of the reagents necessary for performing a QRT-PCR reaction are included in the kit. Any suitable configuration of components in containers is envisioned by the invention, the practitioner being capable of determining the most desirable configuration for each particular use of the kit.

In exemplary embodiments of the kit, the kit comprises a buffer that is suitable for use in both lysing cells and amplifying nucleic acids liberated from the cells through such lysis. Various “one-step” lysis and amplification buffers are known in the art, and any of these may be included in a kit of the invention. In addition, a novel one-step lysis and amplification buffer is disclosed in U.S. patent application Ser. No. 11/152/773. This buffer may advantageously be provided in the kit of the invention in one or more containers. Any suitable volume of buffer may be provided in each container.

Non-limiting examples of kits in which the primers and/or probes of the present invention may be included are: an mRNA isolation kit, such as that sold by Stratagene as the Absolutely mRNA™ Isolation Kit (Catalog Number 400806); a QPCR cDNA synthesis kit, such as that sold by Stratagene as the StrataScript QPCR cDNA Synthesis Kit (Catalog Number 600554); a kit comprising QPCR reagents and/or buffers, such as that sold by Stratagene as the Brilliant® SYBR® Green QPCR Master Mix Kit (Catalog Number 600548), a kit comprising QRT-PCR reagents and/or buffers for 1-step QRT-PCR, such as that sold by Stratagene as the Brilliant® SYBR® Green QRT-PCR Master Mix Kit (Catalog Number 600552), and a kit comprising QRT-PCR reagents and/or buffers for 2-step QRT-PCR, such as that sold by Stratagene as the Brilliant® SYBR® Green QRT-PCR Master Mix Kit.

### EXAMPLES

The invention will be further explained by the following Examples, which are intended to be purely exemplary of the invention, and should not be considered as limiting the invention in any way.

### Example 1

### Identification of Unique Human Genomic Sequences

To provide internal controls for amplification of target nucleic acids, and for normalization of materials to be amplified across samples, unique sequences within the human genome were identified, and primer sets designed to amplify those unique sequences.

Human genome, UCSC Build hg17, was used as a source of human unique genomic sequences. Fifteen 600-nucleotide repeat-free fragments were selected on each chromosome, except chromosomes X and Y. Each of the 315 selected fragments were aligned to the entire human genome using blastn (Altschul et al., 1990). The alignment analysis revealed 237 sequences that matched single genomic regions with e-values of 1e-10 or lower. These 237 sequences were used for primer and probe design for amplification of unique human genomic sequences.

60° C. and 70° C. temperatures were requested as primer and probe melting temperatures, respectively. Additionally, the probe design had the following parameters: 1) the “C” content must be higher than the “G” content; 2) the 5′-end of the probe must not be “G”; and 3) the distance between the 3′-end of the upstream primer and the 5′-end of the probe must not exceed 8 nucleotides. Probes were used in conjunction with primers for assays involving TaqMan® assays.

Primers and probes were designed using the primer3-1.0.0 program (Rozen and Skaletsky, 2000). Two sets of primers or primer/probe combinations were designed. In a first round, one hundred six combinations of two primers and one probe were created. Because the length of amplicon preferred for SYBR® Green dye detection of amplification products is considerably longer than that for TaqMan® (e.g., about 200 bp for SYBR® Green dye compared to about 80-100 bp for TaqMan®), in a second round of design, new downstream primers were created for use in QPCR reactions using SYBR® Green dye (i.e., without a probe). The same upstream primers were used for both types of detection assays. In the second round of design, 60 sets of primers were designed. Ten primer sets for ten different human chromosomes are provided in Table 1, above. Table 2, above, provides exemplary corresponding probes.

### Example 2

### Generation of Amplification Plots, Dissociation Curves, and a Standard Curve for a Human Cell Line

Amplification profiles of unique genomic sequences in the genome of the human HeLa cell line were generated using primer pairs disclosed in Table 1, above. In addition, a standard curve plotting the amplification profile of the unique human genomic sequence detected by primer set 10 (SEQ ID NO:19 and SEQ ID NO:20) versus amount of genomic DNA present in the sample was created. The QPCR reactions were performed and monitored using an MX3000P thermocycler from Stratagene, Brilliant® SYBR® Green QPCR Master Mix (Stratagene Cat. No. 600548) and HeLa genomic DNA (10 ng, 1 ng, 0.1 ng, and 0.01 ng per amplification reaction), according to the instructions provided with the Brilliant® SYBR® Green QPCR Master Mix. Briefly, the amplification reactions comprised the following components and amounts (total volume of 25 ul): 5 ul of gDNA; 12.5 ul of 2×Master Mix; 0.125 ul of each primer (15 uM); and 0.375 ul of diluted (1:500) ROX reference dye. PCR amplifications were performed as follows: 1 cycle of 10 minutes at 95° C.; 40 cycles comprising 30 seconds at 95° C., 1 minute at 60° C., and 1 minute at 72° C.

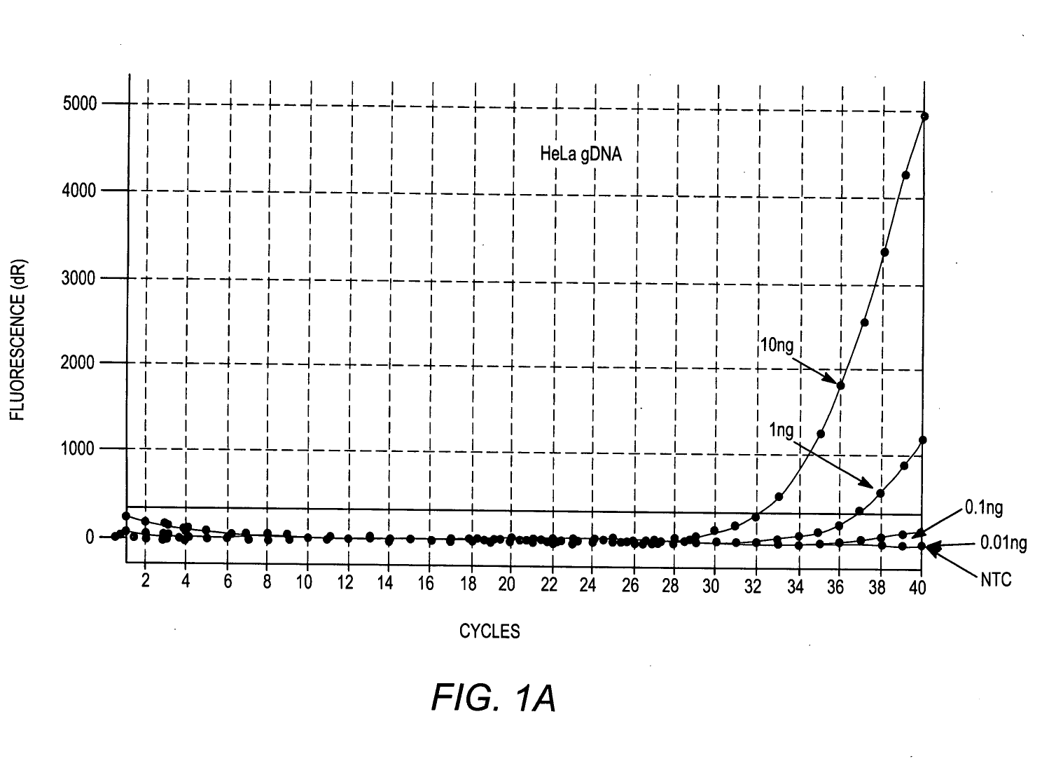

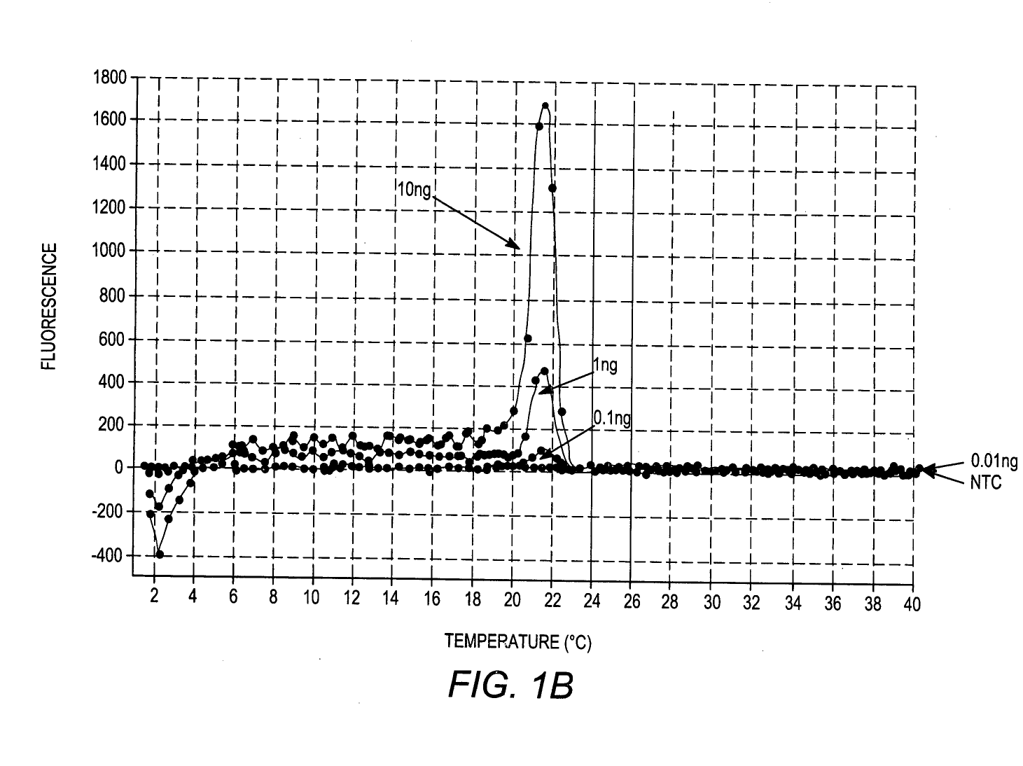

Following amplification, dissociation profiles of the amplification products were generated to confirm the purity of the amplification products and provide an indication of the identity of the amplification products. Dissociation curves were generated as follows: Prior to generating the dissociation curves, the amplification reactions were incubated for 1 minute at 95° C. to denature the PCR products. The temperature was then ramped down to 55° C. For the dissociation curve, the temperature was then ramped up from 55° C. to 95° C. at 0.2° C. per second. Fluorescence data was continuously collected during the 55°-95° C. ramp up. The dissociation curves are presented in FIGS. 1B, 2B, 3B, 4B, and 5B.

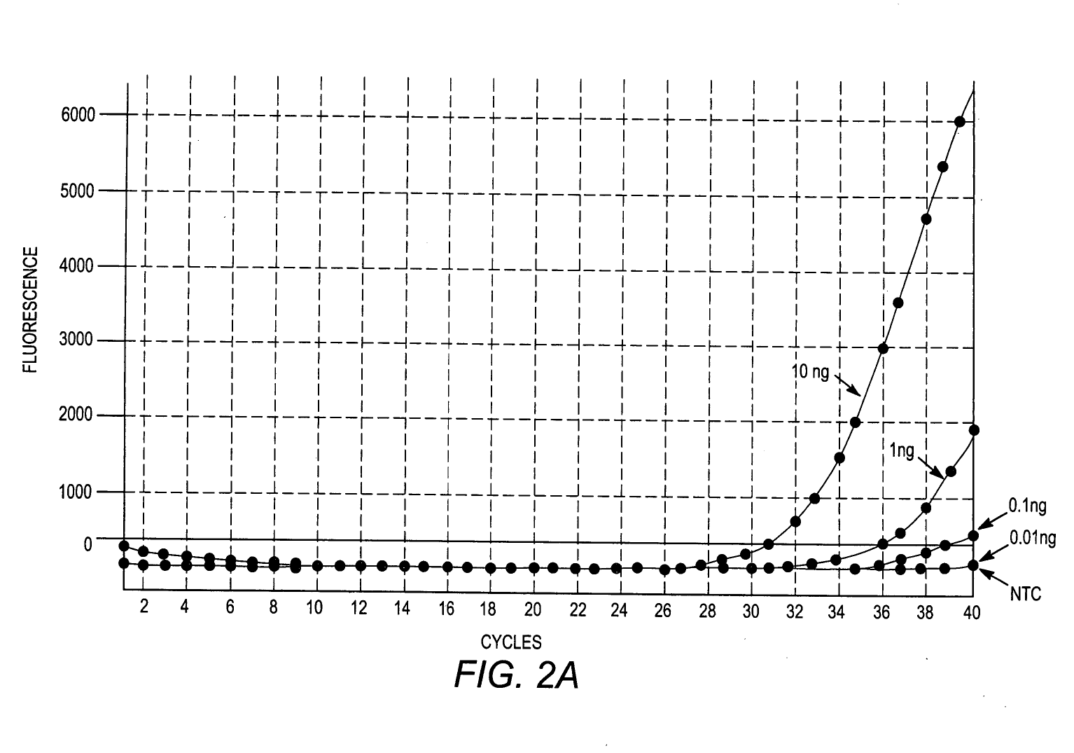

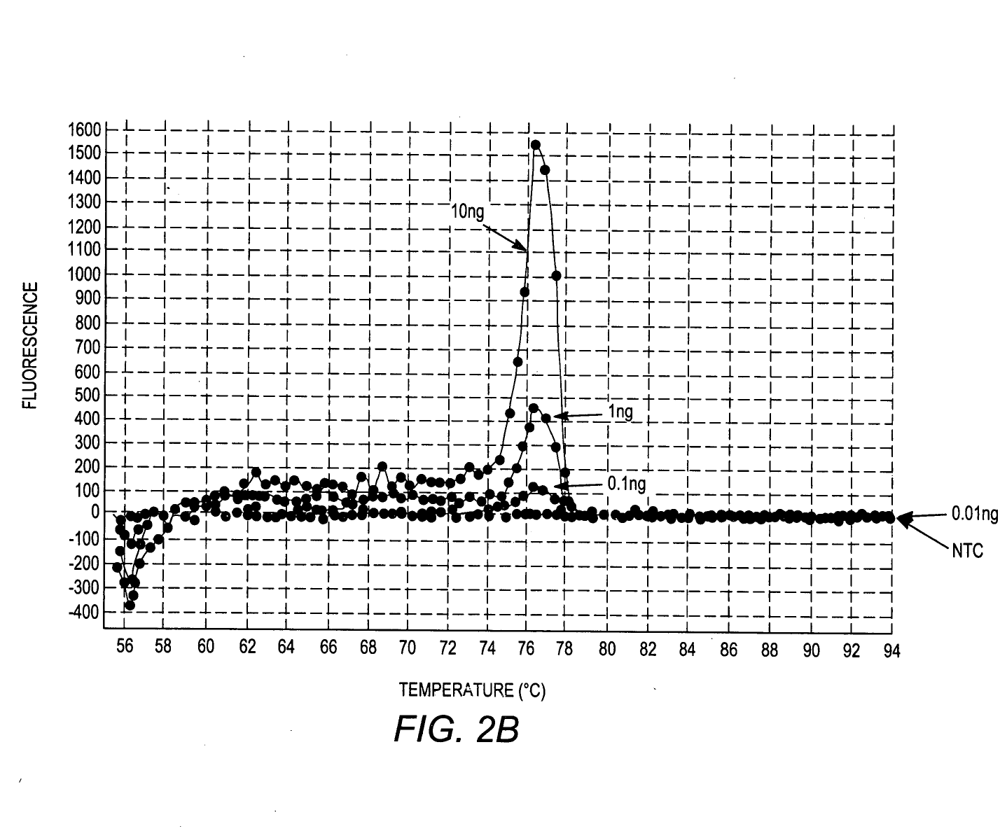

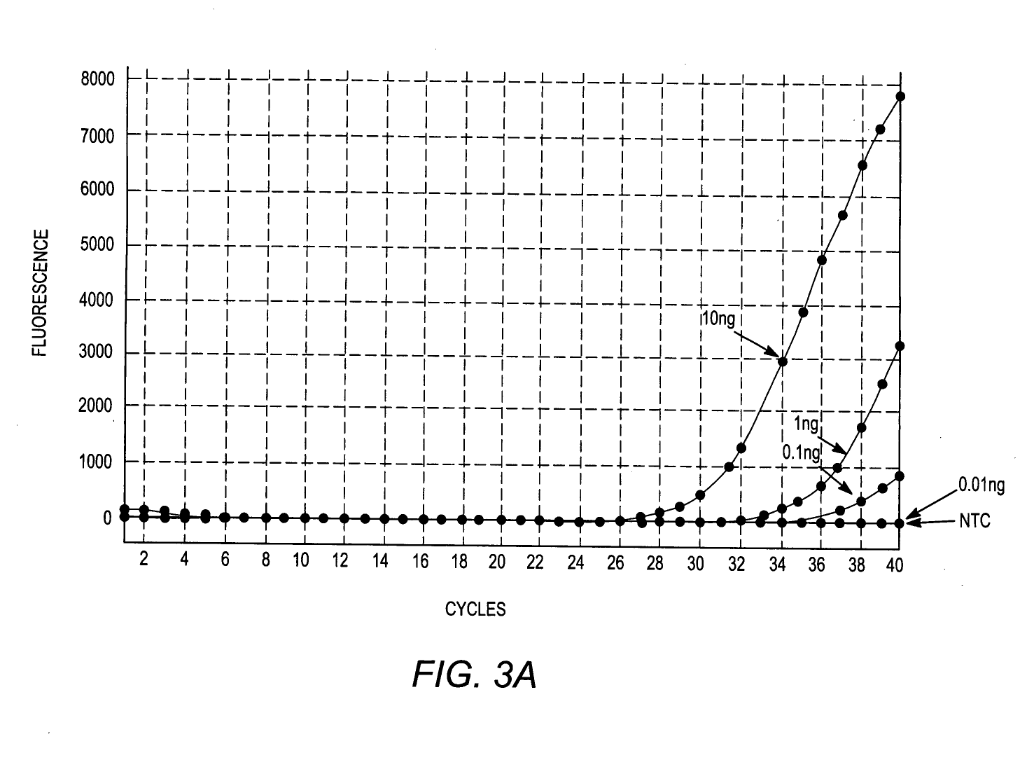

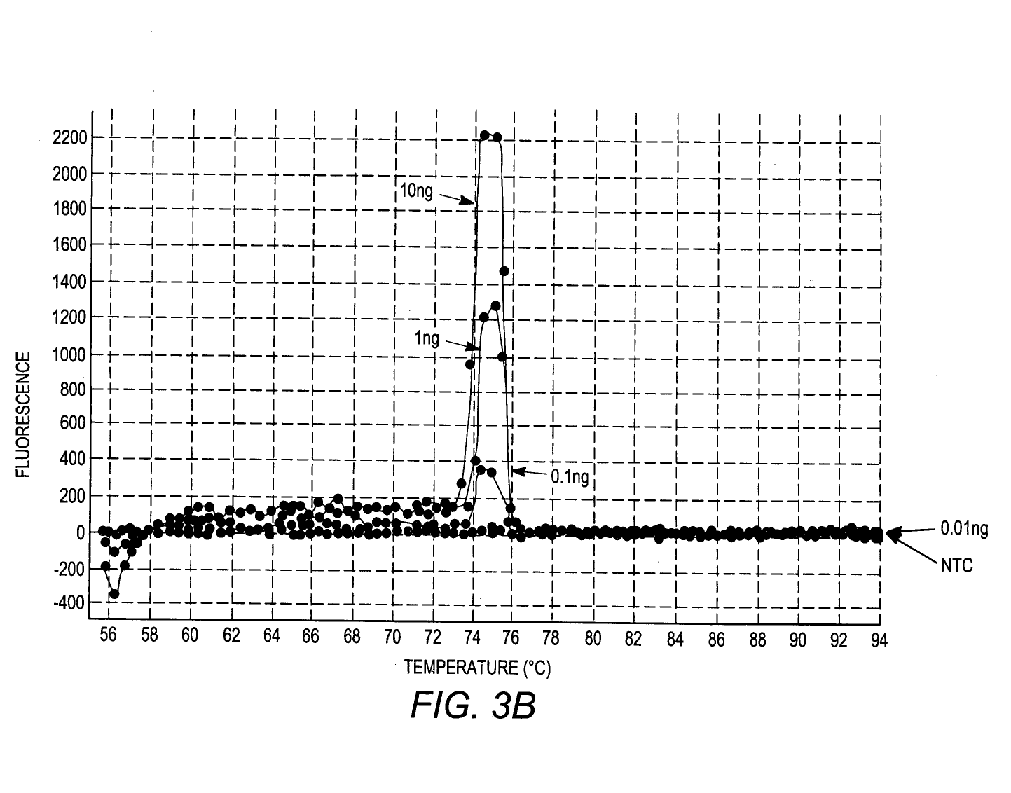

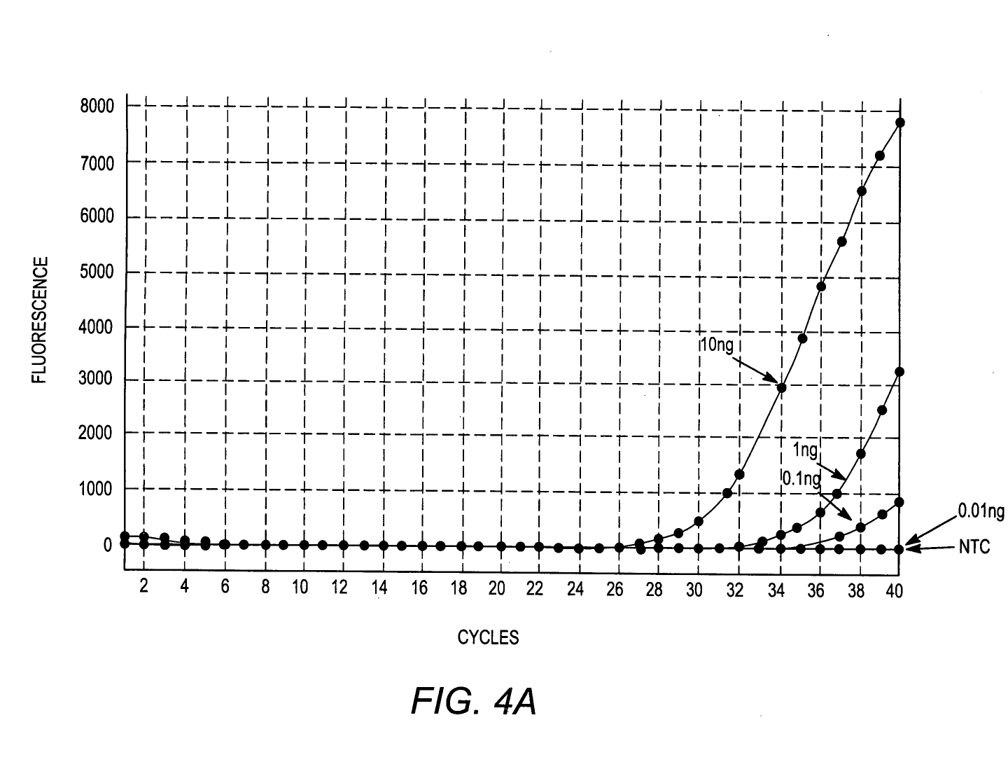

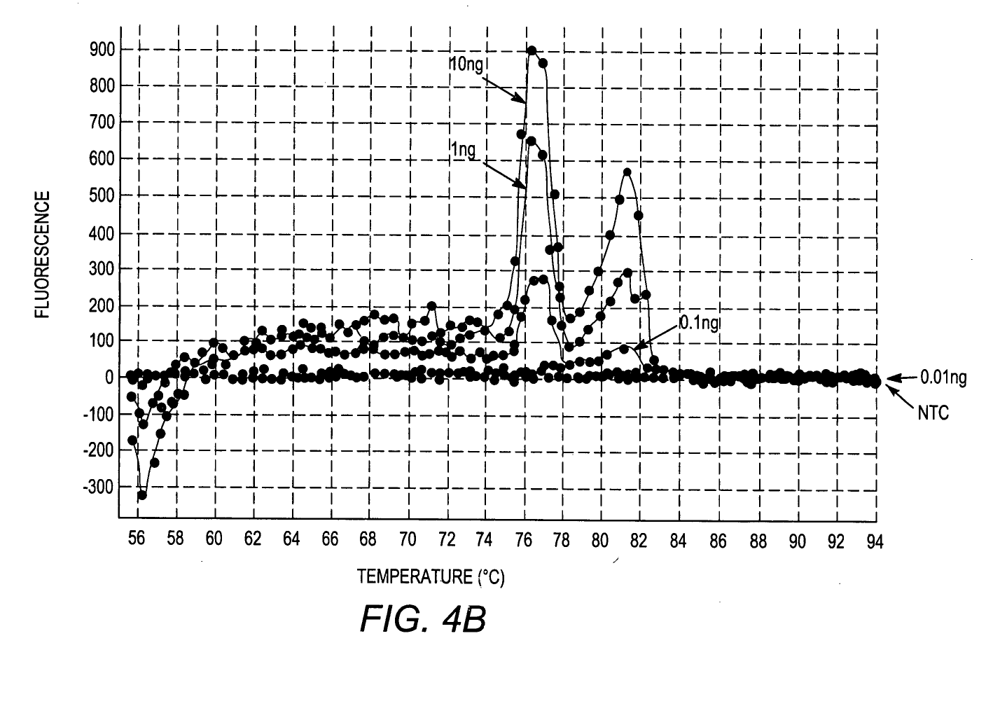

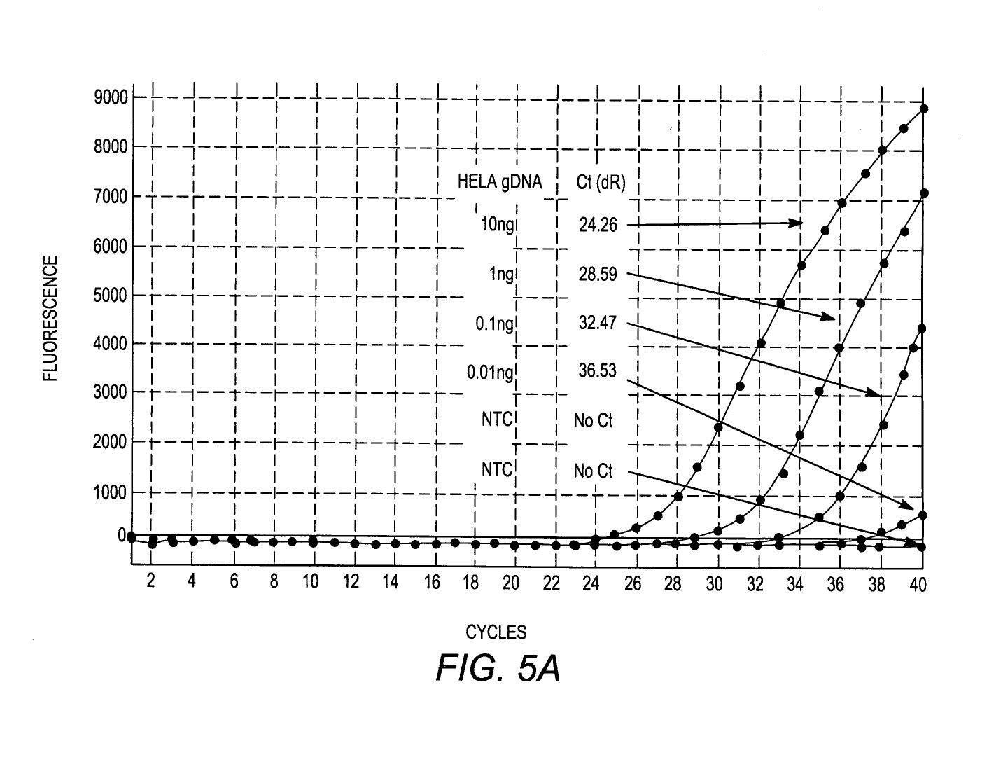

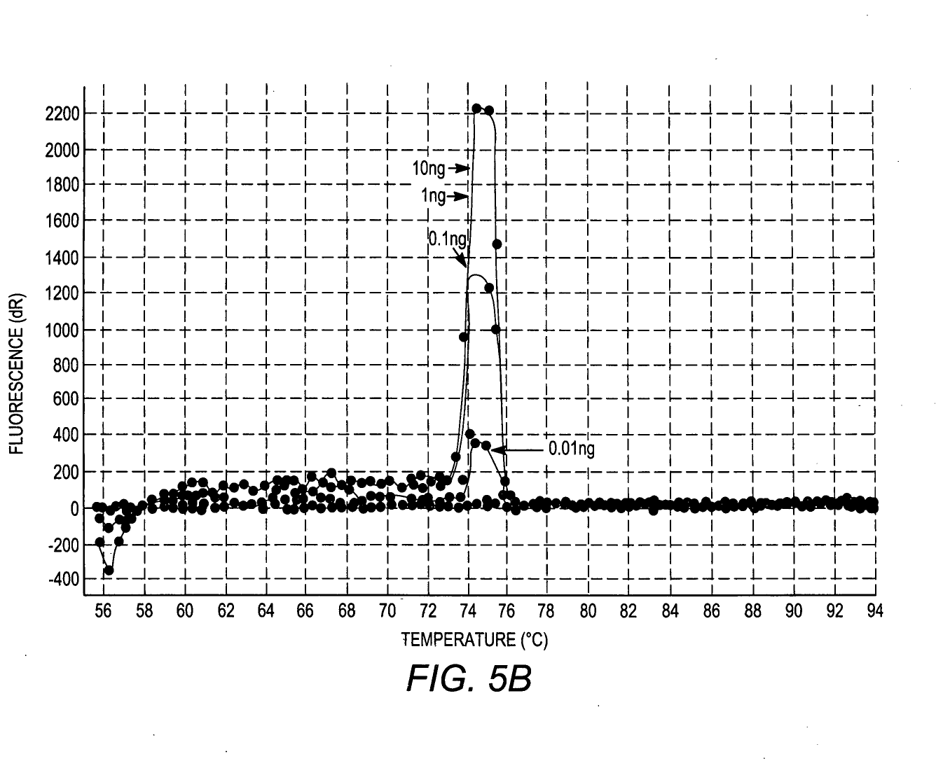

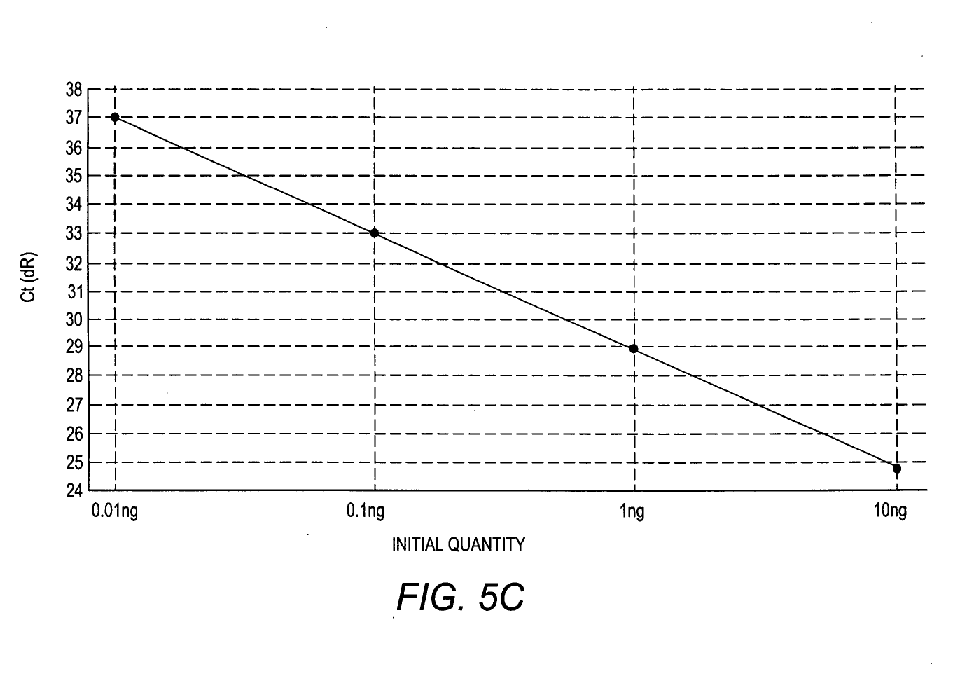

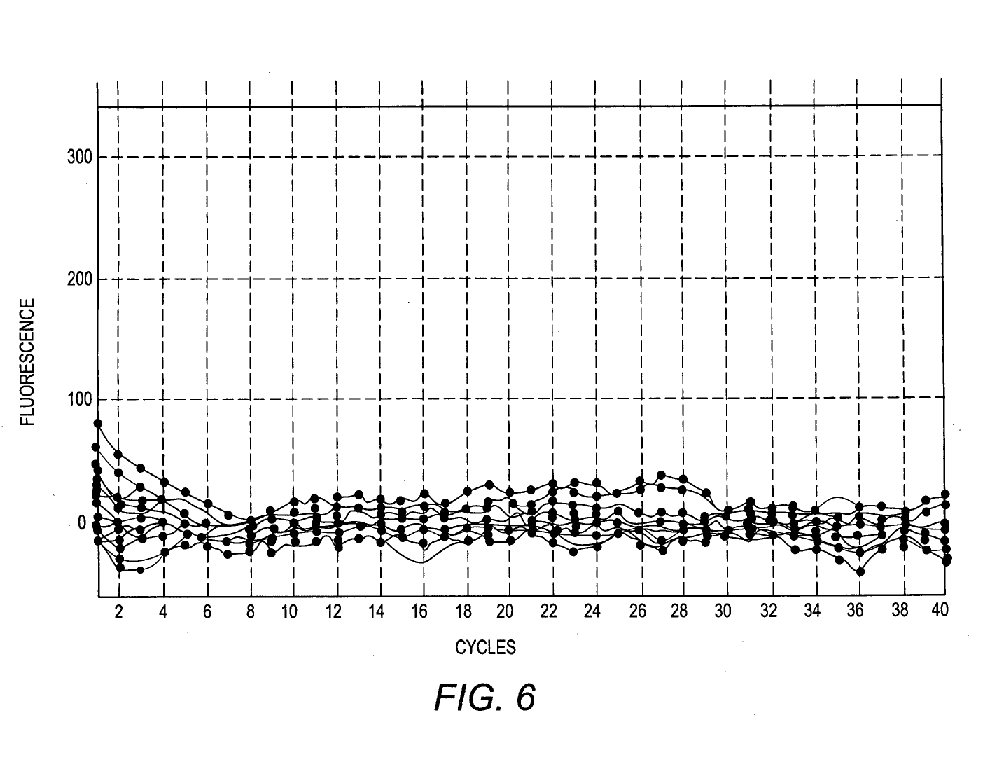

The amplification plots and corresponding dissociation curves for selected primer sets are presented in FIGS. 1A through 5B. In addition, a standard curve for amplification of HeLa gDNA with primer set 10 (SEQ ID NO:19 and SEQ ID NO:20), which plots Ct vs. initial quantity of HeLa gDNA in the reaction, is presented in FIG. 5C. A “no template” control amplification plot, depicting amplification using the ten primer sets presented in Table 1, above, is presented in FIG. 6. More specifically, FIGS. 1A and 1B show amplification of HeLa gDNA using primer set 2 (SEQ ID NO:3 and SEQ ID NO:4), and dissociation of the amplification product. FIGS. 2A and 2B show amplification of HeLa gDNA using primer set 3 (SEQ ID NO:5 and SEQ ID NO:6), and dissociation of the amplification product. FIGS. 3A and 3B show amplification of HeLa gDNA using primer set 8 (SEQ ID NO:15 and SEQ ID NO:16), and dissociation of the amplification product. FIGS. 4A and 4B show amplification of HeLa gDNA using primer set 9 (SEQ ID NO:17 and SEQ ID NO:18), and dissociation of the amplification product. FIGS. 5A and 5B show amplification of HeLa gDNA using primer set 10 (SEQ ID NO:19 and SEQ ID NO:20), and dissociation of the amplification product. FIG. 5C depicts a standard curve produced from the data presented in FIG. 5A, plotting Ct values vs. amount of HeLa gDNA in the amplification reaction.

As can be seen from the amplification plots, primer sets that are specific for unique sequences in genomic HeLa DNA can be used to amplify the HeLa genomic DNA from as little as 0.01 ng of gDNA or less. The corresponding dissociation plots indicate that amplification for each primer set was specific for a single nucleotide sequence on the gDNA. Furthermore, the standard curve presented in FIG. 5C shows that the amplification reactions, as evaluated by Ct values, are linear across at least 3 orders of magnitude, from as little or less than 0.01 ng up to 10 ng or more.

FIG. 6 shows that primer-dimer formation and amplification is not observed with any of the ten primer sets disclosed in Table 1, above. More specifically, PCR reactions using the same reaction conditions disclosed above, but without the target template, were run to determine if detected amplification products were due to specific amplification of target sequences or due to amplification of primer dimers. The results show that no amplification occurred, indicating that none of the tested ten primer sets amplify primer dimers. This is consistent with the dissociation curves presented in FIGS. 1B, 2B, 3B, 4B, and 5B.

In general, FIGS. 1A through 6 show that gDNA can be used as a control for QPCR reactions. Furthermore, the gDNA yields a robust standard curve for amplification vs. amount of starting material. Because the number of copies of the gDNA sequences being amplified is known per cell, the amplification profiles and standard curve can be used to normalize samples containing unknown amounts of human cells. Furthermore, the data show that, although all primer sets work to some extent with one or more cell type, primer sets 2 and 10 work well across multiple cell types.

### Example 3

### Evaluation of Primer Performance With Different gDNA Samples

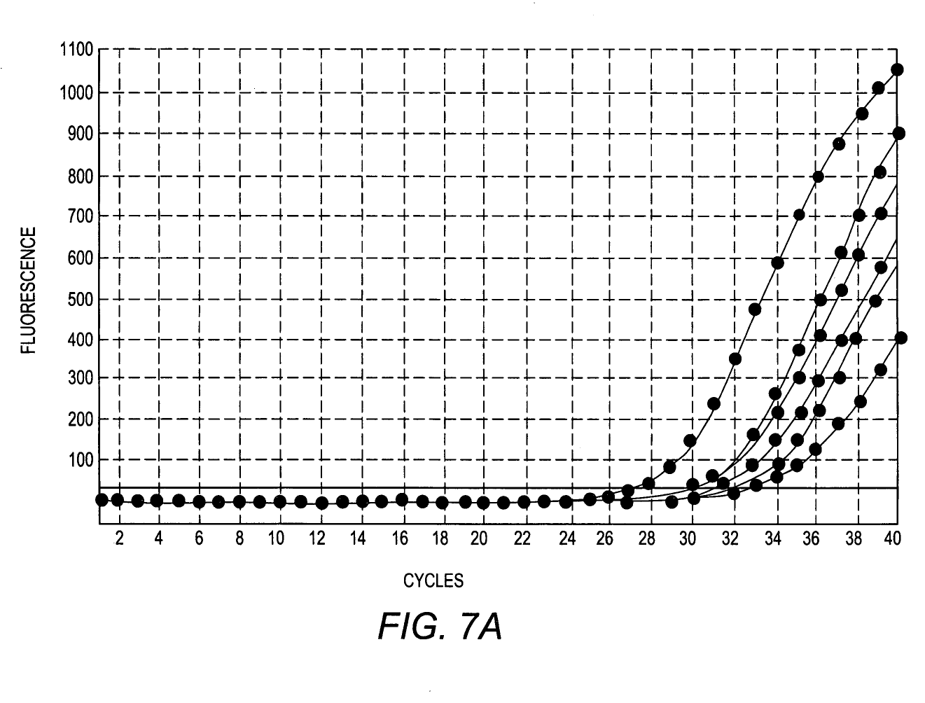

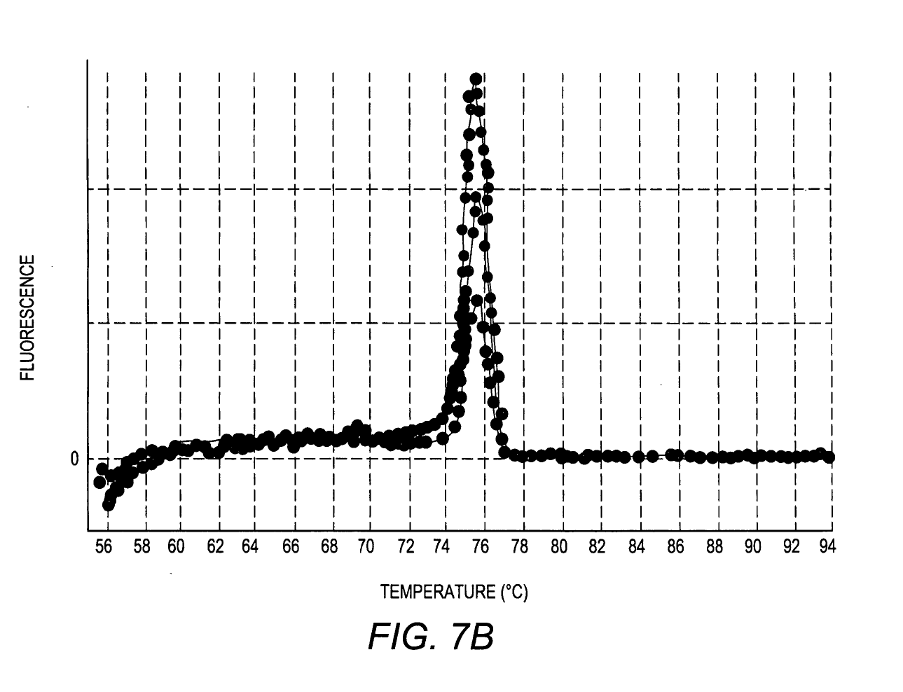

The ten primer sets from Table 1 were used to determine whether the primer sets were suitable for amplification of unique gDNA sequences from different human cell types. To do so, eight human cell types (A2058, SW872, HepG2, U937, RPMI8226, NTERA, human gDNA from Clontech, and human gDNA from Promega) were selected and the ten primer sets were used in ten separate amplification reactions. Ten corresponding dissociation curves were generated from the ten amplification reactions. Amplification reactions were performed, and dissociation curves were obtained, using the reagents, procedures, and equipment outlined above, with the exception that, for each amplification reaction, 1 ng of gDNA was provided. The amplification plots and dissociation curves are depicted in FIGS. 7A through 16B.

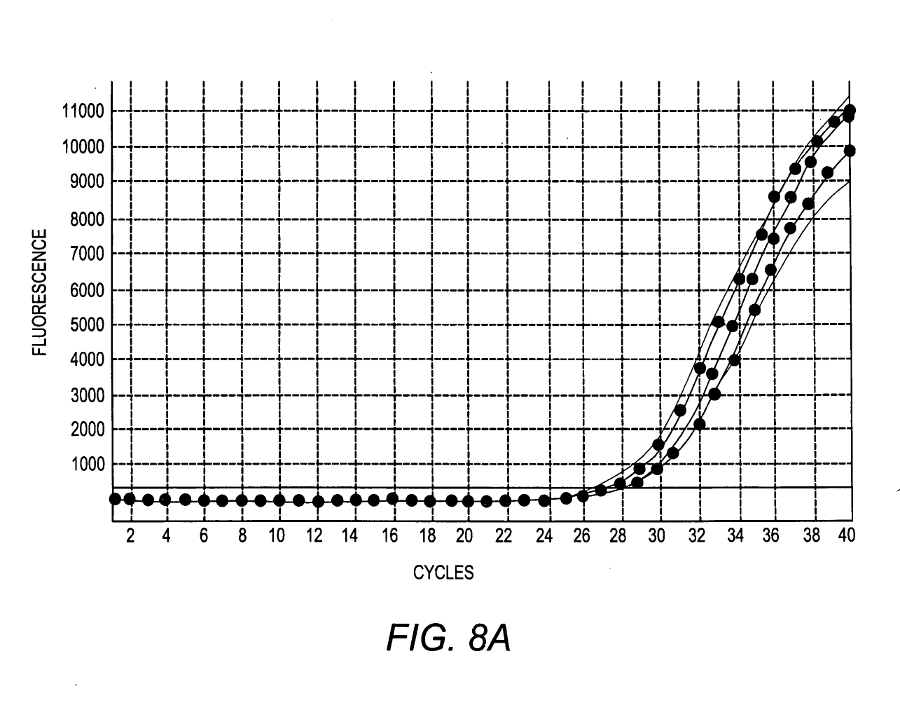

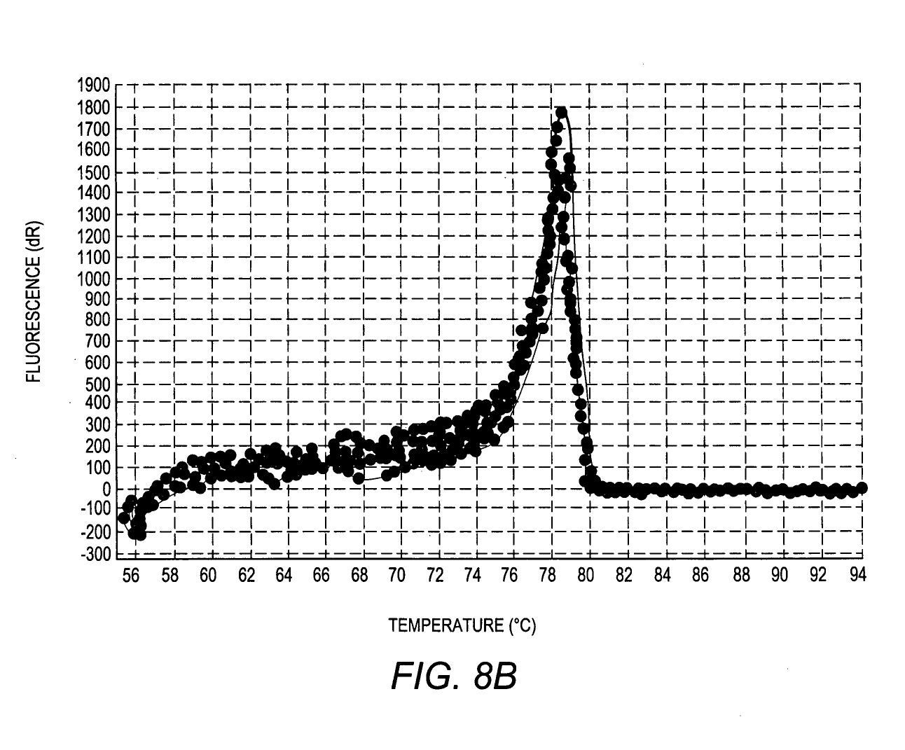

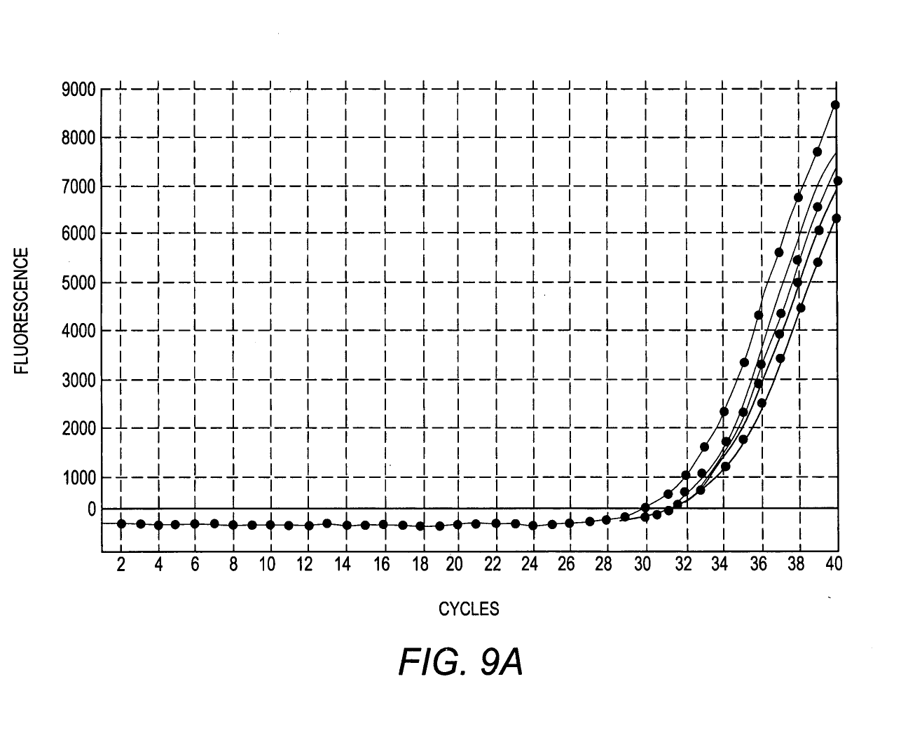

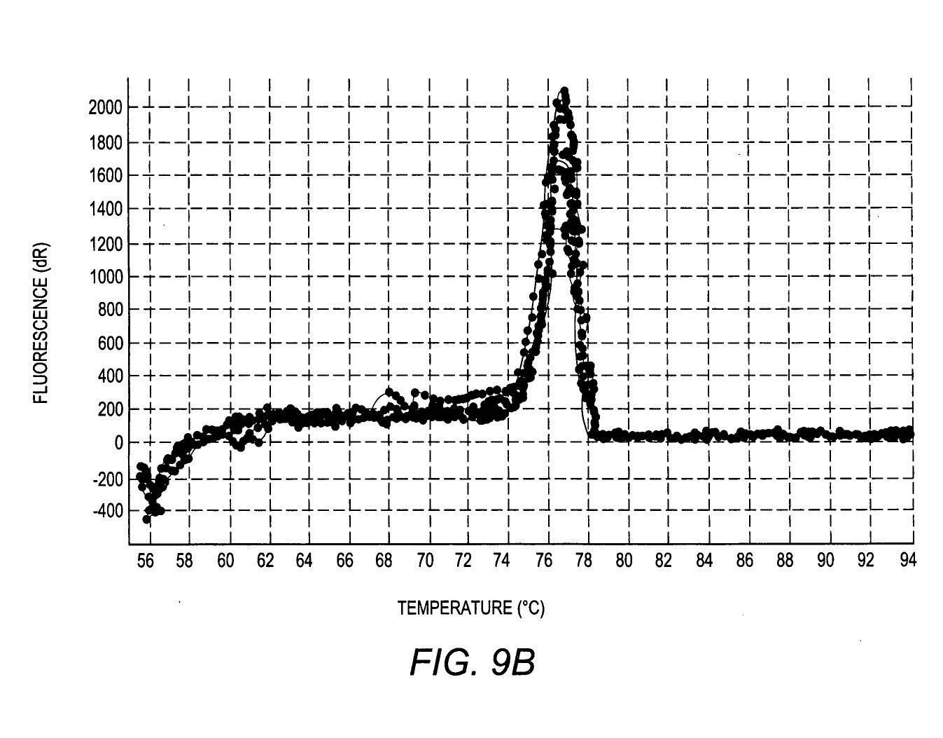

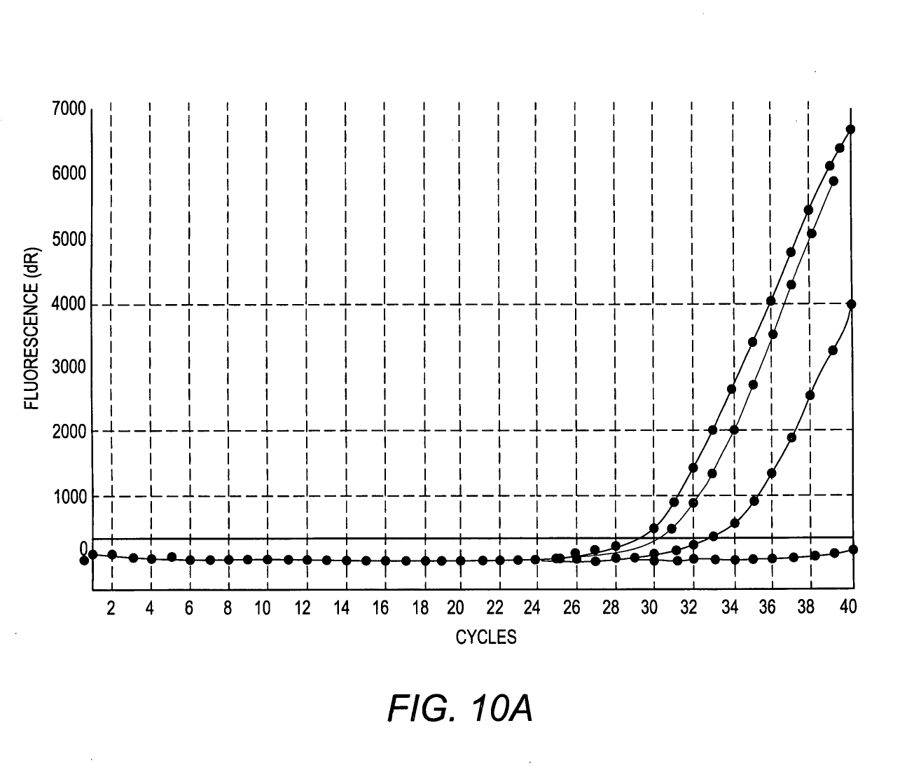

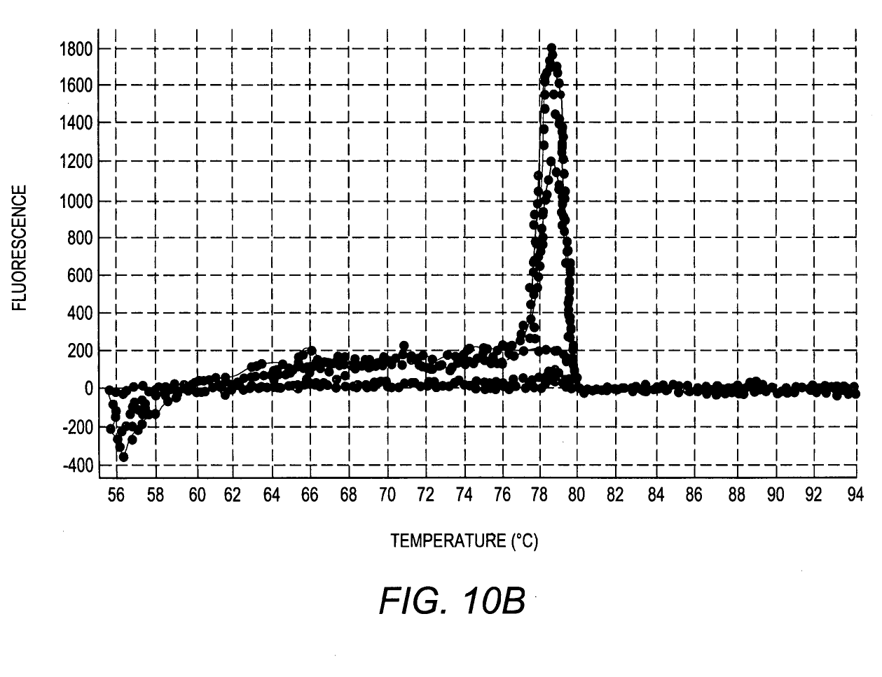

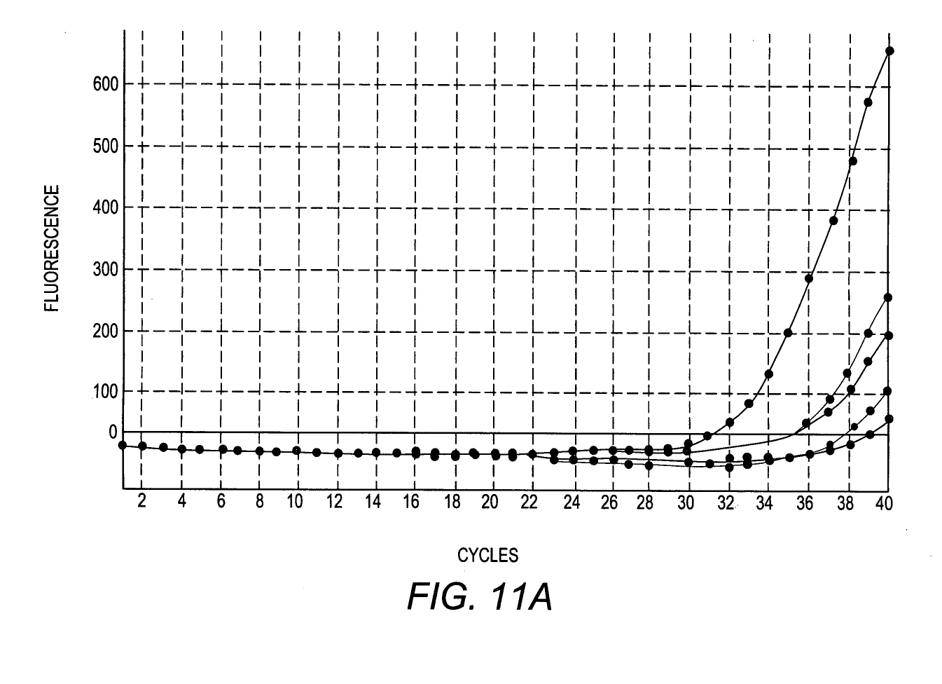

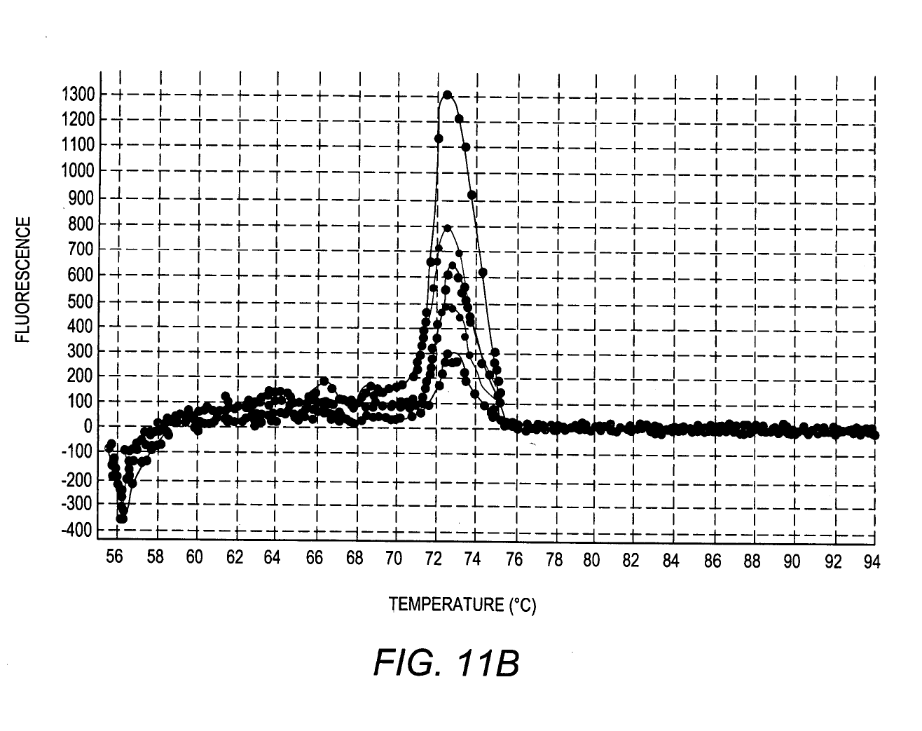

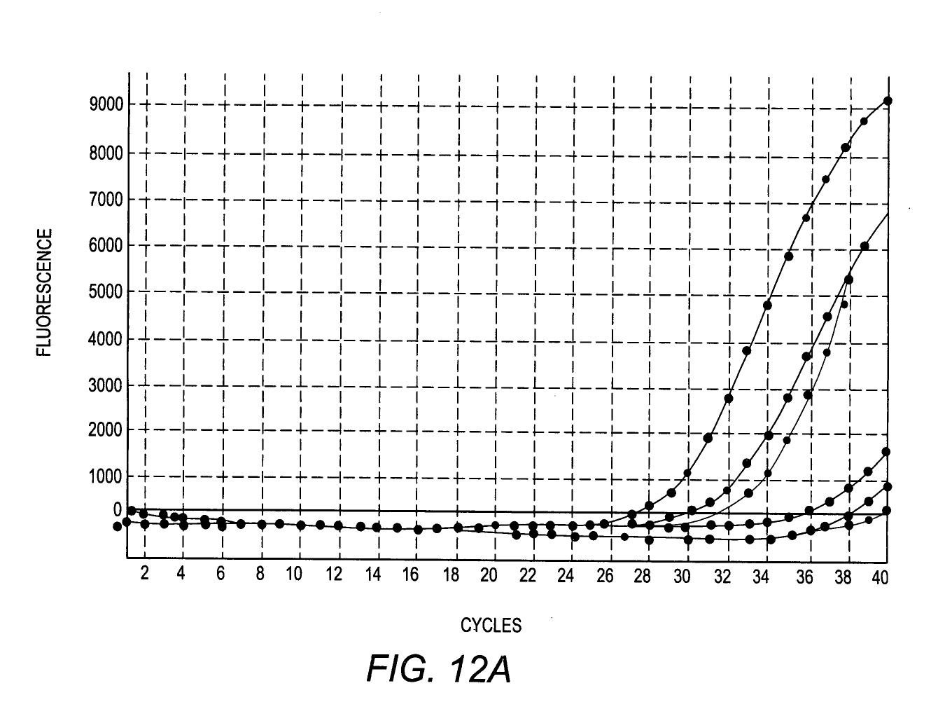

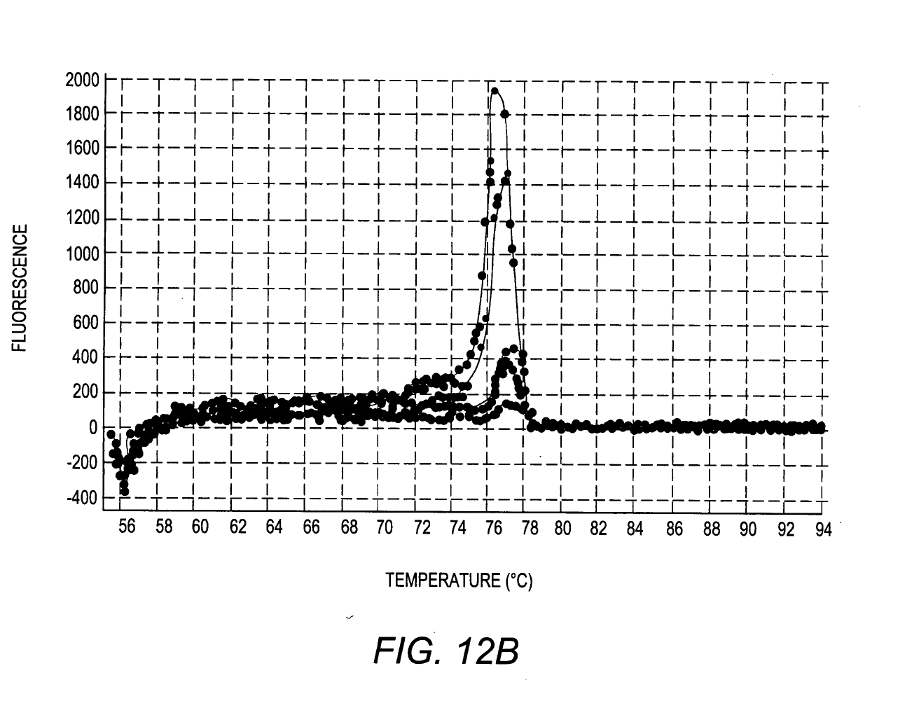

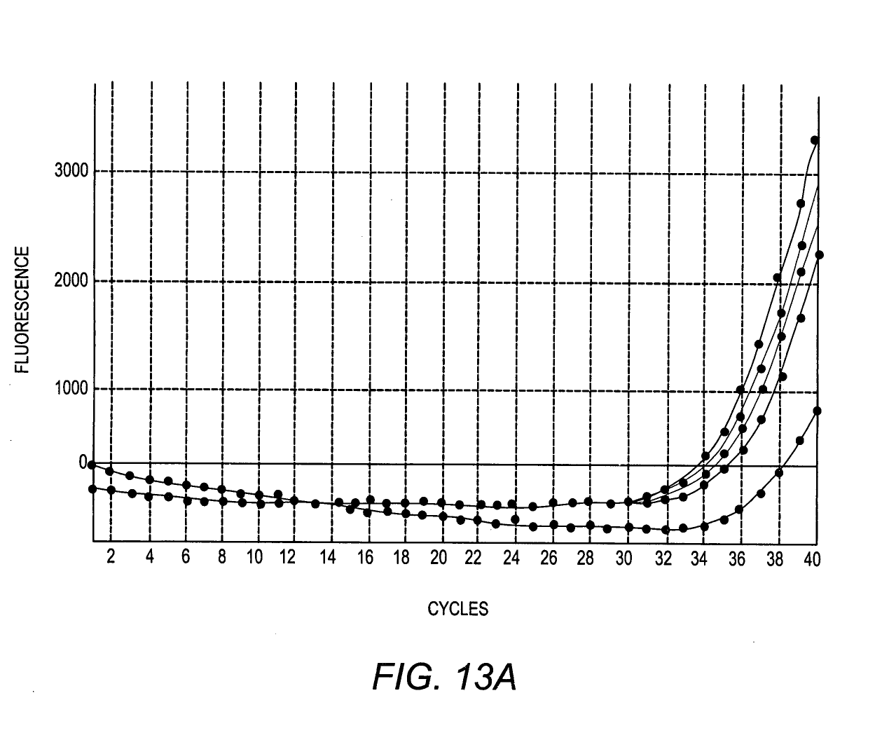

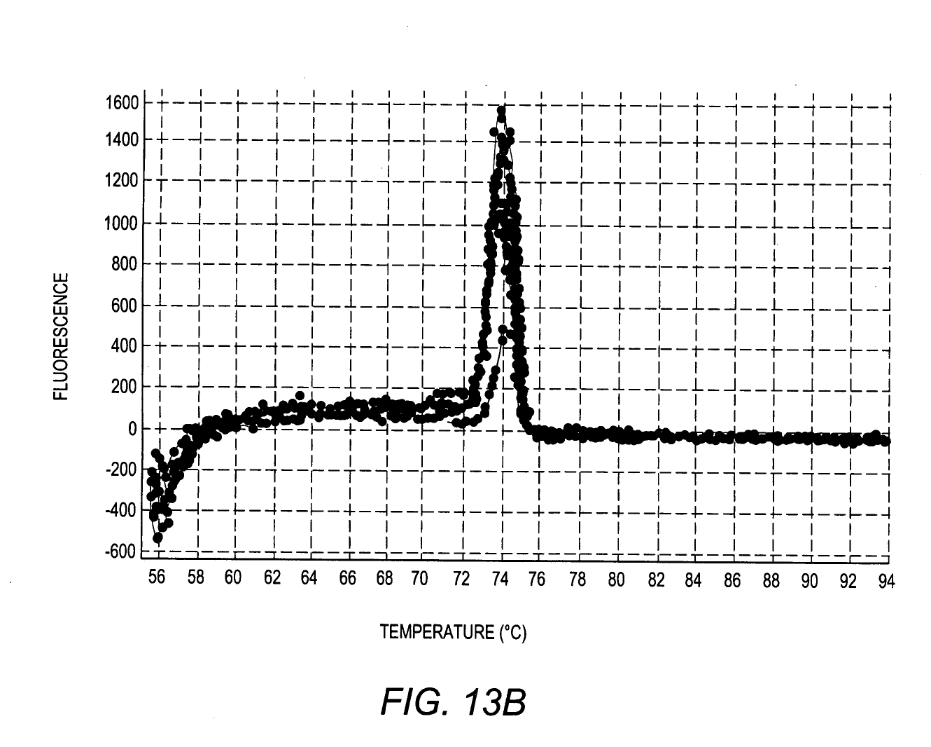

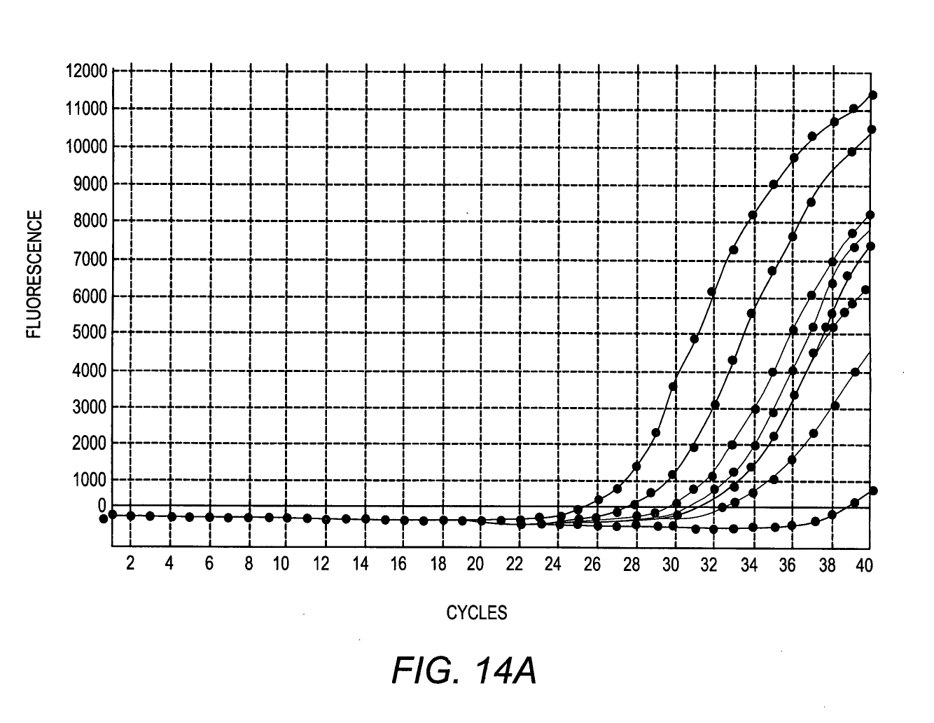

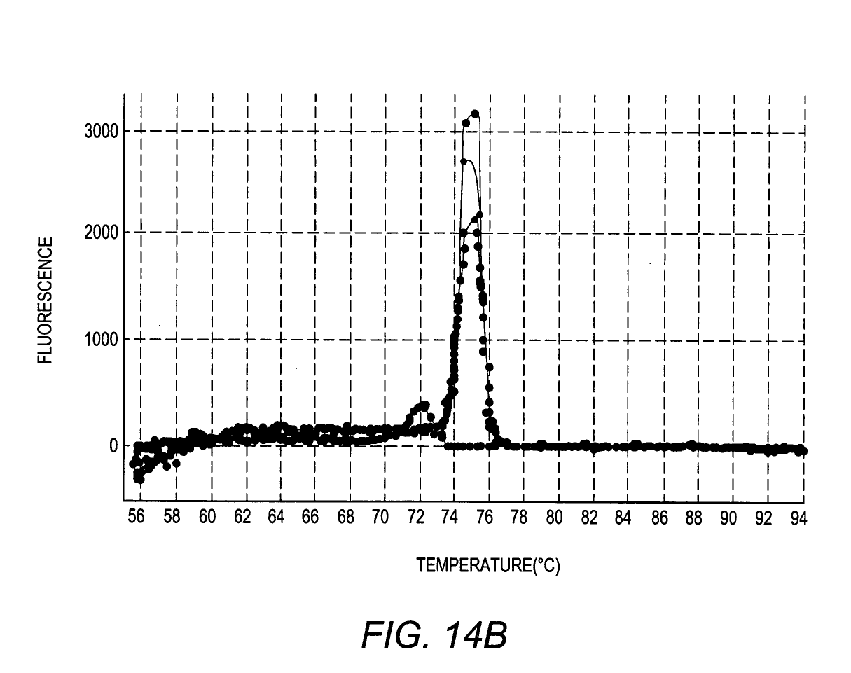

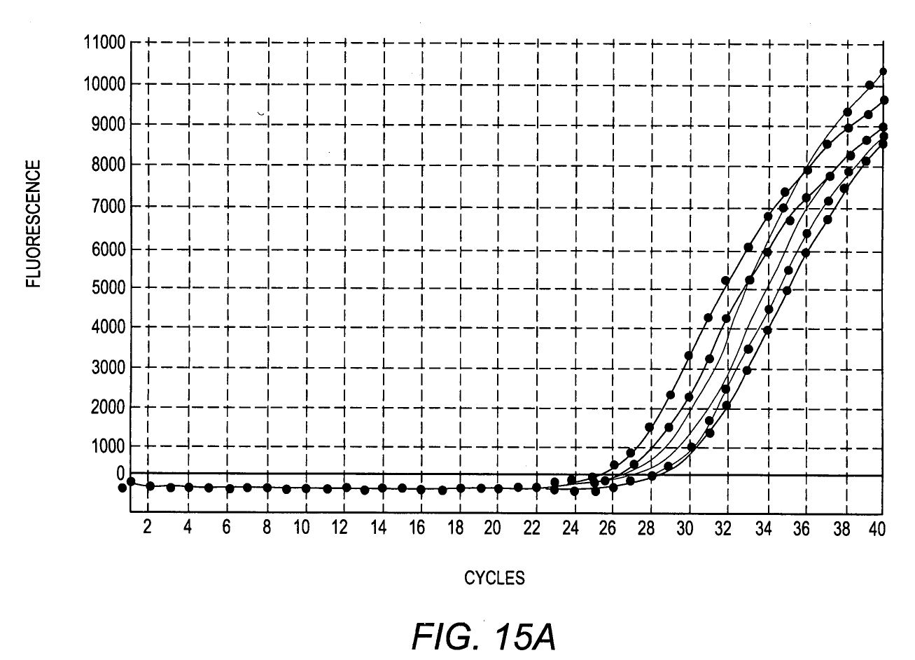

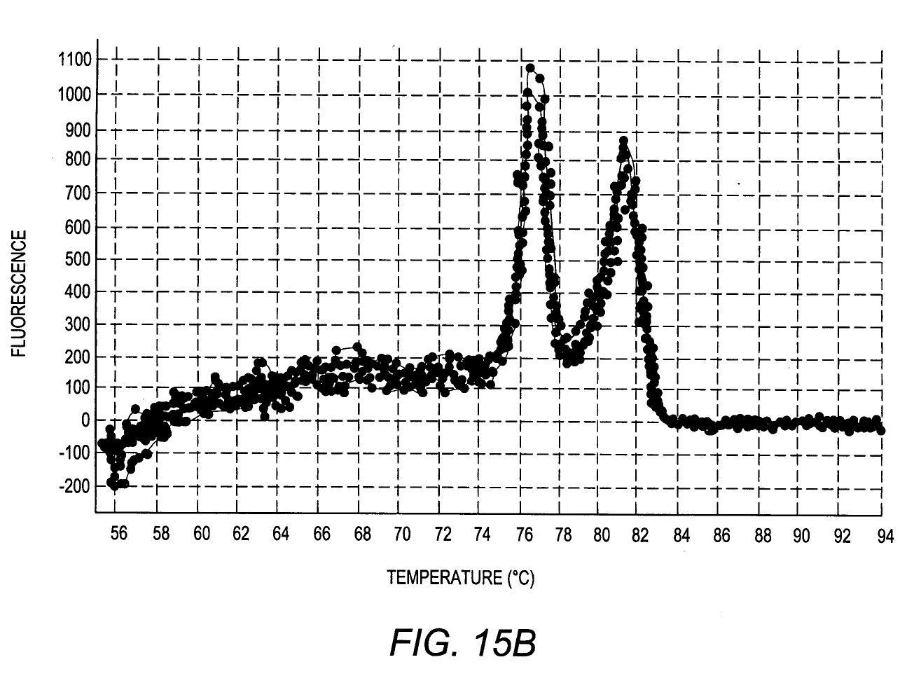

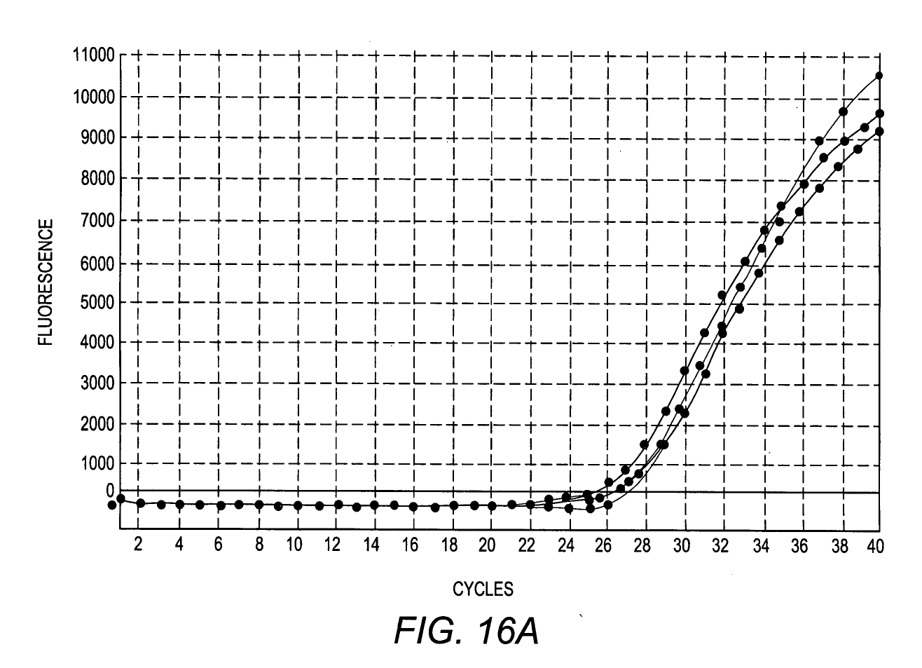

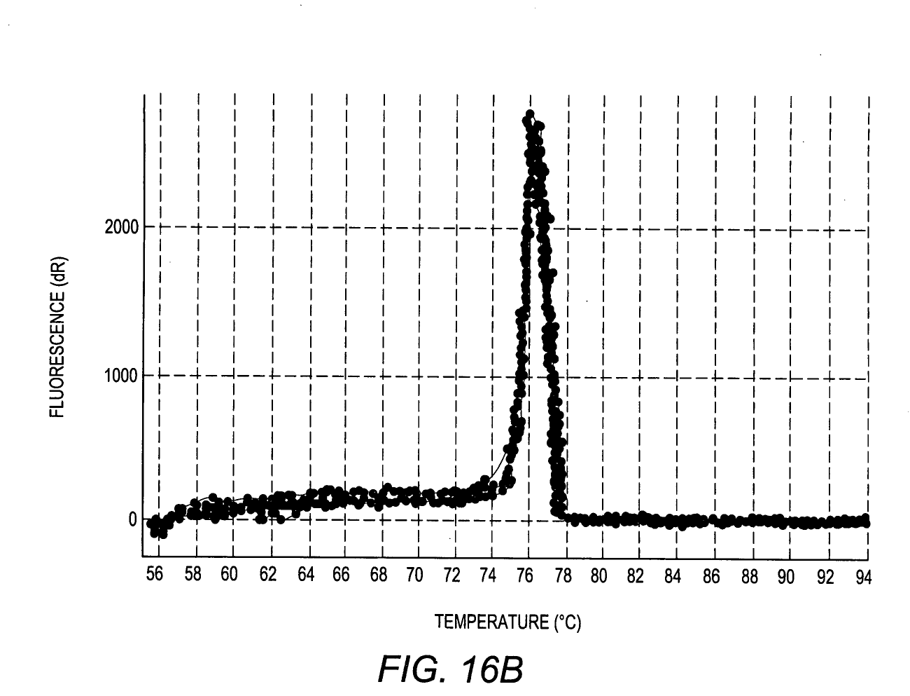

More specifically, FIG. 7A depicts the amplification profiles for the eight cell types using primer set #1 (SEQ ID NO:1 and SEQ ID NO:2); FIG. 8A depicts the amplification profiles for the eight cell types using primer set #2 (SEQ ID NO:3 and SEQ ID NO:4); FIG. 9A depicts the amplification profiles for the eight cell types using primer set #3 (SEQ ID NO:5 and SEQ ID NO:6); FIG. 10A depicts the amplification profiles for the eight cell types using primer set #4 (SEQ ID NO:7 and SEQ ID NO:8); FIG. 11A depicts the amplification profiles for the eight cell types using primer set #5 (SEQ ID NO:9 and SEQ ID NO:10); FIG. 12A depicts the amplification profiles for the eight cell types using primer set #6 (SEQ ID NO:11 and SEQ ID NO:12); FIG. 13A depicts the amplification profiles for the eight cell types using primer set #7 (SEQ ID NO:13 and SEQ ID NO:14); FIG. 14A depicts the amplification profiles for the eight cell types using primer set #8 (SEQ ID NO:15 and SEQ ID NO:16); FIG. 15A depicts the amplification profiles for the eight cell types using primer set #9 (SEQ ID NO:17 and SEQ ID NO:18); and FIG. 16A depicts the amplification profiles for the eight cell types using primer set #10 (SEQ ID NO:19 and SEQ ID NO:20). The corresponding dissociation curves are presented at FIGS. 7B, 8B, 9B, 10B, 11B, 12B, 13B, 14B, 15B, and 16B, respectively.

The results presented in FIGS. 7A through 16B show that all ten primer sets quantitatively amplify unique genomic sequences in human genomic DNA from at least one cell type. Indeed, primer sets 2 and 10 quantitatively amplify unique genomic sequences in human genomic DNA from multiple cell types. That is, primer sets 2 and 10 provide very repeatable Ct values among the eight cell types tested. This indicates that a single standard curve for some primer sets can be used across cell types, while other primer sets might require generation of a standard curve for one or more different cell types.

Accordingly, the invention provides a robust system of primers, compositions, methods, and kits for amplification and quantitation of genomic nucleic acids, based on amplification of unique sequence(s) in the genome of interest. The invention thus provides a powerful, accurate, and robust system for normalizing the amount of starting materials (e.g., cell lysates) for amplification reactions, which can provide highly accurate quantitation of the levels of target sequences of interest (e.g., an mRNA of interest) in those starting materials.

### Example 4

### Comparison of Amplification Effectiveness of gDNA With a Primer Set Using Two Different One-Step Lysis and Amplification Buffers

To determine the effectiveness of the primers of the invention in conjunction with “one-step” lysis and amplification buffers, primer set #10 (SEQ ID NO:19 and SEQ ID NO:20) was used to amplify gDNA from HeLa cells using two different buffers. One buffer was the commercially-available Cells to Signal II™ (Ambion; www.ambion.com) and a buffer according to the invention described in U.S. patent application Ser. No. 11/152,773, which is hereby incorporated herein by reference. The particular buffer according to the invention disclosed in that patent application that was used for the present experiments comprised 5 mM TCEP (Tris(2-carboxyethyl)phosphine) and 1% Triton X-100, pH 2.5.

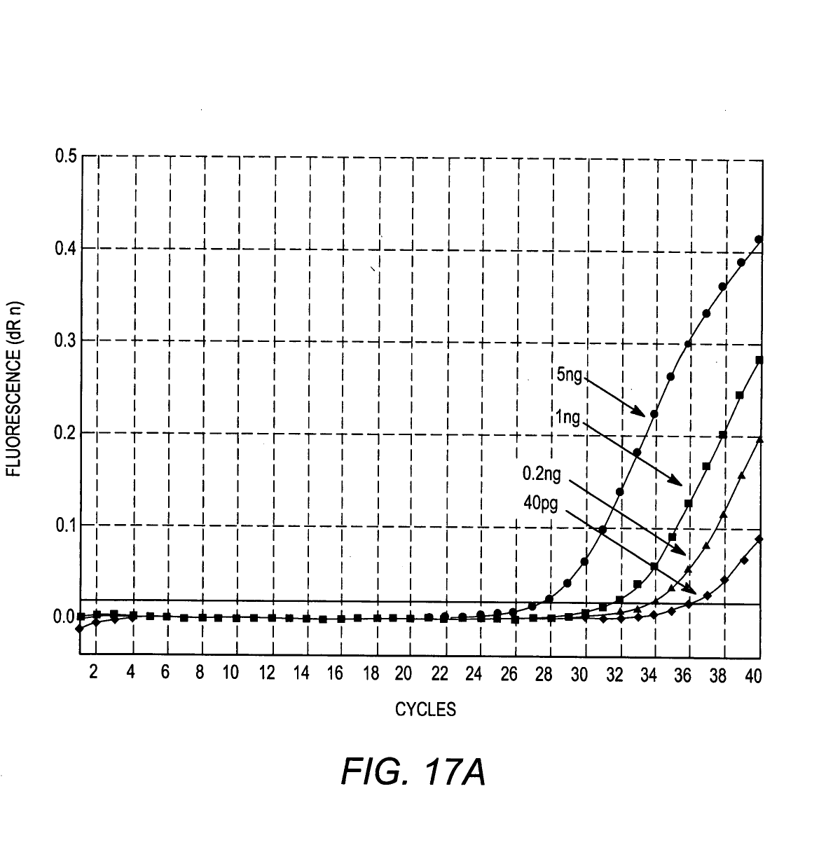

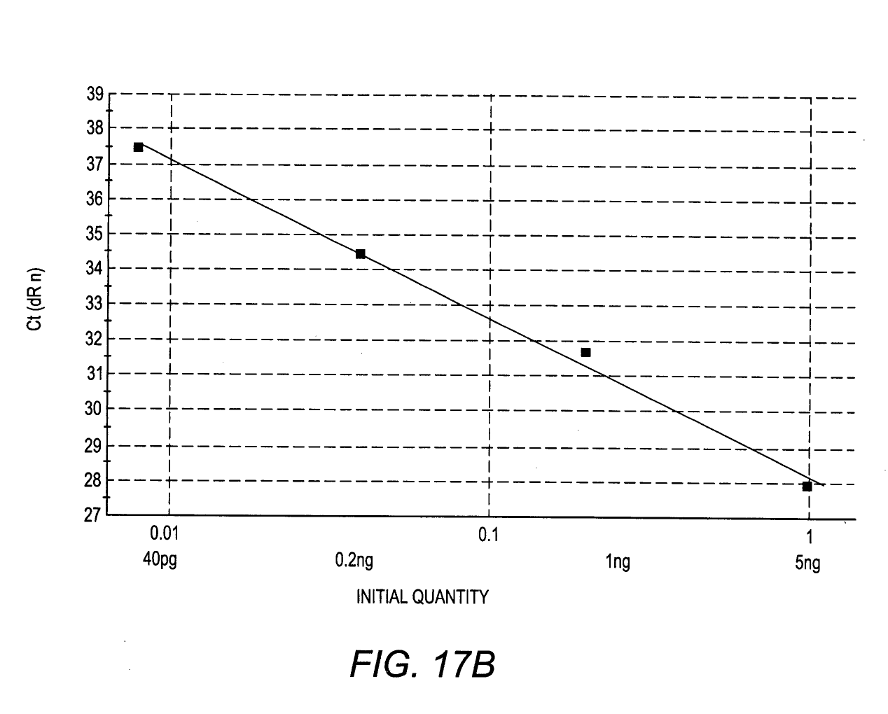

Initially, a standard curve of HeLa gDNA was generated from amplification plots from 40 pg, 200 pg, 1 ng, and 5 ng of DNA. Amplification was performed under the conditions described above in Example 1. Amplification plots of the reactions are depicted in FIG. 17A. The standard curve from the amplification plots is depicted in FIG. 17B. As can be seen from these Figures, amplification of the gDNA was robust along the concentrations used, resulting in a standard curve with a straight line.

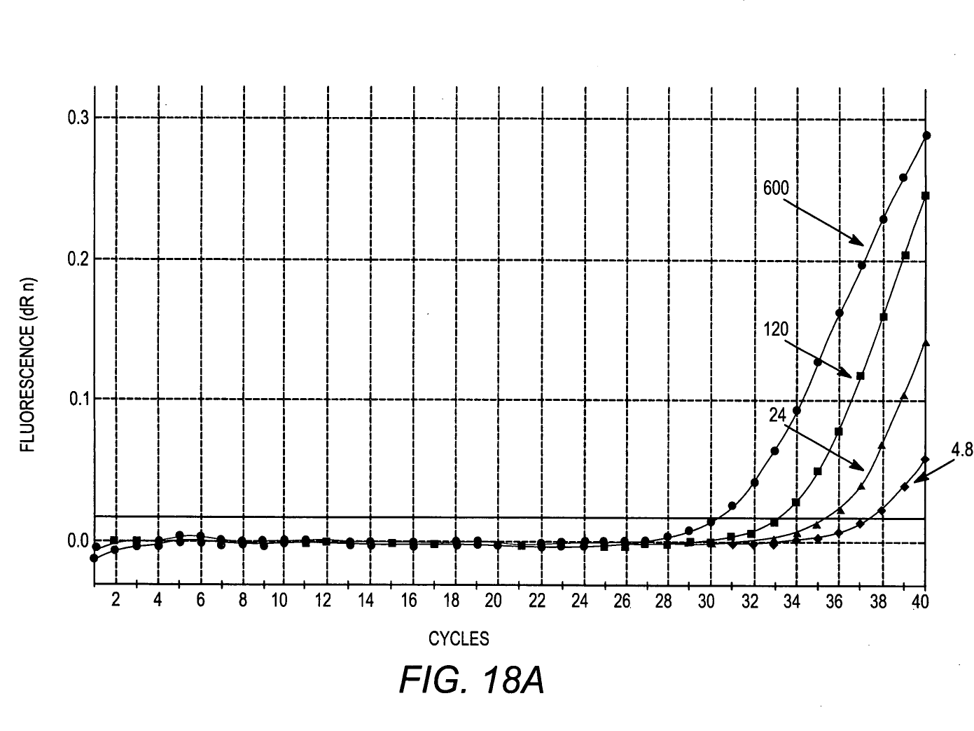

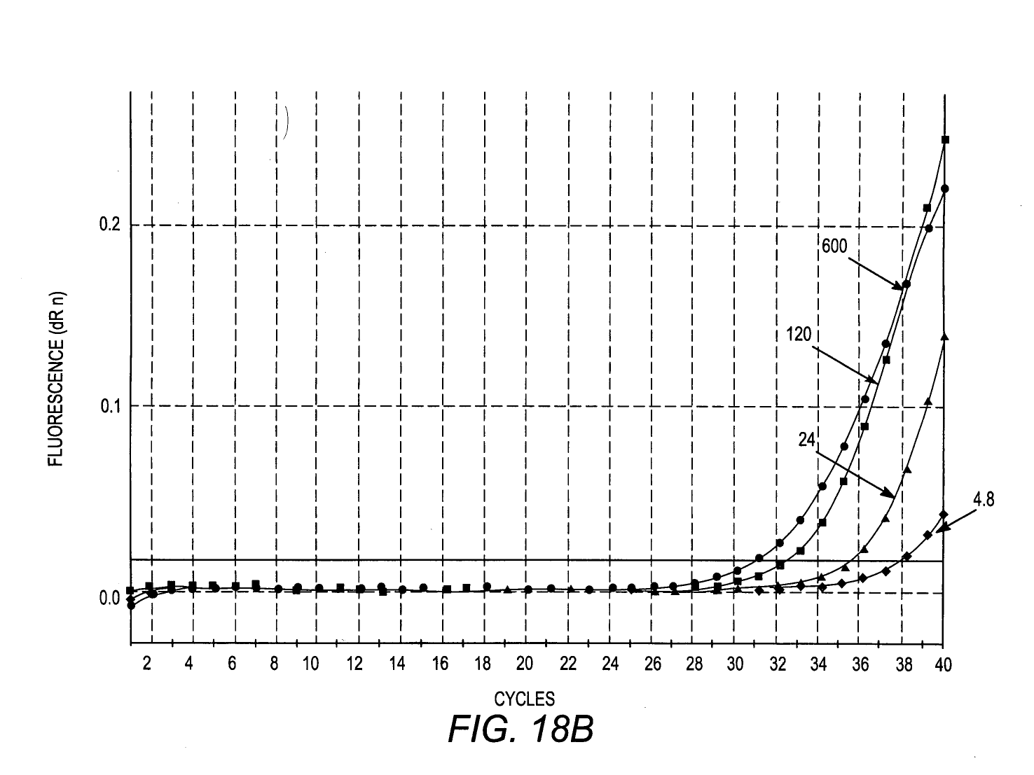

Additional amplification reactions were performed to determine the effectiveness of the primer set when used in conjunction with the two “one-step” buffers and cell lysates representing different numbers of cells. The amplification reactions comprised either 3 ul of cell lysate in a one-step buffer or 5 ul of cell lysate in a one-step buffer. The number of cells represented by the cell lysate samples ranged from 4.8 cells to 1,000 cells. The results of the amplification reactions are depicted in FIGS. 18A through 21.

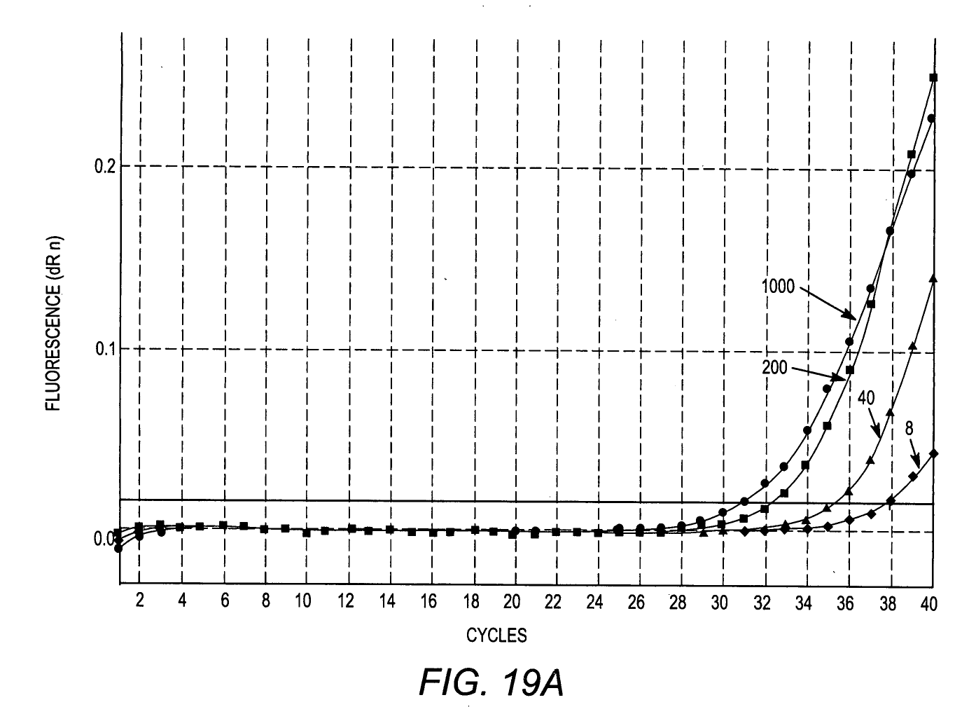

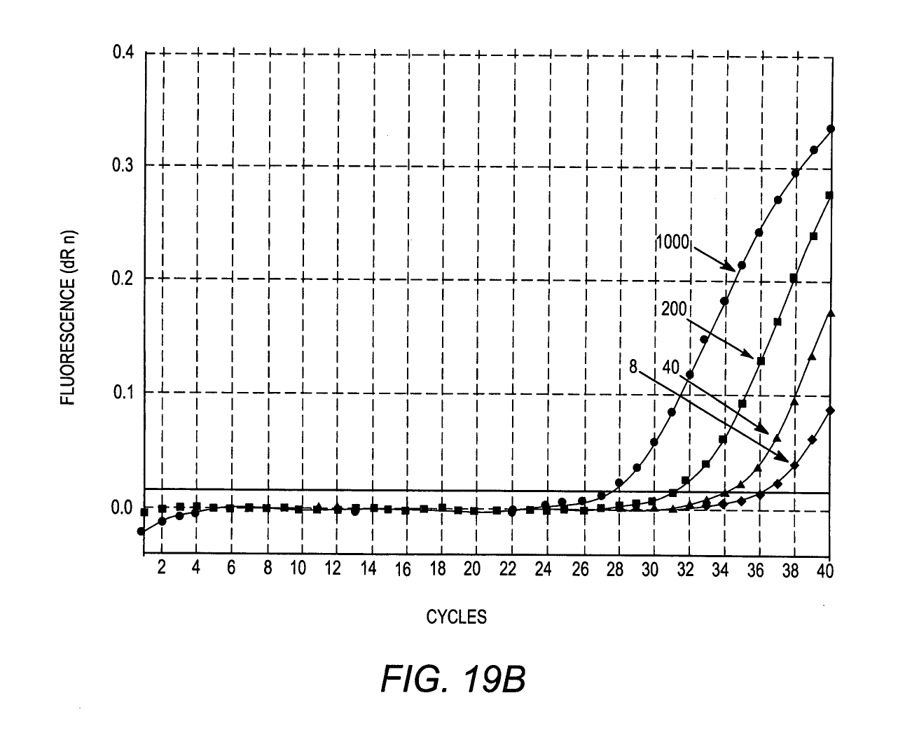

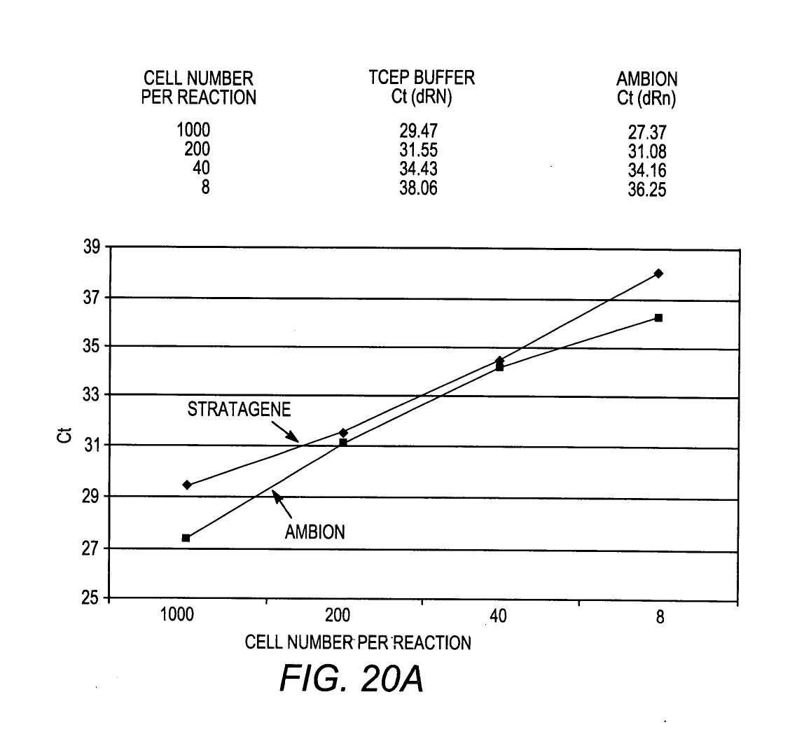

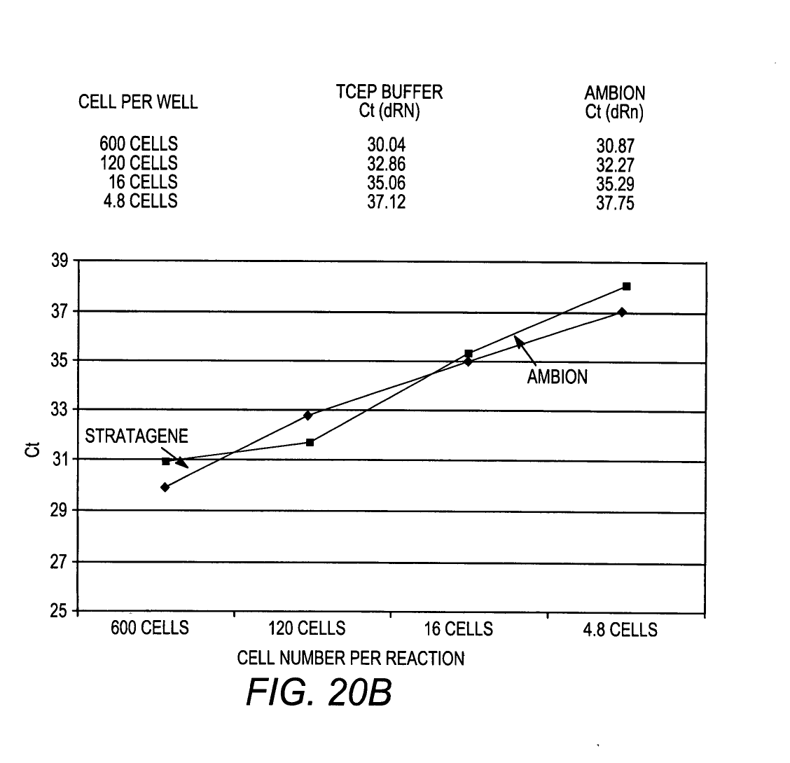

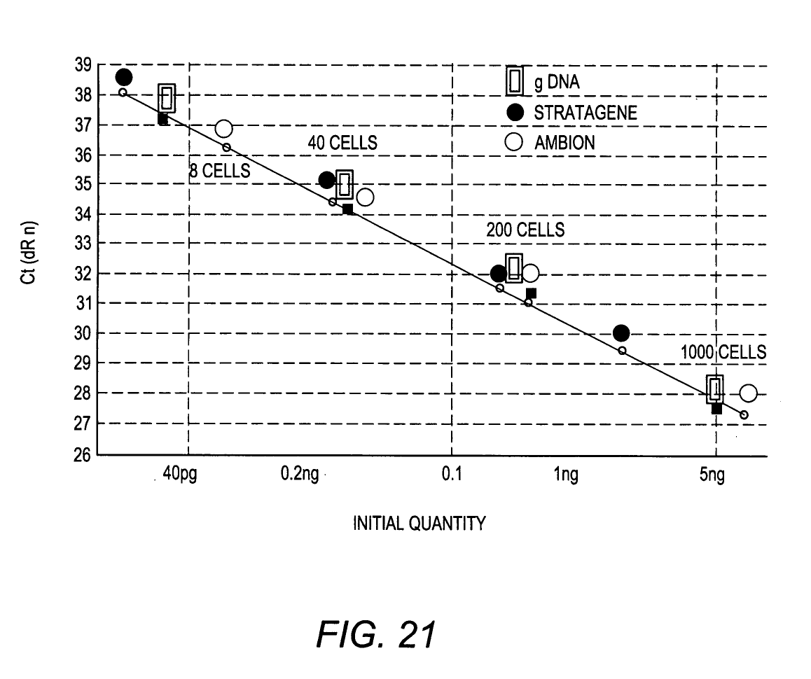

More specifically, FIG. 18A depicts the amplification plots of cell lysates created in a buffer comprising 5 mM TCEP, 1% Triton X-100, pH 2.5, where the plots represent amplification of cell lysates from 4.8 cells, 24 cells, 120 cells, and 600 cells. FIG. 18B depicts the amplification plots of the same amounts of cell lysates (and thus cells) as in FIG. 18A, but performed using the Ambion Cells To Signal II™ buffer. FIG. 19A depicts the amplification plots of cell lysates created in a buffer comprising 5 mM TCEP, 1% Triton X-100, pH 2.5, where the plots represent amplification of cell lysates from 8 cells, 40 cells, 200 cells, and 1,000 cells. FIG. 19B depicts the amplification plots of the same amounts of cell lysates (and thus cells) as in FIG. 19A, but performed using the Ambion Cells To Signal II™ buffer. FIG. 20A depicts standard curves created from the data presented in FIGS. 18A and 18B. FIG. 20B depicts standard curves created from the data presented in FIGS. 19A and 19B. FIG. 21 presents a summation of the data presented in FIGS. 18A through 20B.

The results presented in these Figures show that amplification of gDNA in these two buffers can be accomplished using a primer set of the present invention. It also indicates that amplification profiles vary linearly with amount of genomic nucleic acid supplied, either as purified gDNA or as cell lysate resulting from lysis of cells using the two “one-step” buffers. In addition, the data shows that the amount of cells from which a cell lysate is obtained can be determined using the primers, compositions, methods, and kits of the present invention. Accordingly, cell samples can be normalized for amounts of starting materials, and valid, accurate conclusions regarding the absolute or relative amounts of various nucleic acid targets (e.g., expression products) may be drawn.

### Example 5

### Comparison of QRT-PCR and QPCR Amplification With Primers of the Invention and a TaqMan® Primer/Probe Set

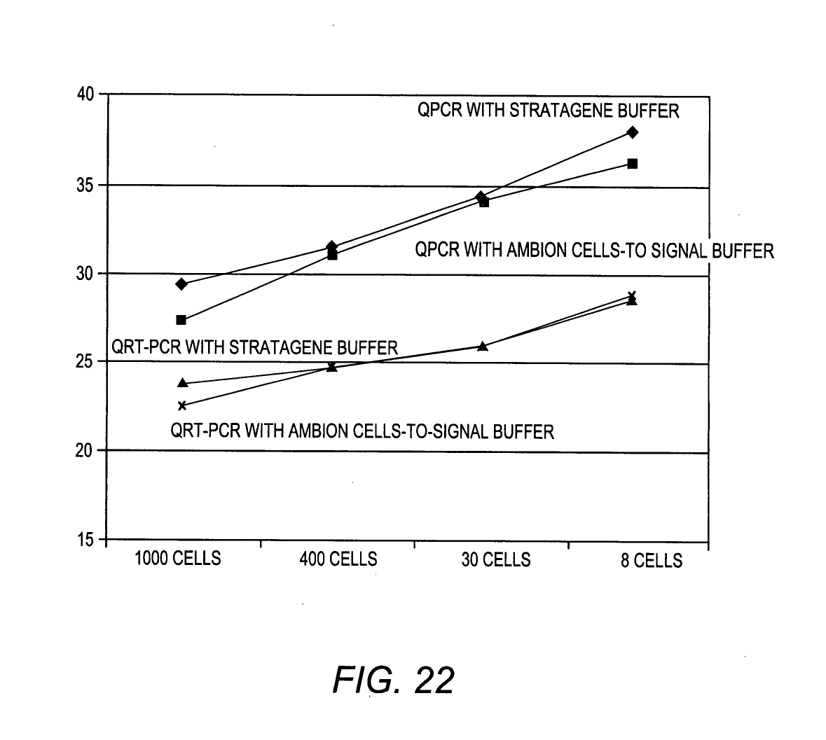

To further show the applicability of primer sets of the invention, primer set 10 (SEQ ID NO:19 and SEQ ID NO:20) were used to track gDNA concentration in conjunction with TaqMan® amplification of a target sequence. More specifically, varying amounts of HeLa cells (1000 cells, 400 cells, 30 cells, 8 cells) were lysed with 5 ul of either a buffer comprising 5 mM TCEP, 1% Triton X-100, pH 2.5 (“Stratagene Buffer”) or the buffer supplied in the Ambion “Cells-to-Signal” kit. Primer set 10 was used to evaluate gDNA concentration in each sample using the SYBR® Green detection system. Detection of target sequences on mRNA in the same samples was performed using the GAP TaqMan® primer/probe set of Ambion in a 1-step QRT-PCR protocol. The results of amplification reactions are shown in FIG. 22, which plots Ct values for the various amplification curves versus numbers of cells in the original sample.

As can be seen from FIG. 22, the primer set of the present invention can be used to quantitatively detect unique gDNA sequences in a complex mixture, such as cell lysates. Furthermore, the primer set is functional in two distinct lysis buffers. The primers quantitatively detect gDNA present in the cell lysates, and provide a convenient, internal, benchmark against which samples can be compared to normalize the amount of nucleic acid, and thus the amount of original starting material (e.g., the number of cells) analyzed between samples.

### Example 6

### QPCR Amplification With HeLa Cells Lysate

HeLa cells at a concentration of 10,000 cells/ul were lysed in a buffer comprising 5 mM TCEP and 1% Triton X-100, pH 2.5. After cells were lysed in the buffer, two-fold serial dilutions were made in TE buffer (pH 7.0) and 1 ul of each dilution was used in QPCR or QRT-PCR with 25-ul reaction volume.

QPCR was carried out using Brilliant® SYBR® Green QPCR Master Mix (Stratagene Cat. No. 600548) and the DNA-specific primer set 10 (SEQ ID NO:19 and SEQ ID NO:20) on the Mx3000P Real-Time PCR System (Stratagene). PCR amplifications were performed as follows: 1 cycle of 10 minutes at 95° C.; 40 cycles comprising 15 seconds at 95° C., and 1 minute at 60° C.

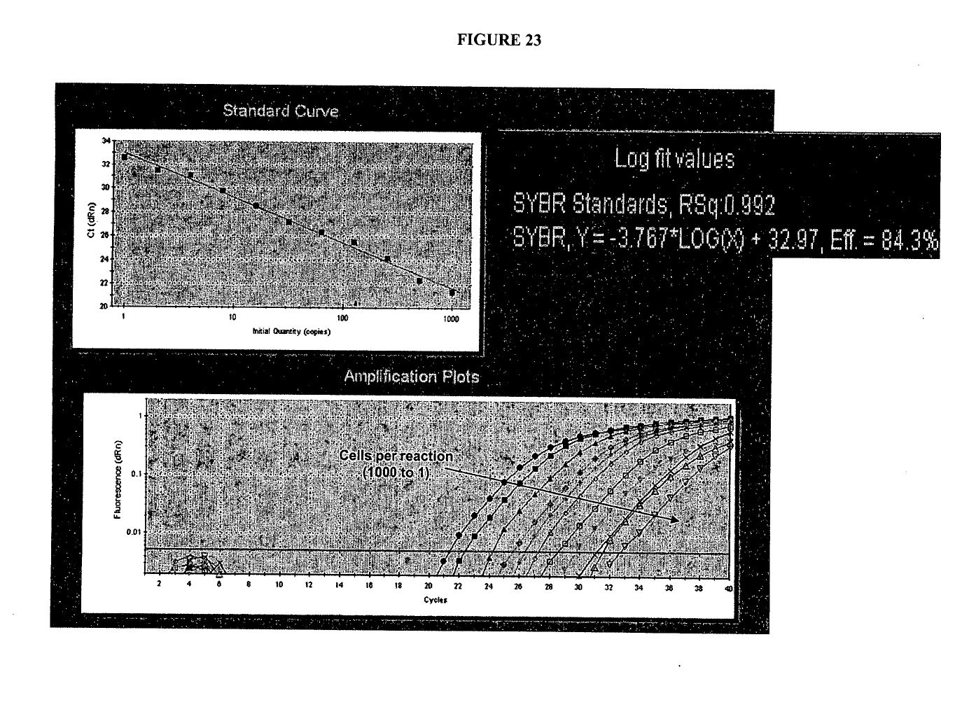

The standard curve for amplification of HeLa gDNA with primer set 10 (SEQ ID NO:19 and SEQ ID NO:20), which plots Ct vs. initial quantity (relative numbers) of HeLa gDNA in the reaction, is presented in FIG. 23 (top). The standard curve presented in FIG. 23 shows that the amplification reactions, as evaluated by Ct values, are linear across at least 3 orders of magnitude, from cells numbers of 1 or less up to 1000 or more. The amplification plots are shown in FIG. 23 (bottom). The standard curve in FIG. 23 demonstrates a linear amplification with about 84% efficiency.

### Example 7

### QRT-PCR Amplification With HeLa Cells Lysate

HeLa cells at a concentration of 10,000 cells/ul were lysed in the buffer described in Example 6. QRT-PCR was performed using Brilliant® QRT-PCR Master Mix and RNA-specific B2M TaqMan® primers and probes (Assay on Demand, ABI) using a one-step QRT-PCR protocol on the Mx3000P Real-Time PCR System (Stratagene). PCR amplifications were performed as follows: 1 cycle of 30 minutes at 50° C.; 1 cycle of 10 minutes as 95° C.; 40 cycles comprising 15 seconds at 95° C., and 1 minute at 60° C.

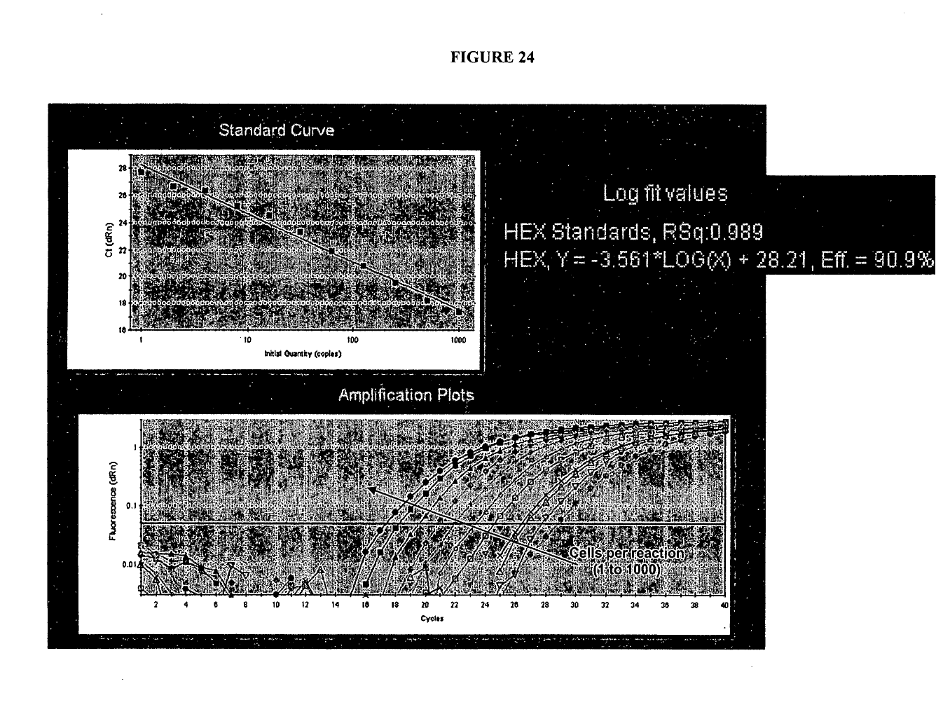

The standard curve for amplification for B2M mRNA with RNA-specific B2M TaqMan® primers and probes is presented in FIG. 24 (top). This standard curve shows that the amplification reactions, as evaluated by Ct values, are linear across at least 3 orders of magnitude, from cells numbers of 1 or less up to 1000 or more. The amplification plots are shown in FIG. 24 (bottom). The standard curve in FIG. 24 demonstrates a linear amplification with about 91% efficiency.

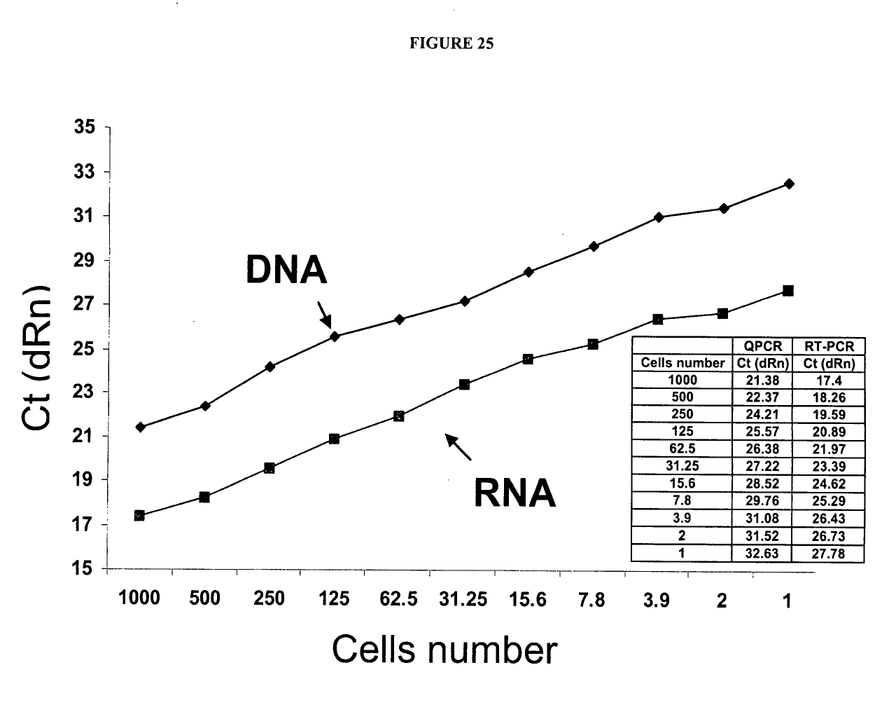

FIG. 25 presents a summary of the data in Examples 6 and 7, comparing the amplification reactions after two-fold serial dilutions of HeLa cell lysate. The amplification reactions are evaluated by Ct values, between DNA-specific primer set 10 (SEQ ID NO:19 and SEQ ID NO:20) in QPCR (upper curve) and the RNA-specific B2M TaqMan® primers and probes in one-step QRT-PCR (lower curve). The Ct data is also presented in tabular format in the inset of FIG. 25. The data presented in FIG. 25 indicates that there is a very good correlation between DNA and RNA amounts dependent on the number of cells per reaction.

### Example 8

### QPCR Amplification With Human Liver Lysate

Human liver tissue was homogenized in the buffer described in earlier Examples. After cells were homogenized in the buffer, ten-fold serial dilutions were made in the buffer and 1 ul of each dilution was used in QPCR or QRT-PCR with 25-ul reaction volume.

QPCR was carried out using Brilliant® SYBR® Green QPCR Master Mix (Stratagene Cat. No. 600548) and the DNA-specific primer set 10 (SEQ ID NO:19 and SEQ ID NO:20) on the Mx3000P Real-Time PCR System (Stratagene). PCR amplifications were performed as follows: 1 cycle of 10 minutes at 95° C.; 40 cycles comprising 15 seconds at 95° C., and 1 minute at 60° C.

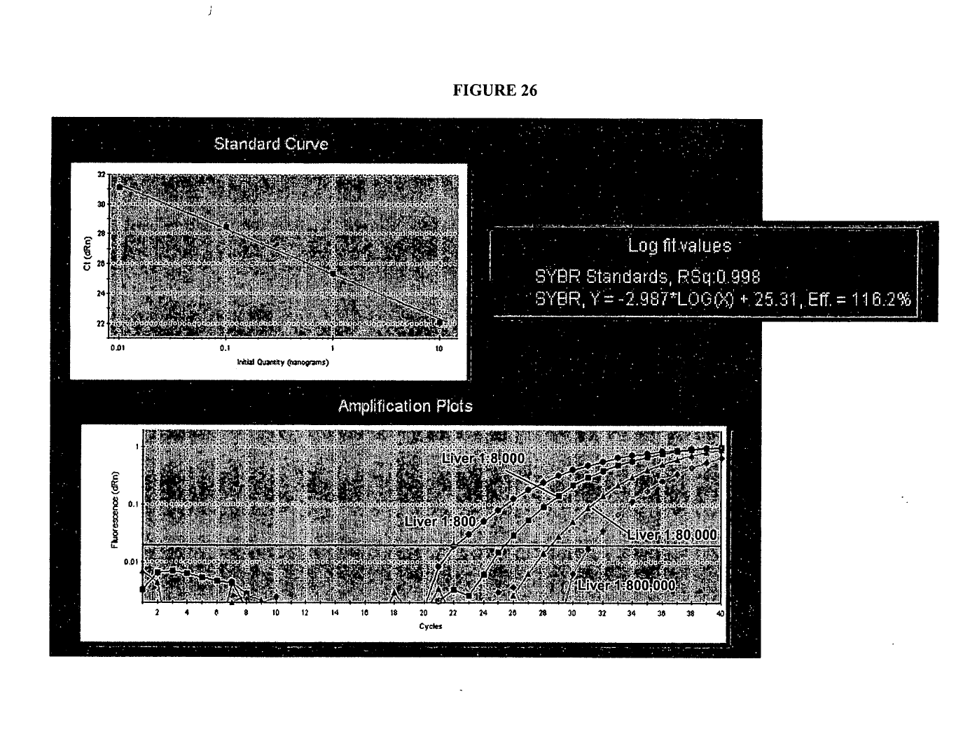

The standard curve for amplification of human liver tissue lysate with primer set 10 (SEQ ID NO:19 and SEQ ID NO:20), which plots Ct vs. initial quantity (relative numbers) of liver lysate in the reaction, is presented in FIG. 26 (top). The standard curve presented in FIG. 26 shows that the amplification reactions, as evaluated by Ct values, are linear across at least 3 orders of magnitude. The amplification plots are shown in FIG. 26 (bottom). The standard curve in FIG. 26 demonstrates a linear amplification with about 116% efficiency.

### Example 9

### QRT-PCR Amplification With Human Liver Lysate

Human liver tissue was homogenized in the buffer as described above. QRT-PCR was performed using Brilliant® QRT-PCR Master Mix and RNA-specific B2M TaqMan® primers and probes (Assay on Demand, ABI) using a one-step QRT-PCR protocol on the Mx3000P Real-Time PCR System (Stratagene). PCR amplifications were performed as follows: 1 cycle of 30 minutes at 50° C.; 1 cycle of 10 minutes as 95° C.; 40 cycles comprising 15 seconds at 95° C., and 1 minute at 60° C.

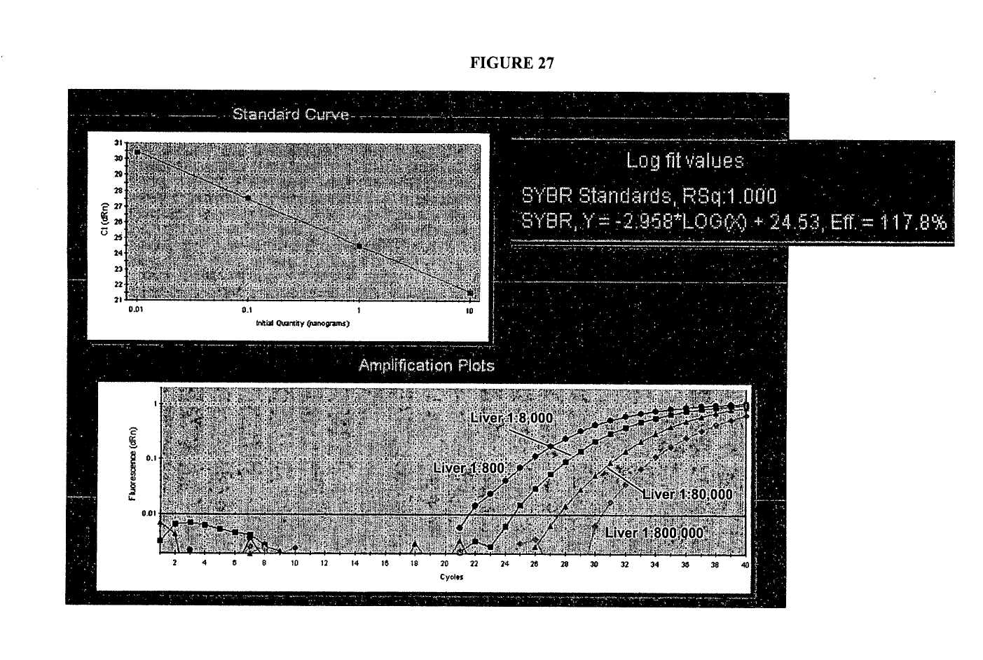

The standard curve for amplification of human liver tissue lysate with RNA-specific B2M TaqMan® primers and probes is presented in FIG. 27 (top). This standard curve shows that the amplification reactions, as evaluated by Ct values, are linear across at least 3 orders of magnitude, from initial quantities of 0.01 ng or less up to 10 ng or more. The amplification plots are shown in FIG. 27 (bottom). The standard curve in FIG. 27 demonstrates a linear amplification with about 118% efficiency.

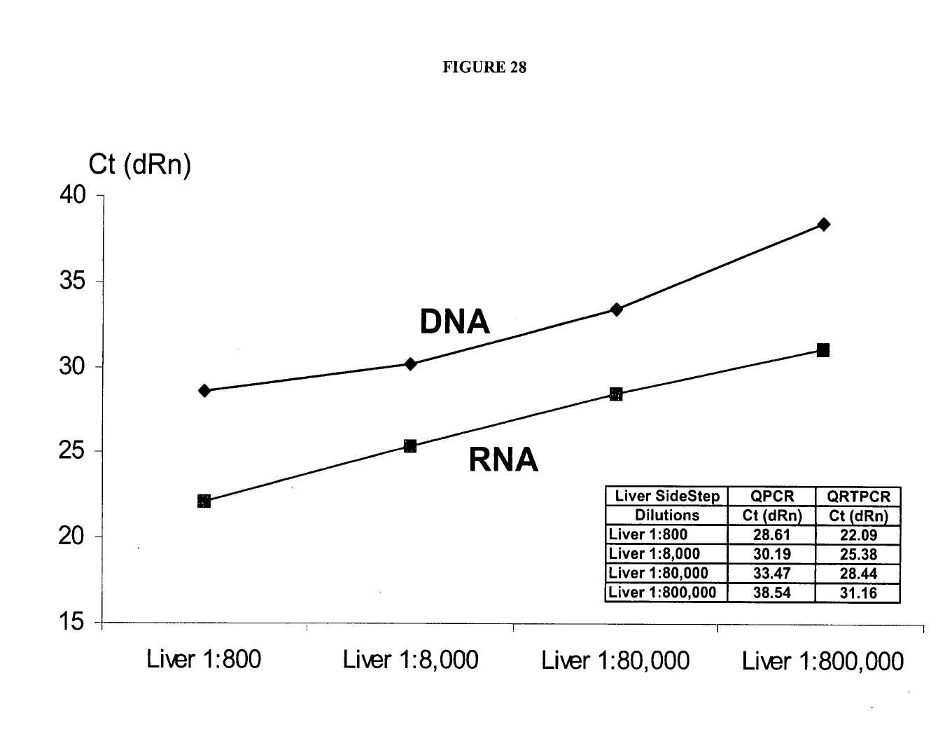

FIG. 28 presents a summary of the data in Examples 8 and 9, comparing the amplification reactions after ten-fold serial dilutions of human liver tissue lysate. The amplification reactions are evaluated by Ct values, between DNA-specific primer set 10 (SEQ ID NO:19 and SEQ ID NO:20) in QPCR (upper curve) and the RNA-specific B2M TaqMan® primers and probes in one-step QRT-PCR (lower curve). The Ct data is also presented in tabular format in the inset of FIG. 28. The data presented in FIG. 28 indicates that there is a good correlation between DNA and RNA amounts.

### Example 10

### QPCR and QRT-PCR Amplification With HeLa Cells Lysate

HeLa cells at different concentrations were lysed in the buffer from above. The concentrations were about 100, 20, 4, or 0.8 cells/ul.

QPCR was carried out using Brilliant® SYBR® Green QPCR Master Mix (Stratagene Cat. No. 600548) and the DNA-specific primer set 10 (SEQ ID NO:19 and SEQ ID NO:20) on the Mx3000P Real-Time PCR System (Stratagene). QPCR amplifications were performed as follows: 1 cycle of 10 minutes at 95° C.; 40 cycles comprising 15 seconds at 95° C., and 1 minute at 60° C. QRT-PCR was performed using Brilliant® QRT-PCR Master Mix and TaqMan® primers and probes (BAX, USP7, and B2M; Assay on Demand, ABI) using a one-step QRT-PCR protocol on the Mx3000P Real-Time PCR System (Stratagene). QRT-PCR amplifications were performed as follows: 1 cycle of 30 minutes at 50° C.; 1 cycle of 10 minutes as 95° C.; 40 cycles comprising 15 seconds at 95° C., and 1 minute at 55° C.

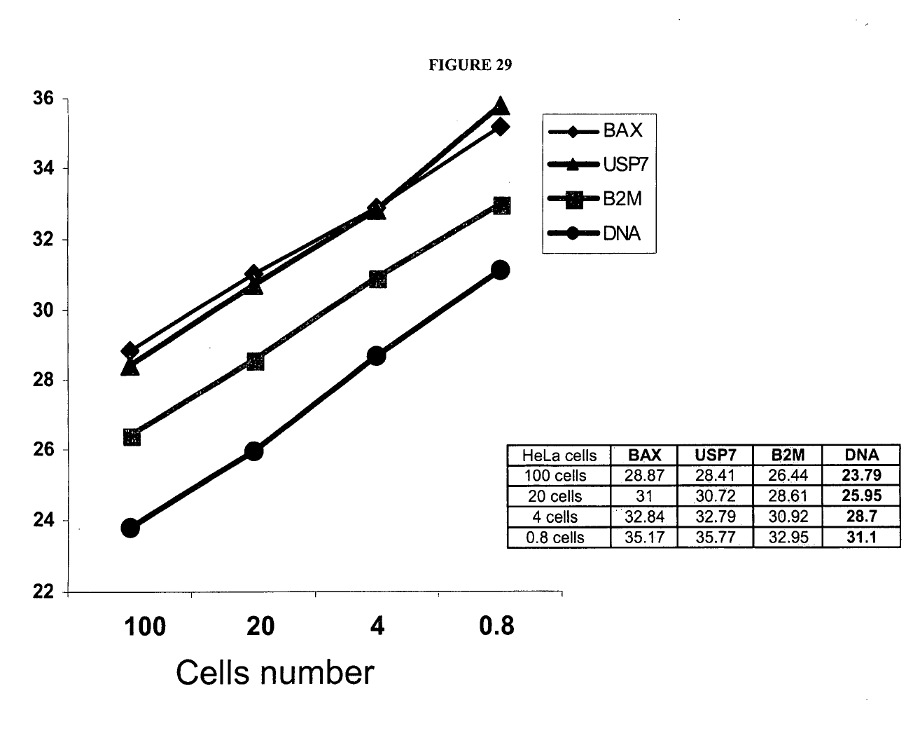

FIG. 29 compares the amplification reactions at the four different cell concentrations employed in this example. The amplification reactions are evaluated by Ct values as a function of cell number (cells per ul), comparing DNA-specific primer set 10 (SEQ ID NO:19 and SEQ ID NO:20) in QPCR to BAX, USP7, and B2M RNA-specific TaqMan® primers and probes in one-step QRT-PCR. FIG. 29 demonstrates a very good correlation between DNA and RNA amounts dependent on the number of cells per reaction.

### Example 11

### Comparative Quantification of BAX Gene Expression in HeLa Cells Using B2M or DNA as the Normalizer

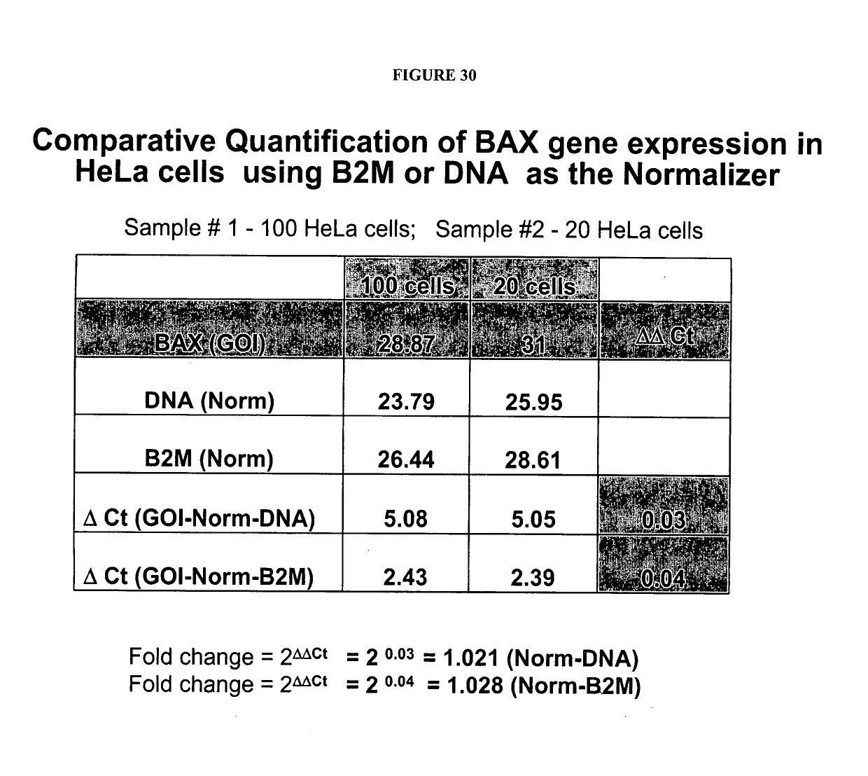

QPCR and QRT-PCR were carried out as described in Example 10, for sample 1 comprising 100 HeLa cells and sample 2 comprising 20 HeLa cells. Gene expression of BAX was compared in samples 1 and 2. The amplification reactions are evaluated by Ct values. For BAX amplification, the Ct values were about 28.9 for 100 cells and about 31.0 for 20 cells (as depicted in FIG. 30). The normalizer comprised either DNA-specific primer set 10 (SEQ ID NO:19 and SEQ ID NO:20) in QPCR or RNA-specific B2M TaqMan® primers and probes in one-step QRT-PCR. The Ct values for the two normalizers at either cell concentration are shown in FIG. 30. ΔΔCt was calculated using either DNA or B2M as the normalizer and demonstrated similar results (ΔΔCt=0.03 or 0.04, respectively).

The data in this example demonstrates that single-copy gDNA can be successfully used as the normalizer in comparative quantification analysis of gene expression.

It will be apparent to those skilled in the art that various modifications and variations can be made in the practice of the present invention without departing from the scope or spirit of the invention. Other embodiments of the invention will be apparent to those skilled in the art from consideration of the specification and practice of the invention. It is intended that the specific disclosure of the specification be considered as exemplary only, with a true scope and spirit of the invention being indicated by the following claims.

1. A composition comprising:
at least one cell lysate,
at least one primer,
at least one probe for detection of a unique genomic nucleic acid sequence, a target nucleic acid of interest, or both, and
one or more components for a PCR reaction.

2. The composition of claim 1, wherein the components for a PCR reaction comprise at least one thermostable polymerase.

3. The composition of claim 1, where the components for a PCR reaction comprise at least one reverse transcriptase.

4. A method for determining the amount of gDNA, the number of cells present, or the cell concentration, in a composition from which a test sample is derived, said method comprising:
providing the test sample comprising genomic nucleic acid,
amplifying one or more unique genomic sequences present in the genomic nucleic acid,
comparing an amplification profile of the amplified genomic nucleic acid to a standard curve of amplification profiles obtained from reactions performed on reference samples from known amount of gDNA or numbers of original cells of the type from which the genomic nucleic acid originates, and
determining the amount of gDNA or the number of cells or the cell concentration from which the test sample genomic nucleic acids originated.

5. The method of claim 4, wherein the method is performed on more than one sample.

6. The method of claim 5, further comprising normalizing the multiple samples based on the amount of gDNA or the number of cells or the cell concentration determined through the method.

7. The method of claim 5, further comprising amplifying one or more target sequences of interest, which is different from the unique genomic sequence(s).

8. The method of claim 5, which is a PCR method for determining the number or concentration of cells from one or more test samples, and wherein said method comprises normalizing the number or concentration obtained by PCR amplification, wherein normalizing uses at least one unique genomic sequence present in the genomic nucleic acid.

9. A kit for determining the amount of gDNA or the number of cells present, or the cell concentration, in a composition from which a test sample is derived, said kit comprising:
at least one cell lysate,
at least one primer,
at least one probe for detection of a unique genomic nucleic acid sequence, a target nucleic acid of interest, or both, and
one or more components for a PCR reaction.

10. The kit of claim 9, wherein at least one primer is an oligonucleotide primer for amplification of a target sequence of interest, which is a sequence other than a unique genomic sequence.

11. The kit of claim 9, further comprising some or all of the components necessary to perform a QPCR reaction.

12. The kit of claim 11, wherein the kit comprises at least one thermostable polymerase.

13. A composition comprising a primer pair that is capable of amplifying a unique genomic sequence of interest, wherein the primers of the primer pair are engineered to specifically amplify the unique sequence of interest.

14. The composition of claim 13, wherein the primer pair amplifies a unique human genomic sequence.

15. A method of amplifying a unique genomic sequence of an organism in a method of amplifying a nucleic acid sequence of interest, said method comprising:
providing at least one pair of primers capable of amplifying a unique genomic sequence;
providing at least one probe or primer for amplification or detection of a nucleic acid sequence of interest; and
amplifying the unique genomic sequence and detecting the nucleic acid sequence of interest.

16. The method of claim 15, wherein the method comprises a PCR amplification method.

17. The method of claim 15, wherein the unique genomic sequence and the sequence of interest are amplified in the same reaction.

18. The method of claim 15, wherein any primer pair or pairs that amplify a unique genomic sequence can be used.

19. The method of claim 15, wherein the nucleic acid sequence of interest is an mRNA sequence.

20. The method of claim 15, wherein the nucleic acid sequence of interest is amplified using qRT-PCR.


SEQUENCE LISTING (50 sequences)

SEQ ID NO: 1 - DNA, 27 bp, Artificial Sequence
  Description of Artificial Sequence Synthetic primer
aacagaaatctggatgtgttattaagg

SEQ ID NO: 2 - DNA, 27 bp, Artificial Sequence
  Description of Artificial Sequence Synthetic primer
agaatagataagatgcagtcaccactt

SEQ ID NO: 3 - DNA, 27 bp, Artificial Sequence
  Description of Artificial Sequence Synthetic primer
gcctatttctcctggtagtttagaaat

SEQ ID NO: 4 - DNA, 27 bp, Artificial Sequence
  Description of Artificial Sequence Synthetic primer
ctgtacaacacagcttaaatgaggtta

SEQ ID NO: 5 - DNA, 27 bp, Artificial Sequence
  Description of Artificial Sequence Synthetic primer
aagtctatatctccaaacaagtcctca

SEQ ID NO: 6 - DNA, 27 bp, Artificial Sequence
  Description of Artificial Sequence Synthetic primer
gaataggatagcaatgatgttcaaagt

SEQ ID NO: 7 - DNA, 27 bp, Artificial Sequence
  Description of Artificial Sequence Synthetic primer
tcttgttcttgtcagttctctaaatca

SEQ ID NO: 8 - DNA, 27 bp, Artificial Sequence
  Description of Artificial Sequence Synthetic primer
ttgttatatacctgcattcaatcagaa

SEQ ID NO: 9 - DNA, 27 bp, Artificial Sequence
  Description of Artificial Sequence Synthetic primer
aactcctaactgataaaggttctggat

SEQ ID NO: 10 - DNA, 27 bp, Artificial Sequence
  Description of Artificial Sequence Synthetic primer
tgagaacacaaagagttgtttctaatg

SEQ ID NO: 11 - DNA, 27 bp, Artificial Sequence
  Description of Artificial Sequence Synthetic primer
aatgaatattcttcttacccacgtaga

SEQ ID NO: 12 - DNA, 27 bp, Artificial Sequence
  Description of Artificial Sequence Synthetic primer
ctgcaaatttaactatcaaatgacaaa

SEQ ID NO: 13 - DNA, 27 bp, Artificial Sequence
  Description of Artificial Sequence Synthetic primer
cttgaatttctcttctgtggtctaatc

SEQ ID NO: 14 - DNA, 27 bp, Artificial Sequence
  Description of Artificial Sequence Synthetic primer
tcccttaatataaagtacaaattgcgt

SEQ ID NO: 15 - DNA, 27 bp, Artificial Sequence
  Description of Artificial Sequence Synthetic primer
aaattctcctagcattcaaacctactt

SEQ ID NO: 16 - DNA, 27 bp, Artificial Sequence
  Description of Artificial Sequence Synthetic primer
gttgacctttcttatggttgcttatag

SEQ ID NO: 17 - DNA, 27 bp, Artificial Sequence
  Description of Artificial Sequence Synthetic primer
tgtgatctaaagggtgtgtgtataatc

SEQ ID NO: 18 - DNA, 27 bp, Artificial Sequence
  Description of Artificial Sequence Synthetic primer
cttagtatgagccatcaggagataaag

SEQ ID NO: 19 - DNA, 27 bp, Artificial Sequence
  Description of Artificial Sequence Synthetic primer
tataagaaactactaagcacccaaagg

SEQ ID NO: 20 - DNA, 27 bp, Artificial Sequence
  Description of Artificial Sequence Synthetic primer
aagaaaggagtctaagtgactcaacag

SEQ ID NO: 21 - DNA, 27 bp, Artificial Sequence
  Description of Artificial Sequence Synthetic primer
gtttgtacagactccgtaagatttgtt

SEQ ID NO: 22 - DNA, 34 bp, Artificial Sequence
  Description of Artificial Sequence Synthetic primer
aatgaccagtacaatttccctttatcactaaaaa

SEQ ID NO: 23 - DNA, 27 bp, Artificial Sequence
  Description of Artificial Sequence Synthetic primer
atatttctgagatttactgaatgacgg

SEQ ID NO: 24 - DNA, 34 bp, Artificial Sequence
  Description of Artificial Sequence Synthetic primer
acctggataagaccaccaactaatttcactttca

SEQ ID NO: 25 - DNA, 27 bp, Artificial Sequence
  Description of Artificial Sequence Synthetic primer
tgcttacagtgttaagtttagctgatg

SEQ ID NO: 26 - DNA, 34 bp, Artificial Sequence
  Description of Artificial Sequence Synthetic primer
ctatttagtcctaattcctactgagcattttccc

SEQ ID NO: 27 - DNA, 27 bp, Artificial Sequence
  Description of Artificial Sequence Synthetic primer
atatctaagagattcttgtgtgatgcc

SEQ ID NO: 28 - DNA, 32 bp, Artificial Sequence
  Description of Artificial Sequence Synthetic primer
aaggaaacccgttttctcagcctcaatctttc

SEQ ID NO: 29 - DNA, 27 bp, Artificial Sequence
  Description of Artificial Sequence Synthetic primer
atgtgccaaagtaatttagaattgaag

SEQ ID NO: 30 - DNA, 34 bp, Artificial Sequence
  Description of Artificial Sequence Synthetic primer
aacttatgaatgtcccaatagtgacccattttaa

SEQ ID NO: 31 - DNA, 27 bp, Artificial Sequence
  Description of Artificial Sequence Synthetic primer
actcatctatctaattacttcgccctt

SEQ ID NO: 32 - DNA, 34 bp, Artificial Sequence
  Description of Artificial Sequence Synthetic primer
tacaagcatagaaacaatacccatacactcctca

SEQ ID NO: 33 - DNA, 26 bp, Artificial Sequence
  Description of Artificial Sequence Synthetic primer
tttccaatgcagtcagataagaaata

SEQ ID NO: 34 - DNA, 34 bp, Artificial Sequence
  Description of Artificial Sequence Synthetic primer
acaaatataaaagcctgcattccttctattcatt

SEQ ID NO: 35 - DNA, 27 bp, Artificial Sequence
  Description of Artificial Sequence Synthetic primer
ggccatcaataaatatcaacttagaaa

SEQ ID NO: 36 - DNA, 34 bp, Artificial Sequence
  Description of Artificial Sequence Synthetic primer
cccagcactcttccaagcactgtataaatcatat

SEQ ID NO: 37 - DNA, 27 bp, Artificial Sequence
  Description of Artificial Sequence Synthetic primer
aatcttgaaaggtgaaggctaaatact

SEQ ID NO: 38 - DNA, 35 bp, Artificial Sequence
  Description of Artificial Sequence Synthetic primer
cagaaagctactagctgcatagtcttttcttacaa

SEQ ID NO: 39 - DNA, 27 bp, Artificial Sequence
  Description of Artificial Sequence Synthetic primer
tcaccattagctactcttacctatcct

SEQ ID NO: 40 - DNA, 34 bp, Artificial Sequence
  Description of Artificial Sequence Synthetic primer
aacatcaaatacccagtgtcctgcaaatgactgt

SEQ ID NO: 41 - DNA, 27 bp, Artificial Sequence
  Description of Artificial Sequence Synthetic primer
ggtgaagataatgaaagtcattggtat

SEQ ID NO: 42 - DNA, 27 bp, Artificial Sequence
  Description of Artificial Sequence Synthetic primer
taacagtaagagcatactggcagaaac

SEQ ID NO: 43 - DNA, 27 bp, Artificial Sequence
  Description of Artificial Sequence Synthetic primer
acacacatagtggtttatgaagaacct

SEQ ID NO: 44 - DNA, 27 bp, Artificial Sequence
  Description of Artificial Sequence Synthetic primer
taatatatagtctgttggcctcagtca

SEQ ID NO: 45 - DNA, 218 bp, Homo sapiens
ggtgaagataatgaaagtcattggtatttcttagatttttcatgctcaaaagtcacaagggactttgtaaactgaatctgattgatgataattgcaacctaaaagaagaggatttgaatttctgaagtttatgccagaactgacatctattctgattcctgttccaatcagtccttcattaaaagttgcctgtttctgccagtatgctcttactgtta

SEQ ID NO: 46 - DNA, 200 bp, Homo sapiens
acacacatagtggtttatgaagaacctgtcataaacctaaacgatagcaccaattagaaatgagcttccattgatatatttcagatcatttgcccttacccttttctgactttctgattctttaaactcatgaacatggttgagtgtatcatcgggtgatgaatagctagagatgactgaggccaacagactatatatta

SEQ ID NO: 47 - DNA, 261 bp, Homo sapiens
tgtgatctaaagggtgtgtgtataatccagaaagctactagctgcatagtcttttcttacaaagtatttagccttcacctttcaagatttccttcctctcaatatttggagaggacagaaggatagatattctaatactattttcatattggtctgtgcttttgagatcaccatgctcctttgaaaatactgccgggcgccctggcctctgtagaatagcagtgccagggaggcctttatctcctgatggctcatactaag

SEQ ID NO: 48 - DNA, 233 bp, Homo sapiens
tataagaaactactaagcacccaaaggaacatcaaatacccagtgtcctgcaaatgactgtaggataggtaagagtagctaatggtgatattaatgctgtataatacaatttaaaattagtatctctcctctttccatcacctaaattggcttactttccgtatttaaaattcacaataagtggcatcaacatgaatggagttggcctgttgagtcacttagactcctttctt

SEQ ID NO: 49 - DNA, 273 bp, Homo sapiens
aagtctatatctccaaacaagtcctcatagactatttagtcctaattcctactgagcattttccccatcagctaaacttaacactgtaagcatcattttcacctccatcctagatgctgctctctttctaagctgtcttacttctaccactatcattccttcagtctttaaggagagcatttatagtaatttttgactctttccttttccttaacctttaggcagttatcaaggcttactgtttttactttgaacatcattgctatcctattc

SEQ ID NO: 50 - DNA, 243 bp, Homo sapiens
gcctatttctcctggtagtttagaaatataattacctggataagaccaccaactaatttcactttcaccgtcattcagtaaatctcagaaatataagcaaagaacaatcttggacaagagaaaagaagaacctgatctcttttccagccctatgactcactgaagaaaccaggaatatgccacgtgttctctttctgctgcaagggttgctgtgaaataacctcatttaagctgtgttgtaca